(19) United States
(12) Patent Application Publication
(10) Pub. No.: US 2026/0308255 A1
(43) Pub. Date: Oct. 8, 2026

(54) MULTISPECIFIC ANTIBODIES TARGETING CD3 AND CD22

(71) Applicant: THE REGENTS OF THE UNIVERSITY OF CALIFORNIA, Oakland, CA (US)

(72) Inventors: Joseph M. TUSCANO, Oakland, CA (US); Joshua Farrell MECKLER, Oakland, CA (US); Gustavo BARISONE, Oakland, CA (US)

(21) Appl. No.: 19/485,253

(22) PCT Filed: May 17, 2024
(86) PCT No.: PCT/US2024/030121

§ 371 Date: Nov. 17, 2025

Related U.S. Application Data
(60) Provisional application No. 63/467,921, filed on May 19, 2023.

(51) Int. Cl. C07K 16/28; A61K 39/00; A61K 39/395; A61K 40/11; A61K 40/31; A61K 40/42; A61P 35/02

(52) U.S. Cl. CPC C07K 16/2809; A61K 39/3955; A61K 40/11; A61K 40/31; A61K 40/4211; A61P 35/02; C07K 16/2803; A61K 2039/505; C07K 2317/31; C07K 2317/565; C07K 2317/622; C07K 2317/73; C07K 2317/77

(57) ABSTRACT

Provided are CD22xCD3 and multispecific binding polypeptides that can include a cytokine domain. Pharmaceutical compositions and methods of treatment using the binding polypeptides to treat cancer are also provided.

### CROSS-REFERENCE TO RELATED APPLICATIONS

This application claims the benefit of U.S. Provisional Application No. 63/467,921, filed May 19, 2023, which is incorporated herein by reference in its entirety.

### SEQUENCE LISTING

The application contains a Sequence Listing which has been submitted electronically in .XML format and is hereby incorporated by reference in its entirety. Said .XML copy, created on May 16, 2024, is named “043882.00090.xml” and is 155,089 bytes in size. The sequence listing contained in this .XML file is part of the specification and is hereby incorporated by reference herein in its entirety.

### FIELD OF THE INVENTION

The present disclosure generally relates to multispecific, including bispecific, binding polypeptides that target CD3 and CD22 and, in some instances, can include a cytokine and compositions comprising the binding polypeptides, including pharmaceutical compositions, diagnostic compositions, and kits. The present disclosure also generally relates to multispecific binding polypeptides that target a first target and a second target and include a cytokine domain, in some instances, a sushi domain. Also provided are methods of making multispecific binding polypeptides, and methods of using multispecific binding polypeptides, for example, for therapeutic purposes, diagnostic purposes, and research purposes.

### BACKGROUND

Immunotherapy has revolutionized cancer therapy. Two recently FDA-approved immunotherapies for B-cell malignancies target CD19, in the form of a Bispecific T-Cell Engager (BiTE) antibody construct or chimeric antigen receptor T (CAR-T) cells. Blinatumomab, an FDA-approved BiTE, binds to CD19 on B cells and to CD3 on T cells, mediating effector-target cell contact and T-cell activation that results in effective elimination of target B cells. Although CD19 is expressed by essentially all B-cell malignancies at clinical presentation, relapses with loss or reduction of CD19 surface expression are increasingly recognized as a cause of treatment failure. Therefore, there is a clear need to develop therapeutics for alternate targets.

Breakthrough immunotherapy treatments such as rituximab, and more recently CAR-T therapies, have significantly extended survival for non-Hodgkin's lymphoma (NHL) and acute lymphocytic leukemia (ALL) patients. An additional treatment modality exists in bispecific antibodies (BisAbs), the concept of which dates to the 1960s when Alfred Nisonoff envisioned the potential of replacing one of the two identical antigen binding arms with a different antigen binding specificity [Fudenberg H H, Drews G, Nisonoff A. (1964) Serologic demonstration of dual specificity of rabbit bivalent hybrid antibody. J Exp Med. 119(1):151-166]. Many BisAbs have been developed with broad therapeutic potential due to their ability to target a variety of surface antigens that distinguish different cell or tissue types. More than 100 BisAbs have entered clinical trials as of early 2021 for conditions such as cancer and rheumatoid arthritis, utilizing at least 19 different protein architectures binding two or more surface antigens [Ma Jiabiang, et al. (2021) Bispecific Antibodies: From Research to Clinical Application Front. Immunol. 12]. Recently, bispecific antibodies have garnered favor for activating effector CD4/CD8 T cells through engagement of the CD3 antigen, ubiquitously expressed as a component of the T-cell receptor [Segal D M, Weiner G J, Weiner L M. (1999) Bispecific antibodies in cancer therapy. Curr. Opin. Immunol. 11:558-62]. Engagement with CD3 through BisAb crosslinking not only co-localizes the T cell to the target cell, but simultaneously activates the effector cell and stimulates proliferation [Liu L, Lam C K, Long V, Widjaja L, Yang Y, Li H, Jin L, Burke S, Gorlatov S, et. Al (2017) MGD011, A CD19x CD3 Dual-Affinity Retargeting Bi-specific Molecule Incorporating Extended Circulating Half-life for the Treatment of B-Cell Malignancies. Clin Cancer Res. 23(6):1506-1518; Choi B D, Cai M, Bigner D D, Mehta A I, Kuan C T, Sampson J H. (2011) Bispecific antibodies engage T cells for antitumor immunotherapy. Expert. Opin. Biol. Ther. 11:843-53]. This engagement facilitates a T-cell mediated immune response that corelates with tumor cell clearance through direct tumor cytotoxicity or activation of apoptotic cell pathways [Choi B D, Cai M, Bigner D D, Mehta A I, Kuan C T, Sampson J H. (2011) Bispecific antibodies engage T cells for antitumor immunotherapy. Expert. Opin. Biol. Ther. 11:843-53].

In many ways, the prototype bispecific T cell engager antibody is blinatumomab (blina), an αCD3xαCD19 BiTE targeting B cells expressing CD19. Blina is FDA approved for pediatric and adult ALL but has also demonstrated efficacy in other hematological malignancies [Goebeler M E, Knop S, Viardot A, Kufer P, Topp M S, Einsele H, Noppeney R, Hess G, Kallert S, et Al. (2016) Bispecific T-Cell Engager (BiTE) Antibody Construct Blinatumomab for the Treatment of Patients With Relapsed/Refractory Non-Hodgkin Lymphoma: Final Results From a Phase I Study. Journal of Clinical Oncology 34:10, 1104-1111; Zhu M, Wu B, Brandl C, Johnson J, Wolf A, Chow A, Doshi S. (2016) Blinatumomab, a Bispecific T-cell Engager (BiTE(®)) for CD-19 Targeted Cancer Immunotherapy: Clinical Pharmacology and Its Implications. Clin Pharmacokinet. 55(10):1271-1288]. Despite a high initial overall response rate, many patients relapse or become refractory to treatment [Jacoby E. (2019) Relapse and Resistance to CAR-T Cells and Blinatumomab in Hematologic Malignancies. Clin. Hematol. Int. 1(2):79-84]. Reduction in response may be due to low CD19 antigen expression or antigen loss from alternative splicing, non-functional membrane chaperone proteins, transformation to myeloid lineages, or CD19 mutations [Myers R M, Taraseviciute A, Steinberg S M, et al. (2022) Blinatumomab Nonresponse and High-Disease Burden Are Associated with Inferior Outcomes After CD19-CAR for B-ALL. J Clin Oncol. 40(9):932-944; Braig F, Brandt A, Goebeler M, Tony H-P, Kurze A-K, Nollau P, et al. (2017) Resistance to anti-CD19/CD3 BiTE in acute lymphoblastic leukemia may be mediated by disrupted CD19 membrane trafficking. Blood 129:100-4]. Similar mechanisms of resistance develop in response to CD19-targeted CAR-T therapeutics [Sotillo, E., et Al (2015) Convergence of Acquired Mutations and Alternative Splicing of CD19 Enables Resistance to CART-19 Immunotherapy. Cancer Discov. 5(12): p. 1282-95]. Because of these changes, CD19-directed therapies in the relapsed/refractory (R/R) setting may demonstrate limited efficacy with eventual relapse in most patients. Further, blinatumomab therapy presents neurotoxicity or cytokine release syndrome risk to some patients [Amgen, Data on file. www.blincytohcp.com/rr/safety/adult?gclid-CjwKCAjw2rmWBhB4EiwAiJ0mte8Vn4fxhC6 Wi7kVyQ8R-vsMZIx7hnAh-sem_410FC69bQxDP-KKjRoCcFOQAvD_BwE&gclsrc=aw.ds; accessed 12 Jul. 2022]. Several more recent alternative BiTEs target B-cell surface antigens such as CD20, CD38, and B-cell maturation antigen (BCMA). Some of these have entered clinical trials for treatment of B-cell lymphomas, multiple myeloma and leukemias [Ma Jiabiang, et al. (2021) Bispecific Antibodies: From Research to Clinical Application Front. Immunol. 12], with some reports of significant efficacy in early trials [Budde L E, Assouline S, Sehn L H, Schuster S J, Yoon S S, Yoon D H, Matasar M J, Bosch F, Kim W S, et Al. (2022) Single-Agent Mosunetuzumab Shows Durable Complete Responses in Patients With Relapsed or Refractory B-Cell Lymphomas: Phase I Dose-Escalation Study. J Clin Oncol. 40(5):481-491; Hutchings M, Morschhauser F, Iacoboni G, Carlo-Stella C, Offner F, Sureda A, Salles G, et Al. (2021) Glofitamab, a Novel, Bivalent CD20-Targeting T-Cell-Engaging Bispecific Antibody, Induces Durable Complete Remissions in Relapsed or Refractory B-Cell Lymphoma: A Phase I Trial. Journal of Clinical Oncology 39:18, 1959-1970].

Another attractive target in hematological malignancies is CD22, which is broadly expressed on B-cells [Shah, N N, et al. (2015) Characterization of CD22 expression in acute lymphoblastic leukemia. Pediatr Blood Cancer. 62(6): 964-9; Clark E A and Giltiay N V. (2018) CD22: A Regulator of Innate and Adaptive B Cell Responses and Autoimmunity. Front. Immunol. 9:2235; Clark E A. (1993) CD22, a B cell-specific receptor, mediates adhesion and signal transduction. J Immunol. 150:4715-8]. CD22 is a trans-membrane glycoprotein with internal tyrosine inhibitor motif (ITIM) domains and external sialic acid binding ligand. In B-cells, it functions generally in B-cell receptor (BCR) signaling to inhibit activation through recruitment of SHP-1 and other phosphatases to the cell membrane [Nitschke, L, et al., CD22 is a negative regulator of B-cell receptor signaling. Curr Biol, 1997. 7(2): p. 133-43]. Various CD22-targeted therapies using constructs such as monoclonal antibodies, antibody drug conjugates (ADCs), and more recently CD22 CAR-Ts have been developed. [Yoshida, S, et Al. (2017) Efficacy of an anti-CD22 antibody-monomethyl auristatin E conjugate in a preclinical xenograft model of precursor B-cell acute lymphoblastic leukemia. Leuk. Lymphoma. 58(5):1254-1257; Kantarjian H M, DeAngelo D J, Stelljes M, et Al. (2019) Inotuzumab ozogamicin versus standard of care in relapsed or refractory acute lymphoblastic leukemia: final report and long-term survival follow-up from the randomized, phase 3 INO-VATE study. Cancer. 125(14):2474-2487; O'Donnell, R T, et Al. (2009) Treatment of non-Hodgkin's lymphoma xenografts with the HB22.7 anti-CD22 monoclonal antibody and phosphatase inhibitors improves efficacy. Cancer Immunol Immunother. 58(10):1715-22; O'Donnell, R T, et Al. (2009) Phosphatase inhibition augments anti-CD22-mediated signaling and cytotoxicity in non-hodgkin's lymphoma cells. Leuk Res. 33(7):964-9; Spiegel J Y, Patel S, Muffly, L et Al. (2021) CAR T cells with dual targeting of CD19 and CD22 in adult patients with recurrent or refractory B cell malignancies: a phase 1 trial. Nat. Med. 27:1419-1431; Martin, S M, et Al. (2011) The HB22.7 Anti-CD22 monoclonal antibody enhances bortezomib-mediated lymphomacidal activity in a sequence dependent manner. J Hematol Oncol. 4:49].

CD22-directed treatments have potential to serve as alternatives or complements to CD19- and/or CD20-targeted therapies. Additionally, they can be a treatment option for patients who no longer respond to drugs targeting those antigens. Indeed, in one trial, patients relapsed or refractory to blinatumomab who were then treated with the CD22 ADC inotuzumab experienced 68% complete response as a bridge therapy to hemopoietic stem cell treatment (HSCT) [Wudhikarn K, King A C, Geyer M B, Roshal M, Bernal Y, Gyurkocza B, Perales M-A, Park J H. (2022) Outcomes of relapsed B-cell acute lymphoblastic leukemia after sequential treatment with blinatumomab and inotuzumab. Blood Advances. 6(5): 1432-1443]. Additionally, CD22-targeted CAR-T therapies have demonstrated sustained complete responses in patients previously treated with blina or CD19 CAR-T [Zhang, Y, Li, S, Wang, Y et Al. (2022) A novel and efficient CD22 CAR-T therapy induced a robust antitumor effect in relapsed/refractory leukemia patients when combined with CD19 CAR-T treatment as a sequential therapy. Exp. Hematol. Oncol. 11, 15].

The sushi domain-IL-15 complex is known to be a “superagonist” of NK and CD8⁺ T cells, enhancing their proliferation and anti-tumor activity and can be delivered as untargeted reagents, or as presented here, fused to antibody constructs to enable targeted delivery of cytokines which can further benefit cancer therapy. Thus, there is a need for compositions that can provide enhanced proliferation and anti-tumor activity.

### SUMMARY

Provided herein are multispecific binding polypeptides, e.g. antibodies, that selectively bind human CD3 and human CD22. In some embodiments, the multispecific binding polypeptides further comprise a cytokine domain.

In one aspect, the present disclosure provides a novel αCD22xαCD3 BiTE binding polypeptide that has significant in vitro and in vivo activity against ALL and may synergize with blinatumomab. The construct performed similarly to blinatumomab in in vitro cytotoxicity studies using ALL/NHL cell lines. Additionally, the αCD22xαCD3 BiTE, at equivalent molar dosages to blinatumomab, demonstrated activation of effector T-cells at levels comparable to blinatumomab (blina). In vivo data suggests that the construct prolongs survival of mice with ALL xenografts, with minimal toxicity, and demonstrates performance equal to, or exceeding, blinatumomab. It was also surprisingly found that a combination of αCD22xαCD3 BiTE and blinatumomab exhibits an enhanced anti-tumor effect in our mouse model, warranting further investigation of dually targeting CD19/CD22 through T-cell engaging treatment modalities. Thus, the novel αCD22xαCD3 BiTE may offer an alternative or companion therapy to CD19-based treatments that can be used in lymphoma therapeutic courses.

The αCD22xαCD3 BiTE promotes in vitro cell-mediated cytotoxicity in a dose and effector:target (E:T)-dependent fashion. Additionally, in an established acute lymphoblastic leukemia (ALL) xenograft mouse model, the CD22-BiTE demonstrates tumor growth inhibition, comparable to blinatumomab. Further, the combination of blinatumomab and CD22-BiTE yields increased efficacy in vivo when compared to the single agents. The BiTE represents an alternate or complementary therapeutic option for B-cell malignancies.

In some aspects, a CD22xCD3 binding polypeptide comprising a CD22 binding domain and a CD3 binding domain is provided wherein the CD22 binding domain comprises a first variable heavy chain comprising heavy chain complementarity determining region (HCDR) 1, HCDR2 and HCDR3 of SEQ ID NO: 1 and a first variable light chain comprising light chain complementarity determining region (LCDR) 1, LCDR2 and LCDR3 of SEQ ID NO: 8, and wherein the CD3 binding domain comprises a second variable heavy chain comprising heavy chain complementarity determining region (HCDR) 1, HCDR2 and HCDR3 of SEQ ID NO: 16 and a second variable light chain comprising light chain complementarity determining region (LCDR) 1, LCDR2 and LCDR3 of SEQ ID NO: 23.

In some aspects, a CD22xCD3 binding polypeptide comprising a CD22 binding domain and a CD3 binding domain is provided wherein the CD22 binding domain comprises a first variable heavy chain comprising a sequence having at least 80% sequence identity to SEQ ID NO: 1 and a first variable light chain comprising a sequence having at least 80% sequence identity to SEQ ID NO: 8, and wherein the CD3 binding domain comprises a second variable heavy chain comprising a sequence having at least 80% sequence identity to SEQ ID NO: 16 and a second variable light chain comprising a sequence having at least 80% sequence identity to SEQ ID NO: 23.

In some aspects, a CD22xCD3 binding polypeptide comprising a CD22 binding domain and a CD3 binding domain, wherein the CD22 binding domain is capable of blocking binding of the natural ligand to CD22.

In some aspects, a CD22xCD3 binding polypeptide comprising the sequence of SEQ ID NO: 31 is provided.

In some aspects, a CD22xCD3 binding polypeptide comprising the sequence of SEQ ID NO: 52 is provided.

In some aspects, a CD22xCD3 binding polypeptide comprising the sequence of SEQ ID NO: 91 is provided.

In some aspects, a CD22xCD3 binding polypeptide comprising the sequence of SEQ ID NO: 96 is provided.

In some aspects, a multispecific binding polypeptide comprising a first binding domain that specifically binds to CD3, a second binding domain that specifically binds to CD22, and a cytokine domain comprising a cytokine or a mutant, variant, or functional fragment thereof is provided.

In some aspects, a method for treating cancer in a subject in need thereof is provided, comprising: administering a therapeutically effective amount of a CD22xCD3 binding polypeptide or multispecific binding polypeptide of the disclosure to the subject.

In some aspects, a method for producing a CD22xCD3 binding polypeptide or multispecific binding polypeptide of the present disclosure is provided that comprises incubating a host cell comprising a polynucleotide or expression vector encoding the CD22xCD3 binding polypeptide or multispecific binding polypeptide under conditions sufficient to yield the CD22xCD3 binding polypeptide or multispecific binding polypeptide.

It is an object of the present disclosure that the multispecific binding polypeptides, optionally including the cytokine domain, would serve to co-localize effector T-cells (CD3⁺), target tumor B-cells (CD22⁺), and would simultaneously enhance the tumor killing and proliferation of the T-cells. This would be predicated to improve the anti-tumor response compared to a bispecific antibody without the cytokine domain.

It is a second object of the present disclosure that the multispecific binding polypeptides, including the cytokine domain, would reduce the risk of toxicities that can occur with systemic cytokine, e.g. IL-15, delivery by including a combination of at least three moieties in a single molecule.

It is a third object of the present disclosure that the cytokine domain, e.g. IL-15/Sushi component can also enhance NK cell activity and, therefore, may enhance the local immune response beyond just T cells. In other words, NK cells that are local to the tumor may also be activated by the IL-15 superagonist. Some data suggests that CD56⁺ (NK) cells are activated in vitro by this molecule.

It is a fourth object of the present disclosure that T-cells activated with the cytokine domain, e.g. IL-15 superagonist have enhanced activity, proliferation and better retention of memory T-cells.

It is a fifth object of the present disclosure that the CD22 binding domain can have “blocking” activity, meaning that it blocks CD22 from interacting with its ligand which has been shown to have immunostimulatory activity and, for multispecific binding polypeptides including the cytokine domain may display synergistic activity.

It is a sixth object of the present disclosure that different cytokines can be used in the cytokine domain, e.g. IL-12 could be introduced, such as between the CD22 binding domain and the CD3 binding domain, or other cytokines such as IL-2 or an IL-2 “superkine” could be used as the cytokine.

It is a seventh object of the present disclosure that different binding domains could be used against other targets in the multispecific binding polypeptide including the cytokine domain, e.g. the IL-15 sushi domain.

It is an eighth object of the present disclosure that different configurations of a multispecific binding polypeptide can be formed such as including the cytokine domain at a carboxy-terminal end instead of between the two binding domains.

It is a ninth object of the present disclosure that the multispecific binding polypeptides that include the cytokine domain can have superior anti-tumor effect over bispecific molecules that do not include the cytokine binding domain, such as blinatumomab.

It is a tenth object of the present disclosure that the binding polypeptides and multispecific antibodies of the present disclosure can be administered to a subject in combination with CAR-T cells to enhance the effectiveness of the CAR-T therapy.

These and other embodiments of the disclosure along with many of its features are described in more detail in conjunction with the text below and attached figures.

### BRIEF DESCRIPTION OF THE FIGURES

FIG. 1 provides a comparison of the Kabat and Chothia numbering systems for CDR-H1. Adapted from Martin A. C. R. (2010). Protein Sequence and Structure Analysis of Antibody Variable Domains. In R. Kontermann & S. Dübel (Eds.), Antibody Engineering vol. 2 (pp. 33-51). Springer-Verlag, Berlin Heidelberg.

FIG. 2 provides a graphical representation of the BiTE constructs. ScFv fragments for human αCD22 antigen and αCD3 antigen were placed in sequence to produce a BiTE molecule with similar architecture to blinatumomab.

FIG. 3 provides a SDS-PAGE gel with Coomassie blue staining of the crude and fractions from Ni-NTA purification of BiTE-197. Samples were run under reduced, denaturing conditions. Lanes are labeled as follow: Cr=crude culture supernatant; FT=Flow through (unbound) fraction; W=wash fractions; E=elution fractions. Relative molecular weights (in kDa) are indicated on the left. Predicted molecular weight=53.9 kDa.

FIG. 4 provides flow cytometry data for the binding of αCD22xαCD3 BiTE constructs to CD3 or CD22 targets. To test binding of BiTE 197 to its CD22 cognate target, cells were incubated with BiTE 197 at 1 ug/mL and then probed with a secondary anti-His-PE before flow cytometry analysis. K562 cells do not natively express CD22 as confirmed with a research grade αCD22-BV421 antibody (B) and BiTE 197 (A). K562 cells were tranduced to express CD22 (D) and binding of BiTE 197 was confirmed (C). Raji cells natively express surface CD22 in high quantity and BiTE 197 demonstrated binding to Raji cells (E) as did a control αCD22-BV421 antibody (F). Finally, T cells isolated from healthy PBMCs were incubated with either BiTE 197 then αHis-PE (G) or αCD3-AlexaFluor 488 (H) to confirm binding to CD3 target.

FIG. 5 provides cell killing data for Granta NHL cells.

FIG. 6 provides cells killing data for HT1080 sarcoma cells, a non B cell which has been shown to express CD22.

FIG. 7 provides T-cell activation data. T-cell 24 h activation with BiTE #197. 50,000 T cells were plated with CFSE labeled target NALM-6 cells at E:T 1:1 and treated with either blinatumomab (0.1 ug/mL), BiTE 197 (0.1 ug/mL), Blinatumomab+197, or target cells alone. Cells were plated without IL-2. Cells were incubated for 24 hours then analyzed by Flow Cytometry identifying CFSE-single cells A) total T cells, B) CD4, or C) CD8-gated T cells positive for CD69 and CD25. Median Fluorescence Intensity (MFI) in the T cell population positive for either CD69 or CD25 was used to quantify activation. Significant difference between groups was determined using 1-way Anova with Tukey's multiple comparison test. *p<0.05; **p<0.001; ***p<0.0001; ****p<0.00001.

FIG. 8 provides specific killing of tumor cells in vitro. 50,000 CFSE-labeled target cells were incubated with PBMC effector cells at the indicated E:T ratios and antibody concentrations using A) NALM-6 and B) U2973 cells as targets. After 24 hours, target cell lysis was measured by flow cytometry. Killing was determined by proportion of cells positive for CSFE/ZNIR out of all CSFE positive cells. All treatment groups were significantly different from the untreated control by unpaired t-test *p<. 05; **p<0.001; ***p<0.0001.

FIG. 9 provides specific killing of tumor cells in vitro (A). 50,000 NALM-6 Cells were incubated with PBMC effector cells at the indicated E:T ratios and antibody concentrations in the presence of 500 U/mL of IL-2. B) the assay was repeated with U2973 cells. (C and D) Additional trial with separate PBMC donors using the same conditions as FIGS. 8A-8B. Briefly, 50,000 C) NALM-6 or D) U2973 target cells. No IL-2 was added for the experiments in C and D.

FIG. 10 provides the study design for an in vivo mouse study using BiTE #197.

FIG. 11 provides A) Study 1: NSG mice were inoculated with 10⁴ NALM-6 cells and 10⁷ PBMCs through S.C. injection and treated with BiTE construct 197, blinatumomab, or PBS (negative control) daily for 5 days. N=6 for each group. Mice were euthanized upon significant weight loss or hind limb paralysis. B) Study 2: NSG mice were inoculated with 10⁴ NALM-6 cells and 10⁷ PBMCs IV through tail vein injection and treated with BiTE construct 197, blinatumomab, 197+blinatumomab or PBS (negative control) daily for 5 days. N=6 for each group. Mice were euthanized upon significant weight loss or hind limb paralysis. Survival was significantly different between treated and untreated groups by log rank Mantel-Cox significance test.

FIG. 12 provides physiology data for an in vivo mouse study.

FIG. 13 provides CBC data for an in vivo mouse study.

FIG. 14 provides an SDS-PAGE gel with Coomassie blue staining of clinical grade Blinantumomab (blina). purified BiTE-197, purified tri-functional antibody (also referred to as construct ‘301’), along with dilution standards of Bovine Serum Albumin (BSA) (lanes 5-10). Samples were run under reduced, denaturing conditions. Predicted molecular weight for blina and 197=53.9 kDa. Predicted molecular weight for the 301 tri-functional engager=79 kDa. Also included is the quantification of the antibody concentration based on band densities from the gel.

FIG. 15 provides survival data from the same study as shown in FIG. 11B, including the treatment arm in which mice were given the tri-functional engager 301 at 1 ug/mouse.

FIG. 16 depicts the results of in vivo mouse study in which Blina, BiTE 197 and tri-functional engager 301 were compared. This study was performed in the exact same manner as the study depicted in FIG. 15 and confirms the superiority of both the tri-functional engager alone, and the combined Blina+197 treatment, compared to treatment with just the single FDA-approved agent Blina, or to 197 alone.

FIGS. 17A-17F present a panel of overnight cytotoxicity assays against a variety of malignant b-cell lines (Nalm6, SU-DHL10, U2973). The flow-based killing assays were performed as described in example 5. Deviations in the effector:target (ET) ratios, amount of cells plated, presence or absence of exogenous IL-2, and amounts of treatments (i.e. antibodies, and exogenous IL-15) that were added, are noted in the various figures.

FIGS. 18A-18C illustrate that BITE 197 and the tri-functional engager 301 can upregulate activation markers CD25, CD69 and Tumor Necrosis Factor Alpha (TNFa) to varying degrees in a 24-hour co-culture of peripheral blood mononuclear cells (PBMCs) and target NALM6 or U2973 cells, supplemented with the indicated treatments.

FIGS. 19A-19C present data from another in vitro activation experiment, where activation markers CD25 and CD69 were investigated in various subsets of the PBMC effector cells

FIGS. 20A-20G showcase data from another set of in vitro overnight activation experiments. In this set, the target cells were labeled with CFSE, and the effector cells consisted of purified T cells, rather than a mixture of PBMCs.

FIG. 21 shows the results of an antibody titration for tri-functional engager 301 in an overnight cytotoxicity assay (performed as in example 5).

FIG. 22A illustrates cytotoxic effects of BiTE-197, tri-functional engager 301 and an αCD22-IL-12-αCD3 tri-functional engager.

FIGS. 22B-22G illustrate T-cell activation by BiTE-197, tri-functional engager 301 and an αCD22-IL-12-αCD3 tri-functional engager.

FIG. 23A illustrates cytotoxic effects of BiTE-197, tri-functional engager 301 and an αCD22-IL-12-αCD3 tri-functional engager co-incubated with anti-CD19 CAR-T cells.

FIGS. 23B-23C illustrate flow cytometry data for CD69 and CD25, respectively, for the four treatments in Example 9.

FIG. 24 illustrates Green Integrated Intensity per Image over time for Example 10.

FIG. 25 illustrates Green Integrated Intensity per Image over time for Example 11.

FIG. 26A illustrates cytotoxic effects of BiTE-197, tri-functional engager 301 and an αCD22-IL-12-αCD3 tri-functional engager against Nalm6-GFP-Luc cells when co-incubated with PBMCs.

FIG. 26B illustrates cytotoxic effects of BiTE-197, tri-functional engager 301 and an αCD22-IL-12-αCD3 tri-functional engager against Raji cells when co-incubated with PBMCs.

### DETAILED DESCRIPTION OF THE EMBODIMENTS

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2^{nd} ed. (1989) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, ±5%, or ±1%. In certain embodiments, the term “about” indicates the designated value±one standard deviation of that value.

The term “combinations thereof” includes every possible combination of elements to which the term refers to. For example, a sentence stating that “if α2 is A, then α3 is not D; α5 is not S; or α6 is not S; or combinations thereof” includes the following combinations when α2 is A: (1) α3 is not D; (2) α5 is not S; (3) α6 is not S; (4) α3 is not D; α5 is not S; and α6 is not S; (5) α3 is not D and α5 is not S; (6) α3 is not D and α6 is not S; and (7) α5 is not S and α6 is not S.

As used herein, “CD3” refers to T-cell receptor CD3. In some embodiments, CD3 refers to the epsilon chain of CD3. In some embodiments CD3 refers to a protein comprising the sequence of SEQ ID NO: 56 or a portion thereof, such as the extracellular portion. It should be understood that CD3 can more broadly refer to other CD3 chains.

As used herein, “CD19” refers to B-lymphocyte antigen CD19 which can also be known as B-Lymphocyte Surface Antigen B4, T-cell Surface Antigen Leu-12 or CVID3.

As used herein, “CD20” refers to B-lymphocyte antigen CD20.

As used herein, “CD22” refers to B-cell receptor CD22. In some embodiments, CD22 refers to a protein comprising the sequence of SEQ ID NO: 55 or a portion thereof, such as the extracellular portion.

As used herein, “BiTE 197,” “BITE #197,” “197” and the like refer to a bispecific antibody having the sequence of SEQ ID NO: 57 minus the signal peptide.

As used herein, “BiTE 198,” “BITE #198,” “198” and the like refer to a bispecific antibody having the sequence of SEQ ID NO: 79 minus the signal peptide (SEQ ID NO: 59).

As used herein “tri-functional engager 301,” “301,” and the like refer to a trifunctional binding polypeptide having the sequence of SEQ ID NO: 67 minus the signal peptide and linker 6xHis-tag (SEQ ID NO: 52). As used herein, tri-functional engager 301 is also referred to as “IL15 Trike” or the like.

As used herein “IL-12 Trike” and the like refer to a trifunctional binding polypeptide having the sequence of SEQ ID NO: 103 minus the signal peptide and linker 6xHis-tag (SEQ ID NO: 96).

As used herein, the term “chimeric antigen receptor (CAR)” refers to artificial T-cell receptors, T-bodies, single-chain immunoreceptors, chimeric T-cell receptors, or chimeric immunoreceptors, for example, and encompass engineered receptors that graft an artificial specificity onto a particular immune cell.

As used herein, the term “chimeric antigen receptor (CAR)-T cell” refers to a T cell that has been engineered to express a chimeric antigen receptor.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7^{th} ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5^{th} ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“Aho” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

TABLE 1
Residues in CDRs according to Kabat and Chothia
numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat | H31-H35B | H26-H32 or H34* |
| Numbering) | | |
| H1 (Chothia | H31-H35 | H26-H32 |
| Numbering) | | |
| H2 | H50-H65 | H52-H56 |
| H3 | H95-H102 | H95-H102 |

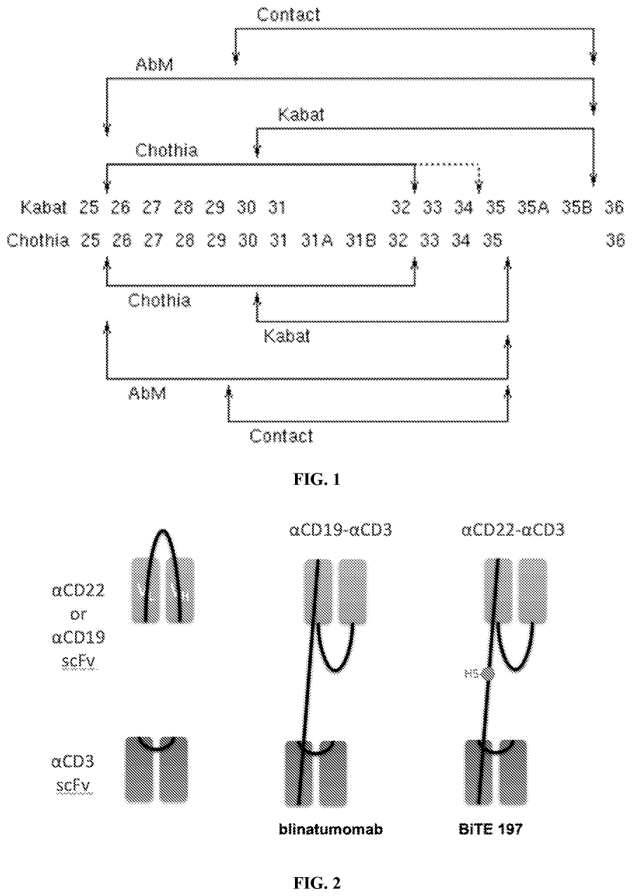

*The C-terminus of CDR-H1, when numbered using the Kabat numbering convention, varies between H32 and H34, depending on the length of the CDR, as illustrated in FIG. 1.

Unless otherwise specified, the numbering scheme used for identification of a particular CDR herein is the Kabat/Chothia numbering scheme. Where the residues encompassed by these two numbering schemes diverge (e.g., CDR-H1 and/or CDR-H2), the numbering scheme is specified as either Kabat or Chothia. For convenience, CDR-H3 is sometimes referred to herein as either Kabat or Chothia. However, this is not intended to imply differences in sequence where they do not exist, and one of skill in the art can readily confirm whether the sequences are the same or different by examining the sequences.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with ß-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). In some embodiments, the linker is SEQ ID NO: 38. Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminus of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG1 Fc domain.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule.

An “affinity matured” antibody is one with one or more alterations in one or more CDRs or FRs that result in an improvement in the affinity of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. Polypeptide sequences having such substitutions are known as “conservatively modified variants.” By way of example, the groups of amino acids provided in Tables 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Additional selected groups of amino
acids that are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that
are considered conservative substitutions for
one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2^{nd} ed. (1993) W. H. Freeman & Co., New York, NY. An antibody generated by making one or more conservative substitutions of amino acid residues in a parent antibody is referred to as a “conservatively modified variant.”

As used herein, the term “functional variant” or “functional fragment” refers to a molecule, including a binding molecule, for example, comprises a nucleotide and/or amino acid sequence that is altered by one or more nucleotides and/or amino acids compared to the nucleotide and/or amino acid sequences of the parent molecule. For a binding molecule, a functional variant is still capable of competing for binding to the binding partner with the parent binding molecule. In other words, the modifications in the amino acid and/or nucleotide sequence of the parent binding molecule do not significantly affect or alter the binding characteristics of the binding molecule encoded by the nucleotide sequence or containing the amino acid sequence, i.e., the binding molecule is still able to recognize and bind its target. The functional variant may have conservative sequence modifications including nucleotide and amino acid substitutions, additions and deletions. These modifications can be introduced by standard techniques known in the art, such as site-directed mutagenesis and random PCR-mediated mutagenesis.

Functional variants and fragments can also include, but are not limited to, derivatives that are substantially similar in primary structural sequence, but which contain e.g., in vitro or in vivo modifications, chemical and/or biochemical, that are not found in the parent binding molecule such modifications include inter alia acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI-anchor formation, hydroxylation, iodination, methylation, myristoylation, oxidation, pegylation, proteolytic processing, phosphorylation, prenylation, racemization, selenoylation, sulfation, transfer-RNA-mediated addition of amino acids to proteins such as arginylation, ubiquitination, and the like.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of an antibody or composition that when administered to a subject is effective to treat a disease or disorder.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human.

### 2, CD22xCD3 Binding Polypeptides

In some embodiments, a CD22xCD3 binding polypeptide comprises, consists of, or consists essentially of a CD22 binding domain comprising a first variable heavy chain and a first variable light chain and a CD3 binding domain comprising a second variable heavy chain and a second variable light chain.

### 2.1 CD22 Binding Domain

In some embodiments, the CD22 binding domain can bind human CD22 or a homolog thereof. In some embodiments, the CD22 binding domain can have blocking activity against the natural ligand for CD22.

### 2.1.1 HCDR Sequences

In some embodiments, the first variable heavy chain comprises a heavy chain complementarity determining region (HCDR) 1, HCDR2 and HCDR3 of SEQ ID NO: 1. In certain aspects, the HCDR1, HCDR2 and HCDR3 can be according to the Kabat or Chothia numbering scheme.

In some embodiments, the first variable heavy chain comprises a Kabat heavy chain complementarity determining region (HCDR) 3 comprising, consisting of, or consisting essentially of the sequence of SEQ ID NO: 2. In some embodiments, the first variable heavy chain comprises a Chothia HCDR3 comprising, consisting of, or consisting essentially of SEQ ID NO: 5.

In some embodiments, the first variable heavy chain comprises a Kabat HCDR2 comprising, consisting of, or consisting essentially of the sequence of SEQ ID NO: 3. In some embodiments, the first variable heavy chain comprises a Chothia HCDR2 comprising, consisting of, or consisting essentially of SEQ ID NO: 6.

In some embodiments, the first variable heavy chain comprises a Kabat HCDR1 comprising, consisting of, or consisting essentially of the sequence of SEQ ID NO: 4. In some embodiments, the first variable heavy chain comprises a Chothia HCDR1 comprising, consisting of, or consisting essentially of SEQ ID NO: 7.

### 2.1.2 LCDR Sequences

In some embodiments, the first variable light chain comprises a light chain complementarity determining region (LCDR) 1, LCDR2 and LCDR3 of SEQ ID NO: 8 according to the Kabat or Chothia numbering scheme.

In some embodiments, the first variable light chain comprises a Kabat light chain complementarity determining region (LCDR) 3 comprising, consisting of, or consisting essentially of the sequence of SEQ ID NO: 9. In some embodiments, the first variable light chain comprises a Chothia LCDR3 comprising, consisting of, or consisting essentially of SEQ ID NO: 12.

In some embodiments, the first variable light chain comprises a Kabat LCDR2 comprising, consisting of, or consisting essentially of the sequence of SEQ ID NO: 10. In some embodiments, the first variable light chain comprises a Chothia LCDR2 comprising, consisting of, or consisting essentially of SEQ ID NO: 13.

In some embodiments, the first variable light chain comprises a Kabat LCDR1 comprising, consisting of, or consisting essentially of the sequence of SEQ ID NO: 11. In some embodiments, the first variable light chain comprises a Chothia LCDR1 comprising, consisting of, or consisting essentially of SEQ ID NO: 14.

### 2.1.3 V_{H} Sequences

In some embodiments, the first variable heavy chain comprises, consists of, or consists essentially of a sequence having at least 70% sequence identity to SEQ ID NO: 1. In some embodiments, the first variable heavy chain comprises, consists of, or consists essentially of a sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the first variable heavy chain comprises, consists of, or consists essentially of the sequence of SEQ ID NO: 1.

### 2.1.4 V_{L} Sequences

In some embodiments, the first variable light chain comprises, consists of, or consists essentially of a sequence having at least 70% sequence identity to SEQ ID NO: 16. In some embodiments, the first variable light chain comprises, consists of, or consists essentially of a sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the first variable light chain comprises, consists of, or consists essentially of the sequence of SEQ ID NO: 16.

2.1.5 V_{H}/V_{L} Pair

In some embodiments, the first variable heavy chain comprises, consists of, or consists essentially of the sequence of SEQ ID NO: 1 and the first variable light chain comprises, consists of, or consists essentially of the sequence of SEQ ID NO: 8.

2.1.6 scFv Sequence

In some embodiments, the CD22 binding domain comprises, consists of, or consists essentially of a sequence having at least 70% sequence identity to SEQ ID NO: 15. In some embodiments, the CD22 binding domain comprises, consists of, or consists essentially of a sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 15. In some embodiments, the CD22 binding domain comprises, consists of, or consists essentially of the sequence of SEQ ID NO: 15.

### 2.1.7 Linkers

In some embodiments, the CD22 binding domain comprises a first linker positioned between the first variable heavy chain and the first variable light chain.

In some embodiments, the first linker comprises, consists of, or consists essentially of a flexible linker. In some embodiments, the first linker comprises, consists of, or consists essentially of a glycine/serine-rich linker. In some embodiments, the glycine/serine-rich linker comprises (GGGGS)ₙ, where n is 1, 2, 3, 4, 5, 6, 7 or 8. In some embodiments, the first linker comprises, consists of, or consists essentially of the sequence of any one of SEQ ID NO: 38, SEQ ID NO: 42, SEQ ID NO: 43, SEQ ID NO: 45, SEQ ID NO: 46, SEQ ID NO: 47, SEQ ID NO: 50, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99. In some embodiments, the first linker comprises, consists of, or consists essentially of the sequence of SEQ ID NO: 38. Exemplary linkers are disclosed in Chen, et al., “Fusion Protein Linkers: Property, Design and Functionality,” Adv Drug Deliv Rev. 2013 Oct. 15 65(10):1357-1369, Brinkmann U. and Kontermann, Roland, “The making of bispecific antibodies,” MAbs 2017 9(2):182-212, U.S. Patent Application Publication No. 2019/0092818 and International Patent Application Publication No. WO 2017/106462.

2.1.8 Arrangement of V_{H} and V_{L}

In some embodiments, the first variable heavy chain and the first variable light chain are oriented in order from N- to C-terminus of the CD22 binding domain. In some embodiments, the first variable light chain and the first variable heavy chain are oriented in order from N- to C-terminus.

### 2.1.9 Format of CD22 Binding Domain

In some embodiments, the CD22 binding domain is an antibody. In some embodiments, the CD22 binding domain is a Fv, scFv, Fab, F(ab′)2, or scFv-Fc. In some embodiments, the CD22 binding domain is a scFv. In some embodiments, the CD22 binding domain is a humanized antibody or fragment thereof. In some embodiments, the CD22 binding domain is a chimeric antibody or fragment thereof.

In some embodiments, the first variable heavy chain is an IgA. In some embodiments, the First variable heavy chain is an IgD. In some embodiments, the first variable heavy chain is an IgE. In some embodiments, the first variable heavy chain is an IgG. In some embodiments, the first variable heavy chain is an IgM. In some aspects, the first variable heavy chain is an IgG1. In some aspects, the first variable heavy chain is an IgG2. In some aspects, the first variable heavy chain is an IgG3. In some aspects, the first variable heavy chain is an IgG4. In some aspects, the first variable heavy chain is an IgA1. In some aspects, the first variable heavy chain is an IgA2.

In some embodiments, the first variable light chain is a kappa light chain. In some embodiments, the first variable light chain is a lambda light chain.

In some embodiments, the CD22 binding domain is a humanized antibody. In some embodiments, the CD22 binding domain is a chimeric antibody.

In some embodiments, the CD22 binding domain is an affinity matured antibody.

### 2.1.10 CDR Variants

In some embodiments, the CDRs of the CD22 binding domain have at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to those recited herein. In some embodiments, the CDRs of the CD22 binding domain differ by 1, 2 or 3 amino acid substitutions from the CDRs recited herein. In certain aspects, the substitutions are conservative substitutions.

### 2.2 CD3 Binding Domain

In some embodiments, the CD22 binding domain can bind human CD22 or a homolog thereof.

### 2.2.1 HCDR Sequences

In some embodiments, the second variable heavy chain comprises a heavy chain complementarity determining region (HCDR) 1, HCDR2 and HCDR3 of SEQ ID NO: 16. In certain aspects, the HCDR1, HCDR2 and HCDR3 can be according to the Kabat or Chothia numbering scheme.

In some embodiments, the second variable heavy chain comprises a Kabat heavy chain complementarity determining region (HCDR) 3 comprising, consisting of, or consisting essentially of the sequence of SEQ ID NO: 17. In some embodiments, the second variable heavy chain comprises a Chothia HCDR3 comprising, consisting of, or consisting essentially of SEQ ID NO: 20

In some embodiments, the second variable heavy chain comprises a Kabat HCDR2 comprising, consisting of, or consisting essentially of the sequence of SEQ ID NO: 18. In some embodiments, the second variable heavy chain comprises a Chothia HCDR2 comprising, consisting of, or consisting essentially of SEQ ID NO: 21.

In some embodiments, the second variable heavy chain comprises a Kabat HCDR1 comprising, consisting of, or consisting essentially of the sequence of SEQ ID NO: 19. In some embodiments, the second variable heavy chain comprises a Chothia HCDR1 comprising, consisting of, or consisting essentially of SEQ ID NO: 22.

### 2.2.2 LCDR Sequences

In some embodiments, the second variable light chain comprises a light chain complementarity determining region (LCDR) 1, LCDR2 and LCDR3 of SEQ ID NO: 23 according to the Kabat or Chothia numbering scheme.

In some embodiments, the second variable light chain comprises a Kabat light chain complementarity determining region (LCDR) 3 comprising, consisting of, or consisting essentially of the sequence of SEQ ID NO: 24. In some embodiments, the second variable light chain comprises a Chothia LCDR3 comprising, consisting of, or consisting essentially of SEQ ID NO: 27.

In some embodiments, the second variable light chain comprises a Kabat LCDR2 comprising, consisting of, or consisting essentially of the sequence of SEQ ID NO: 25. In some embodiments, the second variable light chain comprises a Chothia LCDR2 comprising, consisting of, or consisting essentially of SEQ ID NO: 28.

In some embodiments, the second variable light chain comprises a Kabat LCDR1 comprising, consisting of, or consisting essentially of the sequence of SEQ ID NO: 26. In some embodiments, the second variable light chain comprises a Chothia LCDR1 comprising, consisting of, or consisting essentially of SEQ ID NO: 29.

### 2.2.3 V_{H} Sequences

In some embodiments, the second variable heavy chain comprises, consists of, or consists essentially of a sequence having at least 70% sequence identity to SEQ ID NO: 1. In some embodiments, the second variable heavy chain comprises, consists of, or consists essentially of a sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the second variable heavy chain comprises, consists of, or consists essentially of the sequence of SEQ ID NO: 1.

### 2.2.4 V_{L} Sequences

In some embodiments, the second variable light chain comprises, consists of, or consists essentially of a sequence having at least 70% sequence identity to SEQ ID NO: 16. In some embodiments, the second variable light chain comprises, consists of, or consists essentially of a sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the second variable light chain comprises, consists of, or consists essentially of the sequence of SEQ ID NO: 16.

2.2.5 V_{H}/V_{L} Pair

In some embodiments, the second variable heavy chain comprises, consists of, or consists essentially of the sequence of SEQ ID NO: 16 and the second variable light chain comprises, consists of, or consists essentially of the sequence of SEQ ID NO: 23.

2.2.6 scFv Sequence

In some embodiments, the CD3 binding domain comprises, consists of, or consists essentially of a sequence having at least 70% sequence identity to SEQ ID NO: 15. In some embodiments, the CD3 binding domain comprises, consists of, or consists essentially of a sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 15. In some embodiments, the CD3 binding domain comprises, consists of, or consists essentially of the sequence of SEQ ID NO: 15.

### 2.2.7 Linkers

In some embodiments, the CD3 binding domain comprises a second linker positioned between the second variable heavy chain and the second variable light chain.

In some embodiments, the second linker comprises, consists of, or consists essentially of a flexible linker. In some embodiments, the second linker comprises, consists of, or consists essentially of a glycine/serine-rich linker. In some embodiments, the glycine/serine-rich linker comprises (GGGGS)ₙ, where n is 1, 2, 3, 4, 5, 6, 7 or 8. In some embodiments, the second linker comprises, consists of, or consists essentially of the sequence of any one of SEQ ID NO: 38, SEQ ID NO: 42, SEQ ID NO: 43, SEQ ID NO: 45, SEQ ID NO: 46, SEQ ID NO: 47, SEQ ID NO: 50, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99. In some embodiments, the second linker comprises, consists of, or consists essentially of the sequence of SEQ ID NO: 38. Exemplary linkers are disclosed in Chen, et al., “Fusion Protein Linkers: Property, Design and Functionality,” Adv Drug Deliv Rev. 2013 Oct. 15 65(10):1357-1369, Brinkmann U. and Kontermann, Roland, “The making of bispecific antibodies,” MAbs 2017 9(2):182-212, U.S. Patent Application Publication No. 2019/0092818 and International Patent Application Publication No. WO 2017/106462.

2.2.8 Arrangement of V_{H} and V_{L}

In some embodiments, the second variable heavy chain and the first variable light chain are oriented in order from N- to C-terminus of the CD3 binding domain. In some embodiments, the second variable light chain and the second variable heavy chain are oriented in order from N- to C-terminus.

### 2.2.9 Format of CD3 Binding Domain

In some embodiments, the CD3 binding domain is an antibody. In some embodiments, the CD3 binding domain is a Fv, scFv, Fab, F(ab′)2, or scFv-Fc. In some embodiments, the CD3 binding domain is a scFv. In some embodiments, the CD3 binding domain is a humanized antibody or fragment thereof. In some embodiments, the CD3 binding domain is a chimeric antibody or fragment thereof.

In some embodiments, the second variable heavy chain is an IgA. In some embodiments, the Second variable heavy chain is an IgD. In some embodiments, the second variable heavy chain is an IgE. In some embodiments, the second variable heavy chain is an IgG. In some embodiments, the second variable heavy chain is an IgM. In some aspects, the second variable heavy chain is an IgG1. In some aspects, the second variable heavy chain is an IgG2. In some aspects, the second variable heavy chain is an IgG3. In some aspects, the second variable heavy chain is an IgG4. In some aspects, the second variable heavy chain is an IgA1. In some aspects, the second variable heavy chain is an IgA2.

In some embodiments, the second variable light chain is a kappa light chain. In some embodiments, the second variable light chain is a lambda light chain.

In some embodiments, the CD3 binding domain is a humanized antibody. In some embodiments, the CD3 binding domain is a chimeric antibody.

In some embodiments, the CD3 binding domain is an affinity matured antibody.

### 2.2.10 CDR Variants

In some embodiments, the CDRs of the CD3 binding domain have at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to those recited herein. In some embodiments, the CDRs of the CD3 binding domain differ by 1, 2 or 3 amino acid substitutions from the CDRs recited herein. In certain aspects, the substitutions are conservative substitutions.

### 2.3 Cytokine Domain

In some embodiments, the CD22xCD3 binding polypeptide further comprises a cytokine domain.

### 2.3.1 Cytokine

In some embodiments, the cytokine domain comprises, consists of, or consists essentially of a cytokine. In some embodiments, the cytokine is selected from the group consisting of IL-2, IL-7, IL-12, IL-15, IL-18, IL-21, IL-23, interferon-α, interferon-β, interferon-γ and tumor necrosis factor α or a mutant, variant or functional fragment thereof. In some embodiments, the cytokine is selected from the group consisting of IL-2, IL-12 and IL-15 or a mutant, variant or functional fragment thereof. In some embodiments, the cytokine can be a superkine, such as, by way of example but not limitation, the IL-2 disclosed in Merchant, et al., “Fine-tuned long-acting interleukin-2 superkine potentiates durable immune responses in mice and non-human primate,” Clin/Trans Cancer Immunotherapy 10(1):e003155 (2022).

In some embodiments, the cytokine comprises IL-2 or a mutant, variant or functional fragment thereof.

In some embodiments, the cytokine comprises IL-12, or a mutant, variant or functional fragment thereof. In some embodiments, the IL-12, mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 97 and SEQ ID NO: 98. In some embodiments, the IL-12, mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 97 and SEQ ID NO: 101. In some embodiments, the IL-12, mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 100 and SEQ ID NO: 98. In some embodiments, the IL-12, mutant, variant or functional fragment thereof comprises, consists of, consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 100 and SEQ ID NO: 101.

In some embodiments, the IL-12, mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 97 and SEQ ID NO: 98. In some embodiments, the IL-12, mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 97 and amino acids 57-253 of SEQ ID NO: 101. In some embodiments, the IL-12, mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to amino acids 23-329 of SEQ ID NO: 100 and SEQ ID NO: 98. In some embodiments, the IL-12, mutant, variant or functional fragment thereof comprises, consists of, consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to amino acids 23-329 of SEQ ID NO: 100 and amino acids 57-253 of SEQ ID NO: 101.

In some embodiments, the IL-12, mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 104. In some embodiments, the IL-12, mutant, variant or functional fragment thereof comprises, consists of, or consists essentially of the sequence of SEQ ID NO: 104.

In some embodiments, the cytokine comprises IL-15 or a mutant, variant or functional fragment thereof. In some embodiments, the IL-15, mutant, variant or functional fragment thereof comprises, consists of, or consists essentially of a sequence having at least 70% identity to SEQ ID NO: 34 or SEQ ID NO: 35. In some embodiments, the IL-15, mutant, variant or functional fragment thereof comprises, consists of, or consists essentially of a sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 34 or SEQ ID NO: 35. In some embodiments, the IL-15, mutant, variant or functional fragment thereof comprises, consists of, or consists essentially of the sequence of SEQ ID NO: 34. In some embodiments, the IL-15, mutant, variant or functional fragment thereof comprises, consists of, or consists essentially of the sequence of SEQ ID NO: 35. In some embodiments, the IL-15, mutant, variant or functional fragment thereof comprises a N72D, N72A, N72E, N72S, N72Y or N72P mutation at a position corresponding to position 72 of SEQ ID NO: 34. In some embodiments, the IL-15, mutant, variant or functional fragment thereof comprises, consists of, or consists essentially of the sequence of SEQ ID NO: 36, SEQ ID NO: 74, SEQ ID NO: 75, SEQ ID NO: 76, SEQ ID NO: 77 or SEQ ID NO: 78.

### 2.3.2 Sushi Domain

In some embodiments, the cytokine domain further comprises a sushi domain. In some embodiments, the sushi domain is the sushi domain of IL-15Ra, a mutant, variant or functional fragment thereof. In some embodiments, the sushi domain, mutant, variant or functional fragment thereof comprises, consists of, or consists essentially of a sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 32 or SEQ ID NO: 33. In some embodiments, the sushi domain comprises, consists of, or consists essentially of the sequence of SEQ ID NO: 32. In some embodiments, the sushi domain comprises, consists of, or consists essentially of the sequence of SEQ ID NO: 33. It should be understood that other sushi domains or fragment or mutants thereof can be used with different cytokines.

### 2.3.3 Linkers

In some embodiments, the sushi domain can be fused to the IL-15 cytokine. In some embodiments, the sushi domain can be fused to the IL-15 cytokine by a sixth linker. In some embodiments, the sixth linker comprises, consists of, or consists essentially of a flexible linker. In some embodiments, the sixth linker comprises, consists of, or consists essentially of a glycine/serine-rich linker. In some embodiments, the glycine/serine-rich linker comprises (GGGGS)ₙ, where n is 1, 2, 3, 4, 5, 6, 7 or 8. In some embodiments, the sushi domain can be fused to the IL-15 cytokine by a sixth linker selected from any one of SEQ ID NO: 38, SEQ ID NO: 42, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99. In some embodiments, the sushi domain can be fused to the IL-15 cytokine by a sixth linker comprising SEQ ID NO: 51 (G₃SG₄SG₃SG₄SLQ). Exemplary linkers are disclosed in Chen, et al., “Fusion Protein Linkers: Property, Design and Functionality,” Adv Drug Deliv Rev. 2013 Oct. 15 65(10):1357-1369, Brinkmann U. and Kontermann, Roland, “The making of bispecific antibodies,” MAbs 2017 9(2):182-212, U.S. Patent Application Publication No. 2019/0092818 and International Patent Application Publication No. WO 2017/106462.

### 2.3.4 Sushi-IL-15 Fusion

In some embodiments, the cytokine domain comprises a sushi-IL-15 fusion protein comprising a sushi domain and IL-15 or a mutant, variant or functional fragment thereof. In some embodiments, the sushi domain comprises the sequence of SEQ ID NO: 37.

### 2.4 Arrangement of Domains

In some embodiments, the CD22 binding domain is positioned on an N-terminal end of the cytokine domain and the CD3 binding domain is positioned on a C-terminal end of the cytokine domain. In some embodiments, the CD22 binding domain is positioned on a C-terminal end of the cytokine domain and the CD3 binding domain is positioned on an N-terminal end of the cytokine domain. In some embodiments, the CD22 binding domain, CD3 binding domain and cytokine domain are in order from N-terminus to C-terminus. In some embodiments, the CD3 binding domain, the CD22 binding domain and the cytokine domain are in order from N-terminus to C-terminus. In some embodiments, the cytokine binding domain, CD22 binding domain and CD3 binding domain are in order from N-terminus to C-terminus. In some embodiments, the cytokine binding domain, CD3 binding domain and CD22 binding domain are in order from N-terminus to C-terminus.

### 2.5 Linkers

In some embodiments, the CD3 binding domain is linked to the CD22 binding domain by a third linker. In some embodiments, the third linker comprises, consists of, or consists essentially of a flexible linker. In some embodiments, the third linker comprises, consists of, or consists essentially of a glycine/serine-rich linker. In some embodiments, the glycine/serine-rich linker comprises (GGGGS)ₙ, where n is 1, 2, 3, 4, 5, 6, 7 or 8. In some embodiments, the third linker comprises, consists of, or consists essentially of the sequence of any one of SEQ ID NO: 38, SEQ ID NO: 39, SEQ ID NO: 40, SEQ ID NO: 41, SEQ ID NO: 42, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99. In some embodiments, the third linker comprises, consists of, or consists essentially of the sequence of SEQ ID NO: 38. In some embodiments, the third linker comprises, consists of, or consists essentially of the sequence of SEQ ID NO: 39. In some embodiments, the third linker comprises, consists of, or consists essentially of the sequence of SEQ ID NO: 40. In some embodiments, the third linker comprises, consists of, or consists essentially of the sequence of SEQ ID NO: 41. Exemplary linkers are disclosed in Chen, et al., “Fusion Protein Linkers: Property, Design and Functionality,” Adv Drug Deliv Rev. 2013 Oct. 15 65(10):1357-1369, Brinkmann U. and Kontermann, Roland, “The making of bispecific antibodies,” MAbs 2017 9(2):182-212, U.S. Patent Application Publication No. 2019/0092818 and International Patent Application Publication No. WO 2017/106462. In some embodiments, the third linker can comprise a cysteine residue which can be capable of conjugation. In embodiments where the third linker comprises a cysteine, this residue can be used for conjugation, such as for an additional binding arm of the binding polypeptide such as, for example, a cytokine domain as described herein such as IL-15 or a sushi-IL-15 fusion.

In some embodiments, the CD22 binding domain is linked to the cytokine domain by a fourth linker. In some embodiments, the fourth linker comprises, consists of, or consists essentially of a flexible linker. In some embodiments, the fourth linker comprises, consists of, or consists essentially of a glycine/serine-rich linker. In some embodiments, the glycine/serine-rich linker comprises (GGGGS)ₙ, where n is 1, 2, 3, 4, 5, 6, 7 or 8. In some embodiments, the fourth linker comprises, consists of, or consists essentially of a sequence selected from any one of SEQ ID NO: 38, SEQ ID NOs: 42-51, SEQ ID NOs: 84-89, SEQ ID NOs: 92-94, and SEQ ID NO: 99. In some embodiments, the fourth linker comprises, consists of, or consists essentially of the sequence of SEQ ID NO: 46. In some embodiments, the CD3 binding domain is linked to the cytokine domain by a fifth linker. In some embodiments, the fifth linker comprises, consists of, or consists essentially of a flexible linker. In some embodiments, the fifth linker comprises, consists of, or consists essentially of a glycine/serine-rich linker. In some embodiments, the glycine/serine-rich linker comprises (GGGGS)ₙ, where n is 1, 2, 3, 4, 5, 6, 7 or 8. In some embodiments, the fifth linker comprises, consists of, or consists essentially of a sequence selected from any one of SEQ ID NO: 38, SEQ ID NOs: 42-51, SEQ ID NOs: 84-89, SEQ ID NOs: 92-94, and SEQ ID NO: 99. In some embodiments, the fifth linker comprises, consists of, or consists essentially of the sequence of SEQ ID NO: 47. It should be understood that any suitable linker can be used as a fourth linker or fifth linker including any one of SEQ ID NO: 38, SEQ ID NOs: 42-51 and 84-89. Exemplary linkers are disclosed in Chen, et al., “Fusion Protein Linkers: Property, Design and Functionality,” Adv Drug Deliv Rev. 2013 Oct. 15 65(10):1357-1369, Brinkmann U. and Kontermann, Roland, “The making of bispecific antibodies,” Mabs 2017 9(2):182-212, U.S. Patent Application Publication No. 2019/0092818 and International Patent Application Publication No. WO 2017/106462.

In bispecific antibodies, or multispecific binding proteins, the central linker between two scFvs can affect the performance of the binding protein. It has been suggested that a longer linker can be deleterious because it can disrupt the immune synapse and lead to less efficient killing.

In some embodiments, the CD22 binding domain can be linked to the CD3 binding domain by a linker where the cytokine domain is at an N-terminal end or a C-terminal end of the binding polypeptide. Any suitable linkers disclosed herein can be used between the CD3 and CD22 binding domains including those described as the third linker herein. Exemplary linkers are disclosed in Chen, et al., “Fusion Protein Linkers: Property, Design and Functionality,” Adv Drug Deliv Rev. 2013 Oct. 15 65(10):1357-1369, Brinkmann U. and Kontermann, Roland, “The making of bispecific antibodies,” Mabs 2017 9(2):182-212, U.S. Patent Application Publication No. 2019/0092818 and International Patent Application Publication No. WO 2017/106462. In some embodiments, the cytokine domain can be conjugated via a cysteine residue in the third linker between the CD22 binding domain and the CD3 binding domain. In some embodiments, where the CD22xCD3 binding polypeptide includes a cytokine domain, none of the linkers comprise a cysteine residue.

### 2.6 Full-Length Sequences

In some embodiments, the CD22xCD3 binding polypeptide comprises, consists of, or consists essentially of SEQ ID NO: 31. In some embodiments, the CD22xCD3 binding polypeptide comprises, consists of, or consists essentially of SEQ ID NO: 52. In some embodiments, the CD22xCD3 binding polypeptide comprises, consists of, or consists essentially of SEQ ID NO: 57. In some embodiments, the CD22xCD3 binding polypeptide comprises, consists of, or consists essentially of SEQ ID NO: 67. In some embodiments, the CD22xCD3 binding polypeptide comprises, consists of, or consists essentially of SEQ ID NO: 79. In some embodiments, the CD22xCD3 binding polypeptide comprises, consists of, or consists essentially of SEQ ID NO: 91. In some embodiments, the CD22xCD3 binding polypeptide comprises, consists of, or consists essentially of SEQ ID NO: 96. In some embodiments, the CD22xCD3 binding polypeptide comprises, consists of, or consists essentially of SEQ ID NO: 103. In some embodiments, the CD22xCD3 binding polypeptide comprises, consists of, or consists essentially of SEQ ID NO: 96 with a glycine inserted between Gly 125 and Ser126. In some embodiments, the CD22xCD3 binding polypeptide comprises, consists of, or consists essentially of SEQ ID NO: 103 with a glycine inserted between Gly 147 and Ser148.

In some embodiments, the CD22xCD3 binding polypeptide comprises, consists of, or consists a sequence having at least 70%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 31. In some embodiments, the CD22xCD3 binding polypeptide comprises, consists of, or consists a sequence having at least 70%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 52. In some embodiments, the CD22xCD3 binding polypeptide comprises, consists of, or consists a sequence having at least 70%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 57. In some embodiments, the CD22xCD3 binding polypeptide comprises, consists of, or consists a sequence having at least 70%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 67. In some embodiments, the CD22xCD3 binding polypeptide comprises, consists of, or consists a sequence having at least 70%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 79. In some embodiments, the CD22xCD3 binding polypeptide comprises, consists of, or consists a sequence having at least 70%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 91. In some embodiments, the CD22xCD3 binding polypeptide comprises, consists of, or consists a sequence having at least 70%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 96. In some embodiments, the CD22xCD3 binding polypeptide comprises, consists of, or consists a sequence having at least 70%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 103.

### 2.7 Conjugates

In some embodiments, the CD22xCD3 binding polypeptide further comprises a payload. In some embodiments, the payload is a drug payload, such as a cytotoxic payload. In some embodiments, the payload is selected from the group consisting of an auristatin such as monomethyl auristatin E (MMAE) or monomethyl auristatin F (MMAF), a maytansine such as a maytansinoid such as DM1 or DM4, a calicheamicin, a duocarymycin such as CC-1065 or duocarmycin SA, a pyrrolobenzodiazepine dimer, an amatoxin such as α-amaitin, a protein toxin or immunotoxin such as Pseudomonas endotoxin or diphtheria toxin, an antibiotic such as 4-dimethylamino piperidino-hydoxybenzoxazino rifamycin, an enzyme such as β-glucuronidase, an anthracycline such as daunorubicin, doxorubicin or epirubicin, a radionuclide, camptothecin, and exatecan. In some embodiments, the payload is selected form the group consisting of monomethyl auristatin E, saporin, calicheamicin, Pseudomonas endotoxin A or a truncated fragment thereof, diphtheria toxin or a fragment thereof, and a deglycosylated ricin such as a deglycosylated ricin A chain. In certain embodiments, the payload can be conjugated at a cysteine residue. In certain embodiments, the payload can be conjugated to the CD22xCD3 binding polypeptide at a cysteine in a SGGCGGS linker (SEQ ID NO: 41) present in the CD22xCD3 binding polypeptide. By way of example, but not limitation, the payload can be conjugated at Cys251 of SEQ ID NO: 31 or a corresponding position in a BiTE-197 construct. For example, maleimide chemistry can be used to attach a molecule via thioether bond at a cysteine residue.

### 3. Structure of Multispecific Polypeptides

In some embodiments, a multispecific binding polypeptide can include a first binding domain which specifically bind to a first target, a second binding domain which specifically binds to a second target, and a cytokine domain comprising a cytokine or a mutant, variant or functional fragment thereof.

### 3.1 First and Second Binding Domains

In some embodiments, the first binding domain comprises an antibody or fragment thereof. In some embodiments, the second binding domain comprises an antibody or fragment thereof.

In some embodiments, the first binding domain is a scFv, Fab, F(ab′)2, or scFv-Fc. In some embodiments, the first binding domain is a scFv. In some embodiments, the first binding domain is a humanized antibody or fragment thereof. In some embodiments, the first binding domain is a chimeric antibody or fragment thereof. In some embodiments, the second binding domain is a human antibody or fragment thereof. In some embodiments, the first binding domain is a monoclonal antibody. In some embodiments, the first binding domain is a polyclonal antibody.

In some embodiments, the second binding domain is a scFv, Fab, F(ab′)2, or scFv-Fc. In some embodiments, the second binding domain is a scFv. In some embodiments, the second binding domain is a humanized antibody or fragment thereof. In some embodiments, the second binding domain is a chimeric antibody or fragment thereof. In some embodiments, the second binding domain is a human antibody or fragment thereof. In some embodiments, the second binding domain is a monoclonal antibody. In some embodiments, the second binding domain is a polyclonal antibody.

In some embodiments, the first binding domain or second binding domain selectively bind to a target selected from the group consisting of CD3, CD19, CD20, CD22, BCMA and CD33, where at least one of the first target and second target is CD3.

In some embodiments, the first binding domain comprises, consists of, or consists essentially of a T-cell engaging domain.

In some embodiments, the second binding domain specifically binds a tumor antigen.

### 3.1.1 First Binding Domain

In some embodiments, the first binding domain comprises, consists of, or consists essentially of a first variable heavy chain and a first variable light chain.

In some embodiments, the first binding domain can further comprise a first linker positioned between the first variable heavy chain and the first variable light chain. In some embodiments, the first linker can be as described herein above.

In some embodiments, the first variable heavy chain and the first variable light chain are oriented in order from N- to C-terminus of the first binding domain. In some embodiments, the first variable light chain and the first variable heavy chain are oriented in order from N- to C-terminus.

In some embodiments, the first binding domain is an antibody. In some embodiments, the first binding domain is a Fv, scFv, Fab, F(ab′)2, or scFv-Fc. In some embodiments, the first binding domain is a scFv. In some embodiments, the first binding domain is a humanized antibody or fragment thereof. In some embodiments, the first binding domain is a chimeric antibody or fragment thereof.

In some embodiments, the first variable heavy chain is an IgA. In some embodiments, the First variable heavy chain is an IgD. In some embodiments, the first variable heavy chain is an IgE. In some embodiments, the first variable heavy chain is an IgG. In some aspects, the first variable heavy chain is an IgG1. In some aspects, the first variable heavy chain is an IgG2. In some aspects, the first variable heavy chain is an IgG3. In some aspects, the first variable heavy chain is an IgG4. In some aspects, the first variable heavy chain is an IgA1. In some aspects, the first variable heavy chain is an IgGA2.

In some embodiments, the first variable light chain is a kappa light chain. In some embodiments, the first variable light chain is a lambda light chain.

In some embodiments, the first binding domain is a humanized antibody. In some embodiments, the first binding domain is a chimeric antibody.

In some embodiments, the first binding domain is an affinity matured antibody.

In some embodiments, the first binding domain specifically binds T-cells. In some embodiments, the first binding domain comprises a CD3 binding domain disclosed herein.

In some embodiments, the second target is selected from CD3, CD19, CD20, CD22, BCMA, and CD33, where at least one of the first target and second target is CD3.

### 3.1.2 Second Binding Domain

In some embodiments, the second binding domain comprises, consists of, or consists essentially of a second variable heavy chain and a second variable light chain.

In some embodiments, the second binding domain comprises, consists of, or consists essentially of a second variable heavy chain and a second variable light chain.

In some embodiments, the second binding domain can further comprise a second linker positioned between the second variable heavy chain and the second variable light chain. In some embodiments, the second linker can be as described herein above.

In some embodiments, the second variable heavy chain and the second variable light chain are oriented in order from N- to C-terminus of the second binding domain. In some embodiments, the second variable light chain and the second variable heavy chain are oriented in order from N- to C-terminus.

In some embodiments, the second binding domain is an antibody. In some embodiments, the second binding domain is a Fv, scFv, Fab, F(ab′)2, or scFv-Fc. In some embodiments, the second binding domain is a scFv. In some embodiments, the second binding domain is a humanized antibody or fragment thereof. In some embodiments, the second binding domain is a chimeric antibody or fragment thereof.

In some embodiments, the second variable heavy chain is an IgA. In some embodiments, the Second variable heavy chain is an IgD. In some embodiments, the second variable heavy chain is an IgE. In some embodiments, the second variable heavy chain is an IgG. In some aspects, the second variable heavy chain is an IgG1. In some aspects, the second variable heavy chain is an IgG2. In some aspects, the second variable heavy chain is an IgG3. In some aspects, the second variable heavy chain is an IgG4. In some aspects, the second variable heavy chain is an IgA1. In some aspects, the second variable heavy chain is an IgGA2.

In some embodiments, the second variable light chain is a kappa light chain. In some embodiments, the second variable light chain is a lambda light chain.

In some embodiments, the second binding domain is a humanized antibody. In some embodiments, the second binding domain is a chimeric antibody.

In some embodiments, the second binding domain is an affinity matured antibody.

In some embodiments, the second binding domain specifically binds T-cells. In some embodiments, the second binding domain comprises a CD3 binding domain disclosed herein.

In some embodiments, the second target is selected from CD3, CD19, CD20, CD22, BCMA and CD33, where at least one of the first target and second target is CD3.

### 3.2 Cytokine Domain

### 3.2.1 Cytokine

In some embodiments, the cytokine domain comprises, consists of, or consists essentially of a cytokine. In some embodiments, the cytokine is selected from the group consisting of IL-2, IL-7, IL-12, IL-15, IL-18, IL-21, IL-23, interferon-α, interferon-β, interferon-γ and tumor necrosis factor α or a mutant, variant or functional fragment thereof. In some embodiments, the cytokine is selected from the group consisting of IL-2, IL-12 and IL-15 or a mutant, variant or functional fragment thereof. In some embodiments, the cytokine can be a superkine, such as, by way of example but not limitation, the IL-2 disclosed in Merchant, et al., “Fine-tuned long-acting interleukin-2 superkine potentiates durable immune responses in mice and non-human primate,” Clin/Trans Cancer Immunotherapy 10(1):e003155 (2022).

In some embodiments, the cytokine comprises IL-2 or a mutant, variant or functional fragment thereof.

In some embodiments, the cytokine comprises IL-12 or a mutant, variant or functional fragment thereof. In some embodiments, the IL-12, mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 97 and SEQ ID NO: 98. In some embodiments, the IL-12 mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 97 and SEQ ID NO: 101. In some embodiments, the IL-12 mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 100 and SEQ ID NO: 98. In some embodiments, the IL-12 mutant, variant or functional fragment thereof comprises, consists of, consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 100 and SEQ ID NO: 101.

In some embodiments, the IL-12, mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 97 and SEQ ID NO: 98. In some embodiments, the IL-12 mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 97 and amino acids 57-253 of SEQ ID NO: 101. In some embodiments, the IL-12 mutant, variant or functional fragment thereof comprises, consists of, or consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to amino acids 23-329 of SEQ ID NO: 100 and SEQ ID NO: 98. In some embodiments, the IL-12 mutant, variant or functional fragment thereof comprises, consists of, consists essentially of a sequence having at least 70%, at least 80%, at least 85%, at least 90%, at least 91%, at least 92%, at least 93%, at least 94%, at least 95%, at least 96%, at least 97%, at least 98%, at least 99% or 100% sequence identity to amino acids 23-329 of SEQ ID NO: 100 and amino acids 57-253 of SEQ ID NO: 101.

In some embodiments, the cytokine comprises IL-15 or a mutant, variant or functional fragment thereof. In some embodiments, the IL-15, mutant, variant or functional fragment thereof comprises, consists of, or consists essentially of a sequence having at least 70% identity to SEQ ID NO: 34 or SEQ ID NO: 35.

In some embodiments, the IL-15, mutant, variant or functional fragment thereof comprises, consists of, or consists essentially of a sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 34 or SEQ ID NO: 35. In some embodiments, the IL-15, mutant, variant or functional fragment thereof comprises, consists of, or consists essentially of the sequence of SEQ ID NO: 34. In some embodiments, the IL-15, mutant, variant or functional fragment thereof comprises, consists of, or consists essentially of the sequence of SEQ ID NO: 35. In some embodiments, the IL-15, mutant, variant or functional fragment thereof comprises a N72D, N72A, N72E, N72S, N72Y or N72P mutation at a position corresponding to position 72 of SEQ ID NO: 34. In some embodiments, the IL-15, mutant, variant or functional fragment thereof comprises, consists of, or consists essentially of the sequence of SEQ ID NO: 36.

### 3.2.2 Sushi Domain

In some embodiments, the cytokine domain further comprises a sushi domain. In some embodiments, the sushi domain is the sushi domain of IL-15Ra, a mutant, variant or functional fragment thereof. In some embodiments, the sushi domain, mutant, variant or functional fragment thereof comprises, consists of, or consists essentially of a sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 32 or SEQ ID NO: 33. In some embodiments, the sushi domain comprises, consists of, or consists essentially of the sequence of SEQ ID NO: 32. In some embodiments, the sushi domain comprises, consists of, or consists essentially of the sequence of SEQ ID NO: 33.

### 3.2.3 Linkers

In some embodiments, the sushi domain can be fused to the IL-15 cytokine. In some embodiments, the sushi domain can be fused to the IL-15 cytokine by a sixth linker. In some embodiments, the sixth linker comprises, consists of, or consists essentially of a flexible linker. In some embodiments, the sixth linker comprises, consists of, or consists essentially of a glycine/serine-rich linker. In some embodiments, the glycine/serine-rich linker comprises (GGGGS)ₙ, where n is 1, 2, 3, 4, 5, 6, 7 or 8. In some embodiments, the sushi domain can be fused to the IL-15 cytokine by a sixth linker selected from any one of SEQ ID NO: 38, SEQ ID NO: 42, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99. In some embodiments, the sushi domain can be fused to the IL-15 cytokine by a sixth linker comprising SEQ ID NO: 51 (G₃SG₄SG₃SG₄SLQ). Exemplary linkers are disclosed in Chen, et al., “Fusion Protein Linkers: Property, Design and Functionality,” Adv Drug Deliv Rev. 2013 Oct. 15 65(10):1357-1369, Brinkmann U. and Kontermann, Roland, “The making of bispecific antibodies,” MAbs 2017 9(2):182-212, U.S. Patent Application Publication No. 2019/0092818 and International Patent Application Publication No. WO 2017/106462.

### 3.2.4 Sushi-IL-15 Fusion

In some embodiments, the cytokine domain comprises a sushi-IL-15 fusion protein comprising a sushi domain and IL-15 or a mutant, variant or functional fragment thereof. In some embodiments, the sushi domain comprises the sequence of SEQ ID NO: 37.

### 3.3 Arrangement of Domains

In some embodiments, the first binding domain is positioned on an N-terminal end of the cytokine domain and the second binding domain is positioned on a C-terminal end of the cytokine domain. In some embodiments, the first binding domain is positioned on a C-terminal end of the cytokine domain and the second binding domain is positioned on an N-terminal end of the cytokine domain. In some embodiments, the first binding domain, second binding domain and cytokine domain are in order from N-terminus to C-terminus. In some embodiments, the second binding domain, the first binding domain and the cytokine domain are in order from N-terminus to C-terminus. In some embodiments, the cytokine binding domain, first binding domain and second binding domain are in order from N-terminus to C-terminus. In some embodiments, the cytokine binding domain, second binding domain and first binding domain are in order from N-terminus to C-terminus. In some embodiments, the cytokine domain can be conjugated via a cysteine residue in the third linker between the first binding domain and second binding domain. In some embodiments, where the CD22xCD3 binding polypeptide includes a cytokine domain, none of the linkers comprise a cysteine residue.

### 3.4 Linkers

In some embodiments, the first binding domain, second binding domain and the cytokine can each independently be linked to one another by any suitable linkers. In some embodiments, each linker is independently selected from the linkers disclosed herein. Exemplary linkers are disclosed in Chen, et al., “Fusion Protein Linkers: Property, Design and Functionality,” Adv Drug Deliv Rev. 2013 Oct. 15 65(10):1357-1369, Brinkmann U. and Kontermann, Roland, “The making of bispecific antibodies,” MAbs 2017 9(2):182-212, U.S. Patent Application Publication No. 2019/0092818 and International Patent Application Publication No. WO 2017/106462.

In some embodiments, the linker between a binding domain and the cytokine domain can comprise at least one cysteine residue capable of conjugation.

In some embodiments, the first binding domain can be linked to the cytokine domain by a fourth linker as described herein. In some embodiments, the second binding domain can be linked to the cytokine domain by a fifth linker as described herein.

In some embodiments, a binding polypeptide of the present disclosure can further include a His tag, such as a 6xHis tag. In some embodiments, the His tag is fused to the binding polypeptide by a linker such as GGS (SEQ ID NO: 53).

### 3.5 Conjugates

In some embodiments, the multispecific binding polypeptide further comprises a payload. In some embodiments, the payload is a drug payload, such as a cytotoxic payload. In some embodiments, the payload is selected from the group consisting of an auristatin such as monomethyl auristatin E (MMAE) or monomethyl auristatin F (MMAF), a maytansine such as a maytansinoid such as DM1 or DM4, a calicheamicin, a duocarymycin such as CC-1065 or duocarmycin SA, a pyrrolobenzodiazepine dimer, an amatoxin such as α-amaitin, a protein toxin or immunotoxin such as Pseudomonas endotoxin or diphtheria toxin, an antibiotic such as 4-dimethylamino piperidino-hydoxybenzoxazino rifamycin, an enzyme such as β-glucuronidase, an anthracycline such as daunorubicin, doxorubicin or epirubicin, a radionuclide, camptothecin, and exatecan. In some embodiments, the payload is selected form the group consisting of monomethyl auristatin E, saporin, calicheamicin, Pseudomonas endotoxin A or a truncated fragment thereof, diphtheria toxin or a fragment thereof, and a deglycosylated ricin such as a deglycosylated ricin A chain.

In certain embodiments, the payload can be conjugated at a cysteine residue. In certain embodiments, the payload can be conjugated to the multispecific binding polypeptide at a cysteine in a SGGCGGS linker (SEQ ID NO: 41) present in the multispecific binding polypeptide. For example, maleimide chemistry via reduction of a disulfide bond can be used to generate an attachment point at a cysteine residue.

### 4. Vectors/Host Cells/Pharmaceutical Compositions

In some embodiments, a polynucleotide encoding a CD22xCD3 binding polypeptide or multispecific binding polypeptide disclosed herein is provided.

In some embodiments, an expression vector comprising the polynucleotide encoding a CD22xCD3 binding polypeptide or multispecific binding polypeptide disclosed herein is provided.

For recombinant production of the antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety. In some embodiments, the nucleic acid may be synthesized and cloned into a vector using methods known in the art.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells.

The culture conditions, such as temperature, pH, and the like, are those previously used with the host cell selected for expression, and will be apparent to the ordinarily skilled artisan.

In some embodiments, a host cell comprising the polynucleotide or expression vector comprising the polynucleotide encoding a CD22xCD3 binding polypeptide or multispecific binding polypeptide disclosed herein is provided.

### 5. Methods of Manufacturing

In some embodiments, a method for producing a binding polypeptide of the present disclosure can include the step of incubating a host cell of comprising the polynucleotide encoding the binding polypeptide to yield the binding polypeptide.

### 6. Pharmaceutical Compositions and Methods of Treatment

### 6.1 Pharmaceutical Compositions

Any of the CD22xCD3 binding polypeptides or multispecific binding proteins of the present disclosure can be formulated in any appropriate pharmaceutical composition and administered by any suitable route of administration. Suitable routes of administration include, but are not limited to, inhalation, intraarterial, intradermal, intramuscular, intraperitoneal, intravenous, nasal, parenteral, pulmonary, and subcutaneous routes.

The pharmaceutical composition can comprise one or more pharmaceutical excipients and can be used to treat a disease or condition. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6^{th} Ed. (2009), incorporated by reference in its entirety.

The binding polypeptides described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with a tri-functional engager molecule without causing any undesirable biological effects or interacting in a deleterious manner with any of the other components of the pharmaceutical composition in which it is contained.

### 6.2 Methods of Treatment

In some embodiments, a method for treating cancer in a subject in need thereof comprises the step of administering a therapeutically effective amount of a binding polypeptide of this disclosure to the subject. In some embodiments, the cancer can be any B-cell cancer. In some embodiments, the cancer can be selected from the group consisting of prostate cancer, lung cancer, colon cancer, rectum cancer, urinary bladder cancer, melanoma, kidney cancer, renal cancer, oral cavity cancer, pharynx cancer, pancreas cancer, uterine cancer, thyroid cancer, skin cancer, head and neck cancer, cervical cancer, breast cancer, ovarian cancer, sarcoma, and hematopoietic cancer. In some embodiments, the cancer is acute lymphocytic leukemia. In some embodiments, the cancer is chronic lymphocytic leukemia (CLL). In some embodiments, the cancer is a B cell lymphoma, such as, by way of example but not limitation, diffuse large B-cell lymphoma (DLBCL), follicular lymphoma, mantle cell lymphoma (MCL), marginal zone lymphoma, Burkitt lymphoma, lymphoplasmacytic lymphoma (Waldenstom macroglobulinemia), hairy cell leukemia, primary central nervous system lymphoma, and primary intracocular lymphoma. In some embodiments, the cancer is non-Hodgkin's lymphoma.

In some embodiments, the methods provided herein further include administering the binding polypeptide prior to, simultaneously with, or following chemotherapy, surgical resection of a tumor, or radiation therapy. In some embodiments, the chemotherapy includes altretamine, amsacrine, L-asparaginase, colaspase, bleomycin, busulfan, capecitabine, carboplatin, carmustine, chlorambucil, cisplatin, cladribine, cyclophosphamide, cytophosphane, cytarabine, dacarbazine, dactinomycin, daunorubicin, docetaxel, doxorubicin, epirubicin, etoposide, fluorouracil, fludarabine, fotemustine, ganciclovir, gemcitabine, hydroxyurea, idarubicin, ifosfamaide, irinotecan, lomustine, melphalan, mercaptopurine, methotrexate, mitoxantrone, mitomycin C, nimustine, oxaliplatin, paclitaxel, pemetrexed, procarbazine, raltitrexed, temozolomide, teniposide, tioguanine, thiotepa, topotecan, vinblastine, vincristine, vindesine, and vinorelbine.

In some embodiments, the binding polypeptide is administered prior to, simultaneously with, or following therapy with a non-CD22-targeting B-cell therapeutic. In some embodiments, the binding polypeptide is administered prior to, simultaneously with, or following blinatumomab therapy. In some embodiments, the binding polypeptide is administered prior to, simultaneously with, or following rituximab therapy.

In some embodiments, the binding polypeptide is administered prior to, simultaneously with, or following adoptive cell therapy. In some embodiments, the binding polypeptide is administered prior to, simultaneously with, or following immunotherapy including CAR T therapy.

The amount of the antibody or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In some embodiments, the amount of the binding polypeptide administered can be between about 100 ng/Kg to about 50 mg/Kg of the subject's body weight, although a dose can be outside of this range. In some embodiments, the method can include administering sufficient amounts of the binding polypeptide to provide a dose of from about 10 μg/Kg to about 5 mg/Kg of the subject's body weight, for example, a dose of from about 100 μg/Kg to about 1 mg/Kg.

The dose can be administered on any suitable schedule, such as once, twice, thrice or four times weekly. One of skill in the art can adjust the dosage and dosing schedule using techniques known in the art and based on patient, disease, and other information.

In some embodiments, the dosing can be separated by about 1 day, about 2 days, about 3 days, about 5 days, about 10 days, about 15 days, about 30 days, about 45 days, about 2 months, about 75 days, about 3 months, or about 6 months. In some embodiments, administration can be repeated and may be separated by about 1 day, about 2 days, about 3 days, about 5 days, about 10 days, about 15 days, about 30 days, about 45 days, about 2 months, about 75 days, about 3 months, or about 6 months.

### 6.2.1. Combination Therapies

In some embodiments, methods of treatment of the present disclosure can further comprise administering to the subject a therapeutically effective amount of an antibody or fragment thereof, an antibody-drug conjugate (ADC), or an additional therapeutic agents such as other antibodies, chemotherapy cellular therapies (e.g., CAR T therapies including axicabtagene, lisocabtagene, brexucabtagene, Tisagenlecleucel), and immune modulators (e.g., lenalidomide, iberdomide, golcadamide, mezigdomide).

In some embodiments, the binding polypeptide is administered prior to, simultaneously with, or following antibody, ADC or other therapeutic treatments.

In some embodiments, the antibody or fragment thereof, comprises blinatumomab, an anti-CD19 antibody or fragment thereof, an anti-CD20 antibody or fragment thereof (such as rituximab or obinutuzumab), an anti-CD22 antibody or fragment thereof (such as epratuzumab), or an anti-B-cell maturation antigen (BCMA) antibody or fragment thereof.

In some embodiments, the antibody-drug conjugate comprises an anti-CD19, anti-CD20, anti-CD22 or anti-BCMA antibody or fragment thereof and a drug moiety selected from the group consisting of an auristatin such as monomethyl auristatin E (MMAE) or monomethyl auristatin F (MMAF), a maytansine such as a maytansinoid such as DM1 or DM4, a calicheamicin, a duocarymycin such as CC-1065 or duocarmycin SA, a pyrrolobenzodiazepine dimer, an amatoxin such as α-amaitin, a protein toxin or immunotoxin such as Pseudomonas endotoxin or diphtheria toxin, an antibiotic such as 4-dimethylamino piperidino-hydoxybenzoxazino rifamycin, an enzyme such as β-glucuronidase, an anthracycline such as PNU-159682, daunorubicin, doxorubicin or epirubicin, a radionuclide, camptothecin, and exatecan. In some embodiments, the drug moiety is selected form the group consisting of monomethyl auristatin E, saporin, calicheamicin, Pseudomonas endotoxin A or a truncated fragment thereof, diphtheria toxin or a fragment thereof, and a deglycosylated ricin such as a deglycosylated ricin A chain. In some embodiments, the drug moiety is selected from monomethyl auristatin E, saporin, calicheamicin, Pseudomonas endotoxin A or a truncated fragment thereof, diphtheria toxin or a fragment thereof, and a deglycosylated ricin A chain. In some embodiments, the ADC is inotuzumab ozogamici. In some embodiments, the ADC is moxetumomab pasudotox-tdfx. In some embodiments, the ADC is DT2219. In some embodiments, the ADC is IgG-RFB4-SMPT-dgA.

In some embodiments, the methods of treatment of the present disclosure can further comprise administering a therapeutically effective amount of a radioimmunoconjugate. In some embodiments, the radioimmunoconjugate is selected from the group consisting of 90Y-epratuzumab tetraxetan and BAY1862864.

In some embodiments, methods of treatment of the present disclosure can further comprise administering to the subject a therapeutically effective amount an engineered cell therapy comprising chimeric antigen receptor (CAR)-T cells.

In some embodiments, the CAR-T cells are engineered to target antigens commonly present on B-cell cancers such as, by way of example, but not limitation, CD19, CD20, CD22 and BCMA.

In some embodiments, the CAR-T cells are anti-CD19 CAR-T cells. In some embodiments, the CAR-T cells are anti-CD20 CAR-T cells. In some embodiments, the CAR-T cells are anti-CD22 CAR-T cells. In some embodiments, the CAR-T cells are anti-BCMA CAR-T cells.

In some embodiments, the CAR-T cells are allogeneic. In some embodiments, the CAR-T cells are autologous.

In some embodiments, the CAR-T cells can be administered at a dose from about 10⁴ to about 10⁹ cells/kg body weight, preferably about 10⁵ to about 10⁶ cells/kg body weight, including all integer values within those ranges and any range therebetween.

In some embodiments, the CAR-T cells can be administered prior to, simultaneously with, or after administration of the binding polypeptide of the present disclosure.

### 7. Affinity Purification Reagents

In some embodiments, the CD22xCD3 binding polypeptides or multispecific binding polypeptides disclosed herein can be used for affinity purification. For example, the binding polypeptides can be immobilized on a surface such as a resin using methods known in the art. The immobilized binding polypeptides can be contacted with a sample containing an antigen or protein of interest and the support washed to remove substantially all material in the sample except for the antigen or protein of interest bound to the immobilized binding polypeptides. Finally, the support is washed with another suitable solvent, such as glycine buffer, pH 5.0 or glycine buffer, pH 3 to 4, that will release the antigen protein from the binding polypeptide. In some embodiments, the antigen protein is CD3. In some embodiments, the antigen protein is CD22.

### 8. Diagnostic Applications

In some embodiments, the CD22xCD3 binding polypeptides or multispecific binding polypeptides can be used in diagnostic applications.

In some embodiments, the CD22xCD3 binding polypeptide or multispecific binding polypeptides can be useful in CD3 assays. In some aspects, the CD22xCD3 binding polypeptide or multispecific binding polypeptides can be used to detect CD3 in various cells and tissues. These assays may be useful for the diagnosis or prognosis of disease such as cancer.

In some embodiments, the CD22xCD3 binding polypeptide or multispecific binding polypeptides can be useful in CD22 assays. In some aspects, the CD22xCD3 binding polypeptide or multispecific binding polypeptides can be used to detect CD22 in various cells and tissues. These assays may be useful for the diagnosis or prognosis of disease such as cancer.

### 9. Kits

In some embodiments, the CD22xCD3 binding polypeptide or multispecific binding polypeptide can be provided as a component of a kit, i.e., a packaged combination of reagents in predetermined amounts with instructions for use in performing a procedure. In some embodiments, the procedure is a diagnostic assay. In some embodiments, the procedure is a method of treatment.

It should be understood that in embodiments of the present disclosure, the binding polypeptides and multispecific binding polypeptides of the present disclosure can include one more additional domains. The additional domains can be any domain deemed suitable by the person of skill. In certain embodiments, one or more additional domains are cytokine domains. In certain embodiment, one or more additional domains are other than cytokine domains. By way of example, but not limitation, a CD22xCD3 binding polypeptide can comprise a third domain that is a cytokine domain or a different third domain. By way of further example, but not limitation, the cytokine domain in any of the binding polypeptides and multispecific binding polypeptides disclosed herein can be substituted by a different third domain.

It should also be understood that linkers of the binding polypeptides of the present disclosure can also be modified such as by lacking a glycine residue and still be within the scope of the present disclosure.

In some embodiments, a third domain of a binding polypeptide of the present disclosure can include a T cell activating domain which acts through ‘Signal 2’ or ‘Signal 3’ agonism. Some exemplary activating ligands include B7-1 (CD80), B7-2 (CD86), Inducible T-cell COStimulator ligand (ICOS-L), 4-1BB ligand and OX40 ligand. Additionally, examples of ‘signal 3’ activators include cytokines, as well as molecules that bind and activate T cells via signal 3. Examples of signal 3 activators include IL-2, IL-7, IL-12, IL-15, IL-18, IL-21, IL-23, interferon-α, interferon-β, interferon-γ and tumor necrosis factor α or a mutant, variant or functional fragment thereof. In certain embodiments, the third domain is positioned between the first binding domain and the second binding domain.

### EXAMPLES

For the following examples, unless otherwise specified, the following cell lines were obtained: Raji (ATCC, CCL-86), NALM-6 (DSMZ, ACC 128), K562 (ATCC, CCL-243), U2973 (DSMZ, ACC 642), and Expi293F cells from Expi293 Expression System Kit (Gibco, A14635). RPMI (Gibco, 21-870-076) was supplemented with Fetal Bovine Serum (FBS) (Gibco, A3160402) to 10%, Glutamax (Gibco, 35050061) to 20 mM, sodium pyruvate (Gibco, 11360070) to 1 mM, HEPES buffer (Gibco, 15630080) to 10 mM, 4.5 g/L glucose, and 5 mL of penicillin/streptomycin (Gibco, 15140122) and was used to maintain Raji, NALM-6, and U2973 cells. DMEM (Gibco, 11960044) media was supplemented with 10% FBS and pen/strep and was used for maintaining K562 cells. Expi293F media was used for maintaining Expi293F cells according to manufacturer's protocol. The following antibodies were obtained for analysis via flow cytometry: anti-CD3-A488 (Clone OKT3, eBioscience), anti-His-PE antibody (clone J095G46 Biolegend), anti-CD22-BV421 antibody (Clone HIB22 Biolegend), anti-CD69-APC Antibody, (Clone FN50 Biolegend), and anti-CD25-BV650 antibody (Clone PC61, Biolegend). Zombie NIR fixable viability kit (Biolegend, 423106) and CFSE (eBioscience, 65-0850-84) were used in live/dead staining for cellular cytotoxicity analysis. Blinatumomab (Blincyto®) was obtained from Amgen (Thousand Oaks, CA). IL-2 (rhIL-2, Biological Resources Branch, NCI, Frederick, MD) was used selectively for in vitro assays.

Graphical and statistical analysis were performed using GraphPad Prism 9.1 (GraphPad Software Inc. San Diego, CA). In vitro cytotoxicity assays were analyzed using unpaired students t-test with Welch correction. Significance between T cells activated with BiTE 197 or blina were determined using 1-way ANOVA with Tukey's multiple comparison. In vitro assays were repeated at least twice (2 biological replicates) with 2-3 technical replicates to confirm significance of results. 3 separate PBMC donors were used to evaluate cytotoxicity and 2 donors were used to evaluate T cell activation markers. Figures were created from a representative replicate. Log rank mantel-cox significance test was performed to determine whether treatment groups in vivo were significantly different.

### Example 1: Generation of Bispecific and Tri-Functional Constructs and Purification

Hexahistadine (6xHis) or human influenza hemagglutinin (HA) tagged αCD22xαCD3 BiTE constructs were designed with two single chain variable regions (scFv) for αCD3 and αCD22 with various linker sequences to determine an ideal scaffolding format. The construction of all BiTEs was based upon a similar protocol as described by Loffler et Al. (2000) A recombinant bi-specific single-chain antibody CD19XCD3, induces rapid and high lymphoma-directed cytotoxicity by unstimulated T lymphocytes Blood 95:6. The αCD3 binding amino acid sequence was derived from the heavy and light chains of the well-characterized CD3 antibody TR66, with a 4xGGGGS linker between the two chains. The αCD22 sequence was derived from a well characterized αCD22 blocking antibody, HB22.7. Proposed constructs (BiTE #197 and BiTE #198) with the same binding domain sequences were generated with different orientations of the linker position, composition, and 6xHis or HA tag. Constructs were cloned into pcDNA3.1+ vector (GenScript) for expression in Expi293F cells and secretion in cell supernatant. Expi293F cells were transfected using Expifectamine (Gibco, A14524) following the manufacturer's recommended protocol. Antibody presence in supernatant was detected using Coomassie blue stained SDS-PAGE gels and western blots. Supernatant was harvested for purification via Ni-NTA agarose (QIAGEN 30210) or HA agarose affinity columns (Sigma-Aldrich, A2095-1 ml) depending on the respective tag in the construct. Protein concentrations were confirmed using BCA assays (Pierce, 23235) and constructs were buffer exchanged using Amicon spin columns (Millipore, UFC501096) before adding protein stabilizer (2% V/V) used in commercial blinatumomab formulations (Amgen, Thousand Oaks).

FIG. 2 provides a graphical representation of the BiTE constructs. For construction of αCD22xαCD3 BiTEs, the overall framework of blinatumomab was emulated. Blina's relatively simple architecture. As shown in FIG. 2, blinatumomab consists of the two scFVs (for CD3 and CD19), centrally connected by a short flexible linker (GGGGS). The variable heavy and light chains of the two scFv moieties are arranged in the pattern vLvH-linker-vHvL, with flexible glycine-serine linkers between the heavy and light chains. The alternating heavy/light chain pattern is thought to be important to avoid intermolecular mispairing of opposing heavy and light chain fragments. Loffler et Al. (2000) A recombinant bi-specific single-chain antibody CD19XCD3, induces rapid and high lymphoma-directed cytotoxicity by unstimulated T lymphocytes Blood 95:6. Notably, the absence of any crystallizable fragment (Fc) binding moiety in the BiTE, while allowing for simplicity of construction, is known to severely limit the half-life of the protein in vivo. Therefore, in addition to mimicking blina's architecture, various central linker modifications that may enhance pharmacokinetics and stability in vivo or provide an additional attachment moiety (handle) to create tri-functional molecules were explored. However, these studies focused on BiTE 197 (SEQ ID NO: 31 with a 6x His terminal tag) (which uses a central SGGCGGS linker). BiTE 197 was pursued based on in vitro cytoxicity and T cell activation at doses and levels comparable to blina.

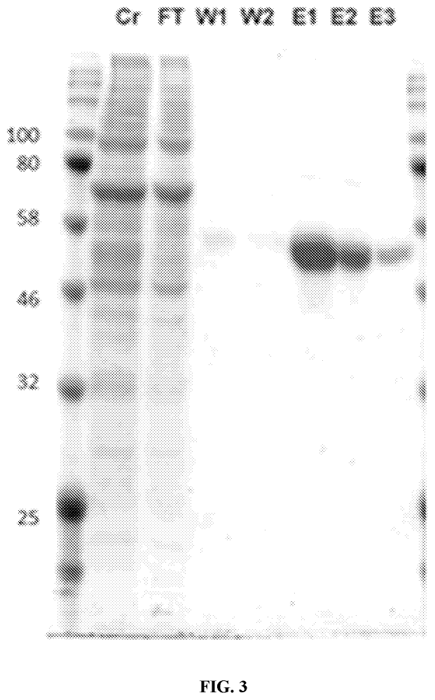

FIG. 3 provides a SDS-PAGE gel with Coomassie blue staining of the crude and fractions from Ni-NTA purification of BiTE-197. Samples were run under reduced, denaturing conditions. Lanes are labeled as follow: Cr=crude culture supernatant; FT=Flow through (unbound) fraction; W=wash fractions; E=elution fractions. Relative molecular weights (in kDa) are indicated on the left. Predicted molecular weight=53.9 kDa.

Sufficient quantities of the BiTE were produced for further studies by purifying the antibody directly from mammalian cell culture (Expi293F) supernatant and purifying with a 6xHis-tag affinity column. Binding of each individual arm, in the context of the BiTE was verified to its cognate target as discussed in Example 2 below.

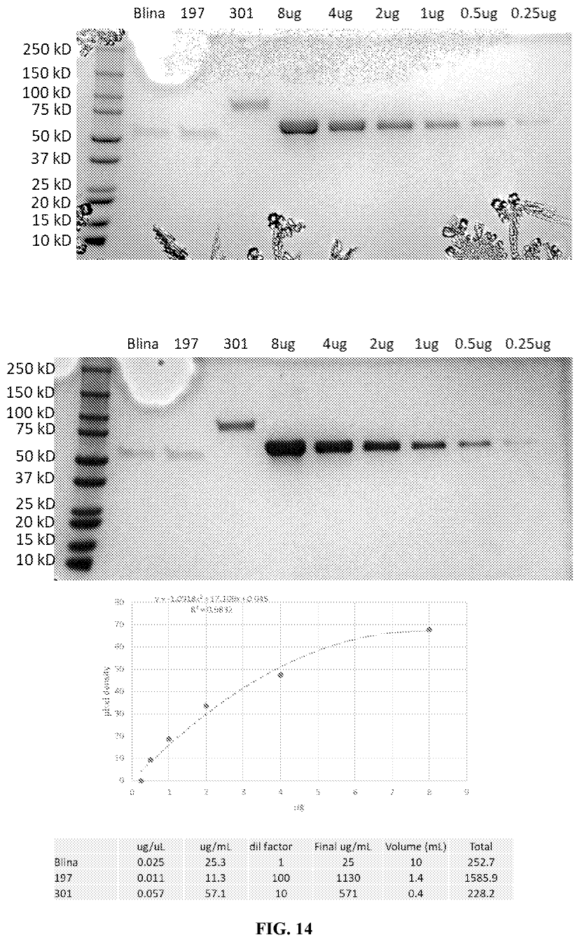

Tri-functional engager 301 was prepared via a similar method and the corresponding SDS-PAGE gel with Coomassie blue staining is shown in FIG. 14.

### Example 2: Binding Characterization of Constructs by Flow Cytometry

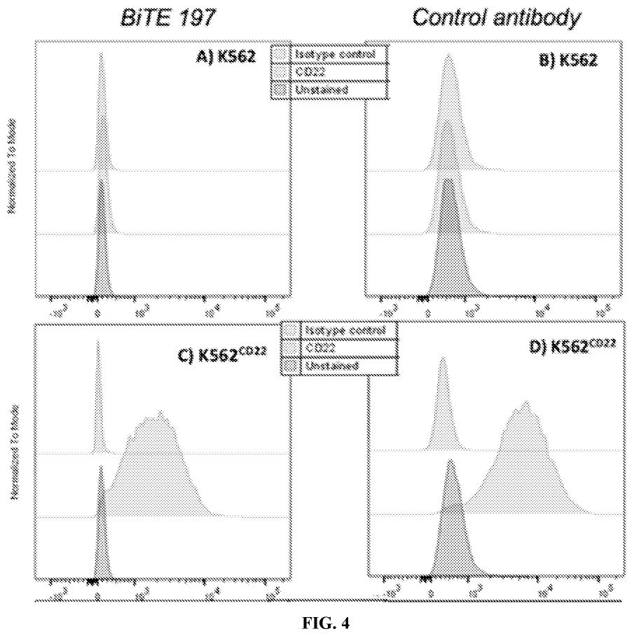

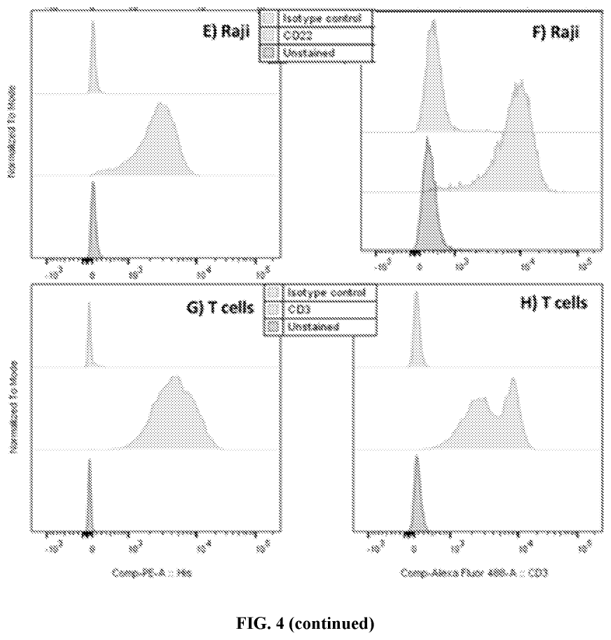

A FACS Fortessa 2000 flow cytometer was used to characterize binding of αCD22xCD3 constructs to CD22 and CD3 targets. Non-Hodgkin's Lymphoma (NHL) cell line Raji (CD22-positive control), T cells (CD3-positive control) isolated from donor peripheral blood mononuclear cells (see below for methods) and K562 cells transduced with a CD22 expression cassette were used as controls. Cells were washed with PBS and incubated with each construct for 30 minutes, then washed again with PBS before 30-minute incubation with appropriate secondary antibody. ZNIR was used to identify live cells. Binding was reported in singlet, ZNIR-cell populations. FIG. 4 provides the flow cytometry data for different cell types.

To test binding of BiTE 197 to its CD22 cognate target, cells were incubated with BiTE 197 at 1 ug/mL and then probed with a secondary anti-His-PE before flow cytometry analysis. K562 cells do not natively express CD22 as confirmed with a research grade αCD22-BV421 antibody (B) and BiTE 197 (A). K562 cells were tranduced to express CD22 (D) and binding of BiTE 197 was confirmed (C). Raji cells natively express surface CD22 in high quantity and BiTE 197 demonstrated binding to Raji cells (E) as did a control αCD22-BV421 antibody (F). Finally, T cells isolated from healthy PBMCs were incubated with either BiTE 197 then αHis-PE (G) or αCD3-AlexaFluor 488 (H) to confirm binding to CD3 target.

### Example 3: Cell Killing Assays

5×10⁶ Granta non-Hodgkin's lymphoma cells in 1 mL 5% heat-inactivated fetal bovine serum in PBS (HI-FBS/PBS) were stained with 1 μL CFSE (eBioscience) for 5 minutes at room temperature. Labeling was stopped by adding 2-3 mL HI-FBS/PBS and culture medium to 15 mL. Cells were centrifuged (5 minutes, 300×g, room temperature), resuspended in culture medium and allowed to recover for 2-16 hours. 2×10⁴ CFSE-Granta cells were incubated with PBMCs at different effector:target (E:T) ratios in 96-well round-bottom plates, in a final volume of 200 μL containing 500 IU/mL rhIL-2. Treatment consisted of PBS (negative control), BiTE #197, blinatumomab or BiTE #198 (αCD22-ABD-αCD3) at 0.1, 1 or 10 μg/mL.

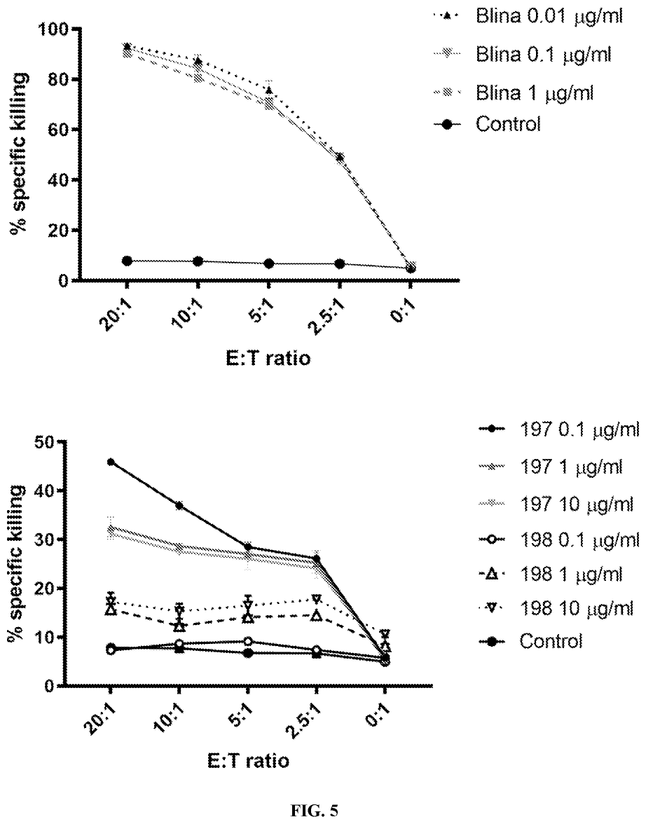

After 12-16 hours, cells were centrifuged, washed with PBS twice and resuspended in 100 μL ZNIR (1:1000 dilution in PBS) for 30 minutes at 4° C. Cells were washed with 2% HI-FBS/PBS, fixed with 4% paraformaldehyde in PBS, washed again with 2% HI-FBS/PBS and resuspended in the same buffer for acquisition in a Fortessa (BD) flow cytometer. Flow data was analyzed with FlowJo. Single cells were gated on SSC-H/SSC-W. The percentage of specific killing was defined as the percentage of ZNIR+ events within the single cell/CFSE+ gate. Data are shown in FIG. 5.

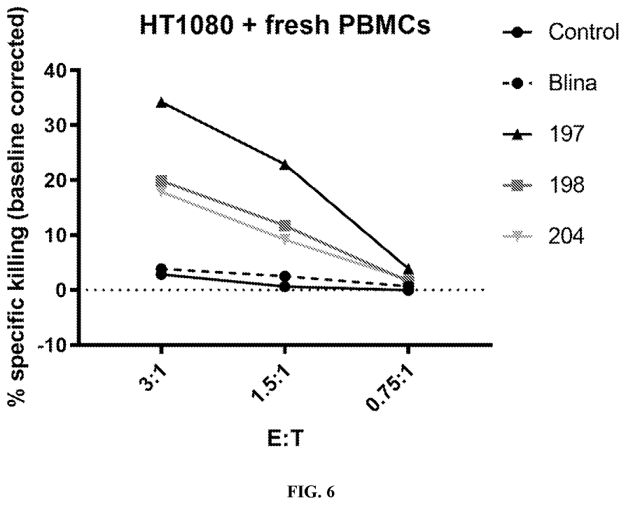

In another set of experiments, HT1080 sarcoma cells were labeled with CFSE as above and used as target cells in killing assays as described above. Unstimulated PBMCs were used as effector cells. Treatment consisted of BiTE #197 and BiTE #198 (αCD22-ABD-αCD3). Data are shown in FIG. 6.

ABD refers to an albumin binding domain. The CD22 and CD3 binding domains were the same for all constructs. αCD22-ABD-αCD3 was structured as αCD22-(GGGGS)₃-ABD-(GGGGS)₃-αCD3-6xHis. BiTE #197 has a structure of αCD22-SGGCGGS-αCD3-6xHis where HA is a hemagglutinin tag.

### Example 4: T-Cell Activation with αCD22xαCD3 BiTE

Trima Leukocyte Reduction Chambers from healthy donors were purchased from Vitalant. Peripheral blood mononuclear cells (PBMCs) were isolated from the buffy coat of whole blood using Ficoll-Paque Premium (GE Healthcare Biosciences). PBMCs were incubated in RPMI media prepared as described above at 37° and 5% CO2, CD3+ T-cells were isolated from PBMCs using MACS Pan T cell isolation kit (Miltenyi, 130-096-535) following the manufacturer's protocol.

T cells were isolated from PBMCs that had been thawed and rested overnight in cytokine-free media. The T cells were incubated with CFSE labeled NALM-6 or U2973 target cells and treated with BiTE 197, blina, or 197+blina. After 24 hours, live T cells (distinguished by ZNIR staining), were analyzed via flow cytometry for activation and proliferation markers CD25 and CD69.

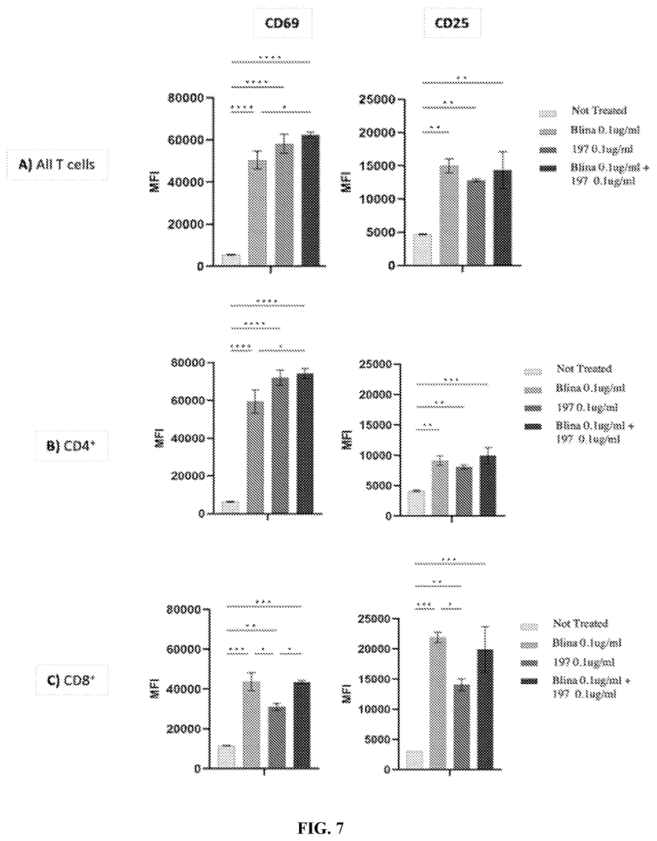

To assess the effects of BiTE molecules on T-cell activation, a panel of T cell phenotypic markers was profiled upon exposure to BiTEs 197, blina or 197+blina in the presence of target cells after 24 hours. The panel included the T-cell activation and proliferation markers CD25 and CD69, which are used extensively to quantify T cell activation [Liu L, Lam C K, Long V, Widjaja L, Yang Y, Li H, Jin L, Burke S, Gorlatov S, et. Al (2017) MGD011, A CD19 x CD3 Dual-Affinity Retargeting Bi-specific Molecule Incorporating Extended Circulating Half-life for the treatment of B-Cell Malignancies. Clin Cancer Res. 23(6):1506-1518; Dreier T, Baeuerle P A, Fichtner Iduna, Grün M, Schlereth B, Lorenczewski G, et Al. (2003) T Cell Costimulus-Independent and Very Efficacious Inhibition of Tumor Growth in Mice Bearing Subcutaneous or Leukemic Human B Cell Lymphoma Xenografts by a CD19−/CD3− Bispecific Single-Chain Antibody Construct. J Immunol; 170:4397-4402; Rossi D L, Rossi E A, Cardillo T M, Goldenberg D M, Chang C H. (2014) A new class of bispecific antibodies to redirect T cells for cancer immunotherapy. mAbs, 6:2, 381-391, doi: 10.4161/mabs.27385]. T-cells were incubated with target tumor cells, with or without BiTE constructs. Significantly increased BiTE-dependent T-cell activation was observed after 24 hours. All treatments increased expression of CD25 and CD69 (FIG. 7). It was notable that T cells treated with BiTE 197+blina exhibited statistically higher CD69 levels when compared to blina alone, with the CD4+ population exhibiting the highest relative increase. Taken in total, BiTE 197 was comparable to blina in the induction of a rapid and increased T cell activation as evidenced by increase of CD25+ and CD69+ T cells. This, in addition to the favorable cytotoxicity levels in the dual-treated samples, provided further rationalization for an in vivo assessment of BITE 197.

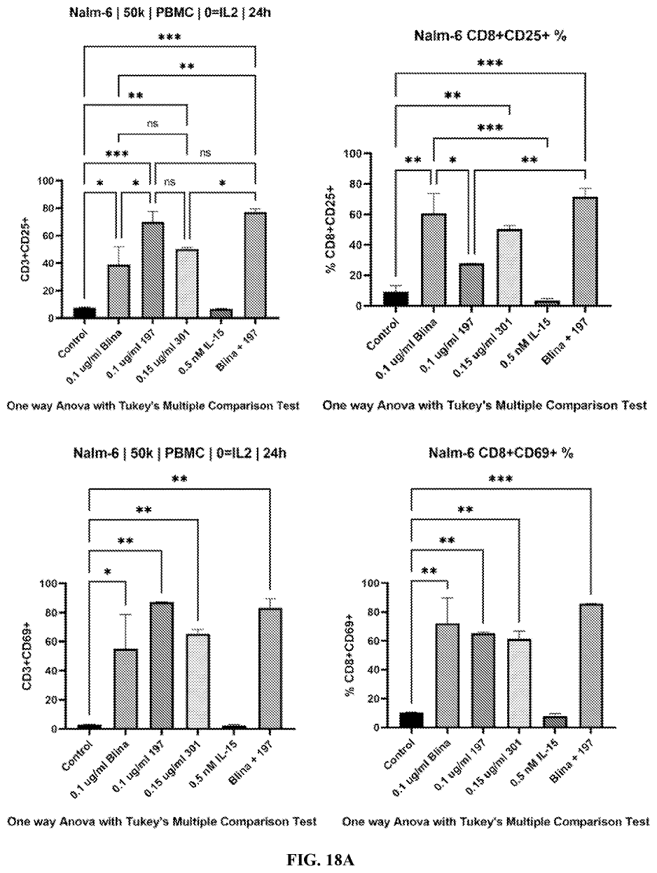

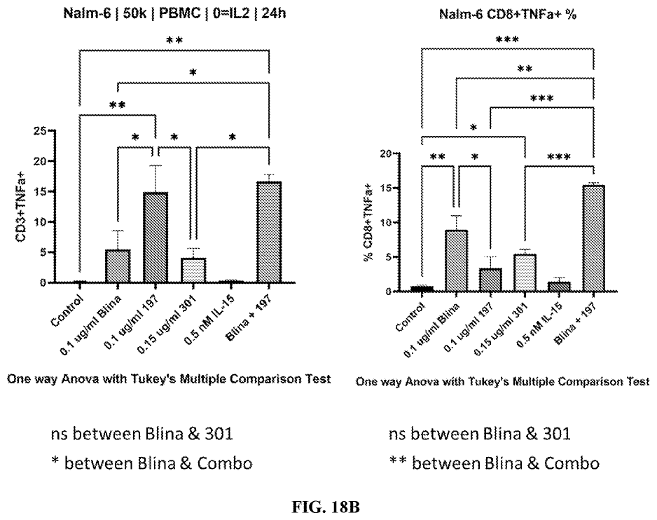

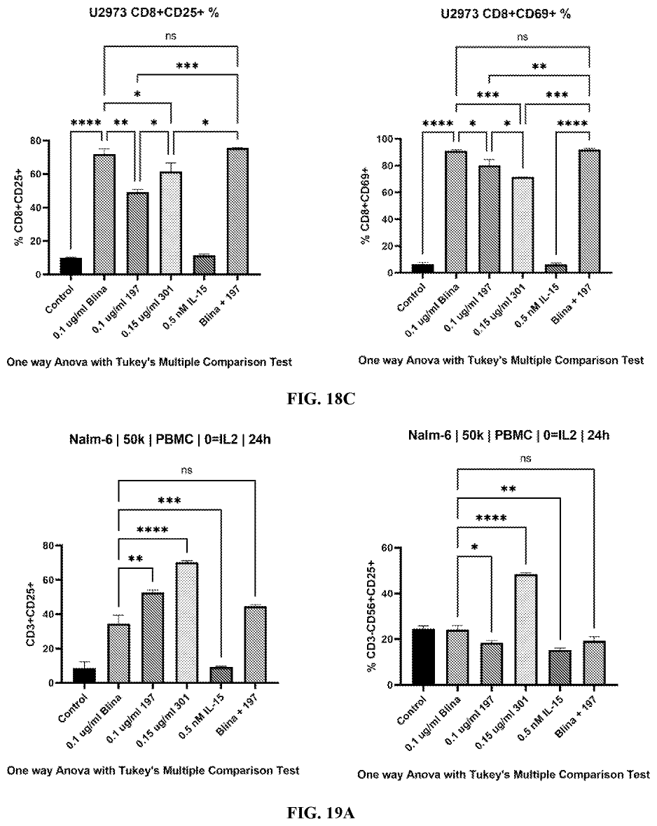

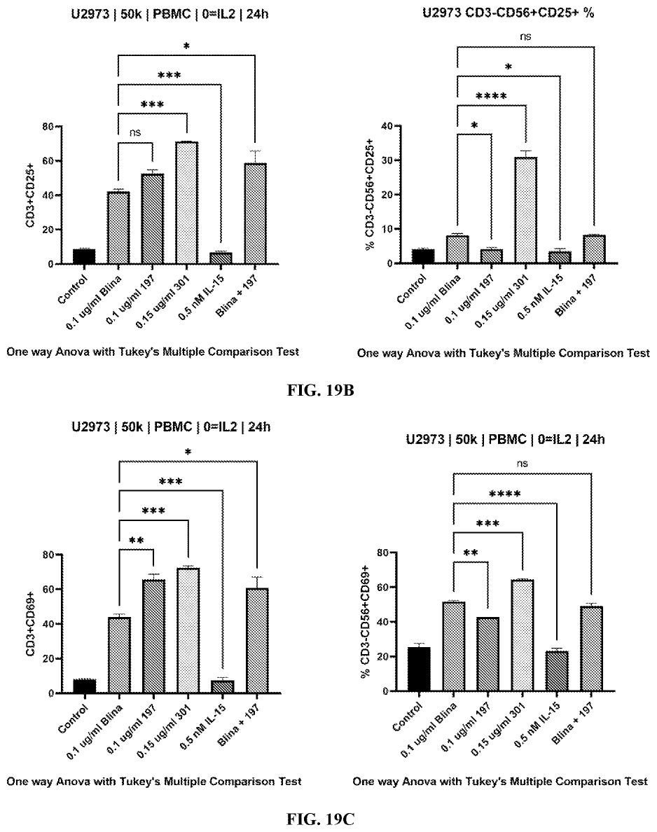

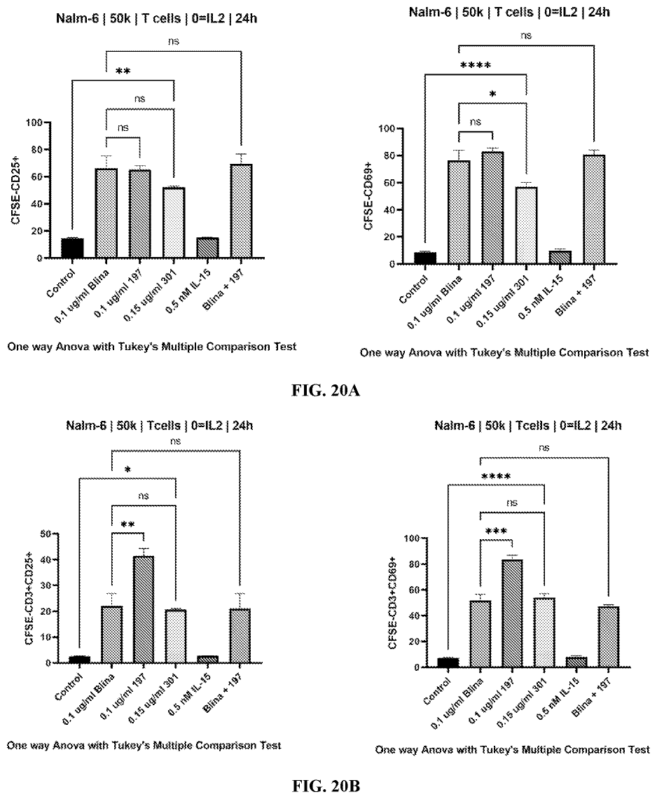

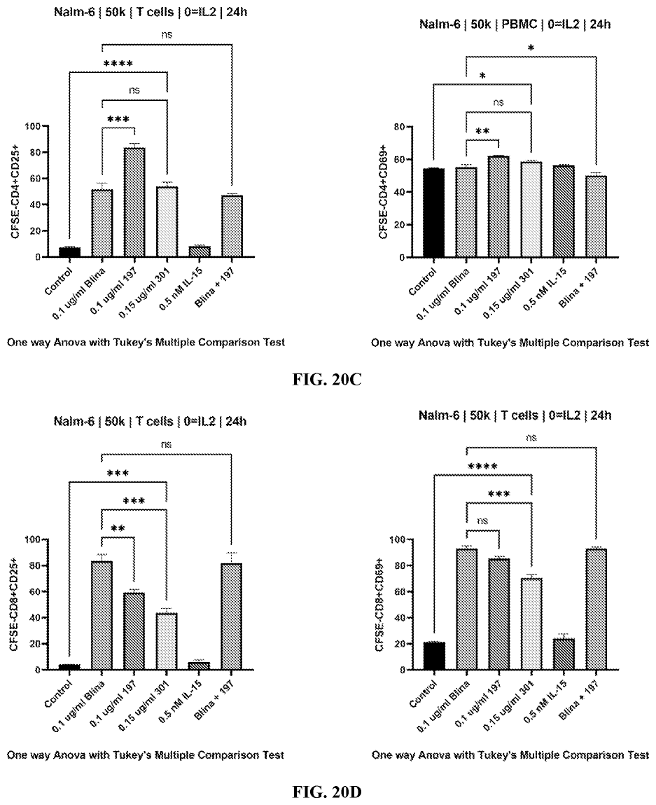

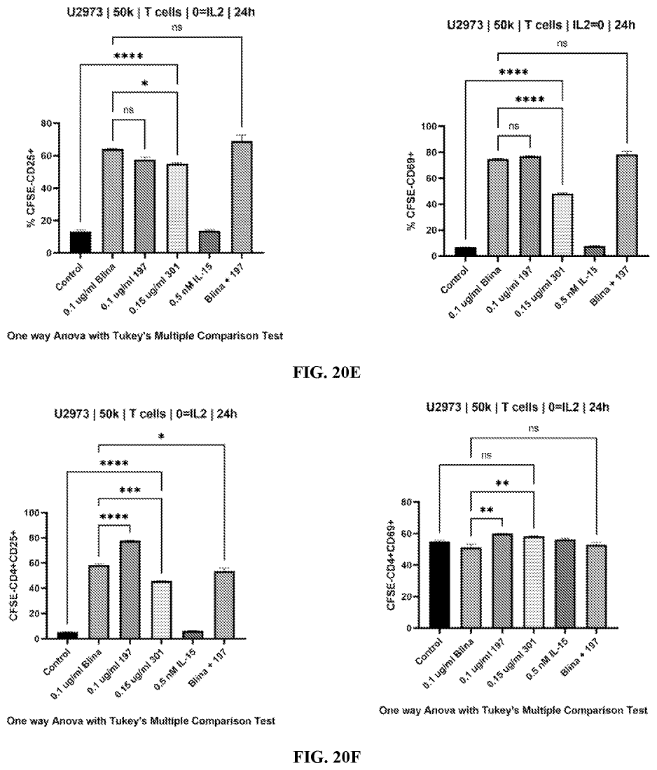

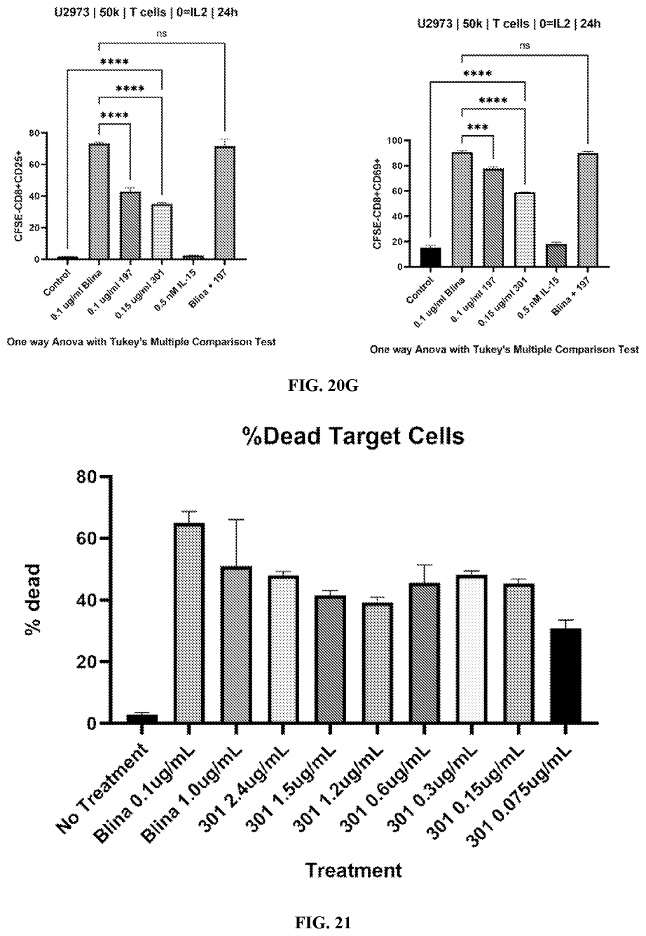

The same study was also performed with Blina, BiTE 197, tri-functional engager 301 and IL-15 with the results provided in FIGS. 18A-18C, 19A-19C and 20A-20G. For FIGS. 18A-18C, the assays were performed as described in FIG. 7, and activation results were presented for T-cells present in the PBMC population (i.e. CD3+ cells encompassing all T cells, or the CD8+ or CD4+ subpopulations of T cells). The different treatments of interest (Blina, 197, the tri-functional antibody and the Blina-197 combination) alternatingly elicited the highest level of T-cell activation. Nevertheless, both 197 and the tri-functional antibody always elevated activation significantly compared to control. Importantly, treatment with the tri-functional antibody, compared with equimolar amounts of recombinant IL-15, suggested that the activation effect of the trifunctional molecule was due to its targeting capability, and was not mediated through the non-specific addition of IL-15 to the culture. With respect to FIGS. 19A-19C, these data demonstrate that in addition to elevation of the CD25 and CD69 in the T-cell compartment, we also observed activation in the Natural Killer (NK) (CD3-CD56+) cell compartment. Interestingly, the NK activation was most prominently seen in conjunction with the tri-functional antibody treatment. This may be expected, as the IL15-Sushi moiety present in the molecule can activate NK cells, as well as T cells. In fact, one potential benefit of this therapeutic molecule is that in addition to activating T-cells, it may also enhance NK activity at the site of the tumor, thereby increasing the overall immunostimulatory effect and efficacy of the drug. Experiments were performed as described in FIG. 7. With respect to FIGS. 20A-20G, activation markers CD25 and CD69 were examined among subsets of different populations of T-cells, and significant increases in activation mediated by all the antibody treatments (Blina, 197, the tri-functional antibody, and the Blina-197 combination) were observed. Experiments were performed as described for FIG. 7.

### Example 5: Cytotoxicity Against ALL/NHL Cell Lines

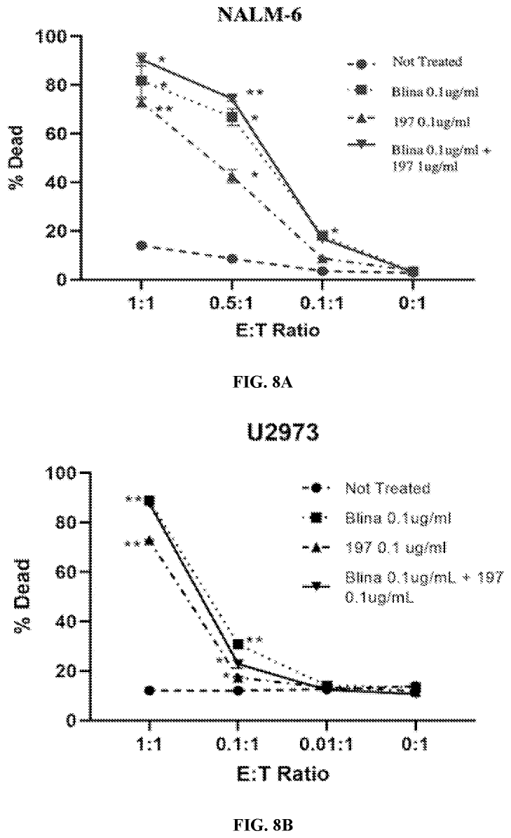

CD22 is expressed broadly on B-cell lymphomas and leukemias and is therefore a suitable alternative target to CD19. Notably, however, CD22 and CD19 display widely different behaviors when it comes to their internalization upon binding (CD22>>CD19) and the cell-surface density on malignant B-cells (CD19>>CD22) [Du X, Beers R, Fitzgerald D J, Pastan I. (2008) Differential cellular internalization of anti-CD19 and -CD22 immunotoxins results in different cytotoxic activity. Cancer Res 68 (15): 6300-6305]. These innate differences in the two surface molecules must be considered when comparing of CD19- and CD22-targeted constructs. Nevertheless, αCD22xαCD3 BiTE demonstrated effectiveness in PBMC-mediated killing of the ALL-cell line NALM-6 and the double hit NHL cell line U2973 at levels comparable to blinatumomab (FIGS. 8A-8B). Both blina and BiTE 197 demonstrated cytotoxicity greater than 70% against NALM-6 cells at an ET ratio of 1:1 (FIG. 8A). Additionally, both blina and BiTE 197 killed more than 70% of U2973 cells (FIG. 8B), also at a 1:1 E:T. While several CD22 BiTE constructs were tested (data not shown), BiTE 197 demonstrated superior cytotoxicity in vitro, perhaps due to its short linker length, and therefore that reagent was the focus for further studies.

NALM-6 or U2973 cells were CFSE labeled following manufacturer's protocol and co-cultured as target cells with PBMCs and BiTE constructs to determine enhanced tumor cell killing. PBMCs were rested overnight (16 hours) in cytokine-free media prior to initiating killing assays. 50,000 target cells were seeded into 96-well U-bottom plates (Falcon 08-772-3B). PBMCs were added to target cells at various E:T ratios. Single or combined treatments of αCD22xαCD3 BiTE constructs and blinatumomab were added at the indicated concentrations and incubated with PBMC effectors for 24 h. Untreated control groups contained PBMCs but no BiTE treatment. IL-2 was included in some experiments, as indicated, at the designated concentrations. Target cell killing was determined using flow cytometry and analyzed using FlowJoTM v10.8.1 software. Single cell events were determined with side and forward scattering to gate out cell fragments. Live target cells were gated using CFSE live cell staining. Dead cells were gated using Zombie Near Infrared (ZNIR) staining. Target cell killing was defined by the percentage of CFSE+ single target cells that stained positive for ZNIR.

Additionally, the effects of combination treatment with blinatumomab and BiTE 197 were tested. At low E:T ratios of 1:1 and 0.5:1, blina killed 82% and 66% of target NALM-6 tumor cells while BiTE 197 killed 73% and 42% (FIG. 8A). However, the combination of blina and 197 killed 90% and 74% of target cells at the same respective E:T ratios. While not statistically significant, the combination of blina and 197 demonstrated a trend that suggested superiority over blina alone. At the lowest E:T ratio of 0.1:1, the combination treatment killed twice as much as blina (16% vs 8%). These results suggest that the combination is potentially more effective, thus providing further impetus to explore the combinatory effect in vivo.

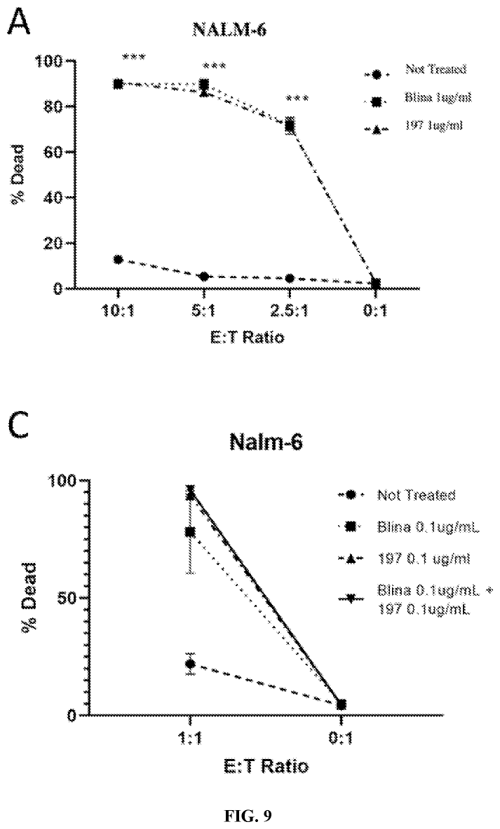

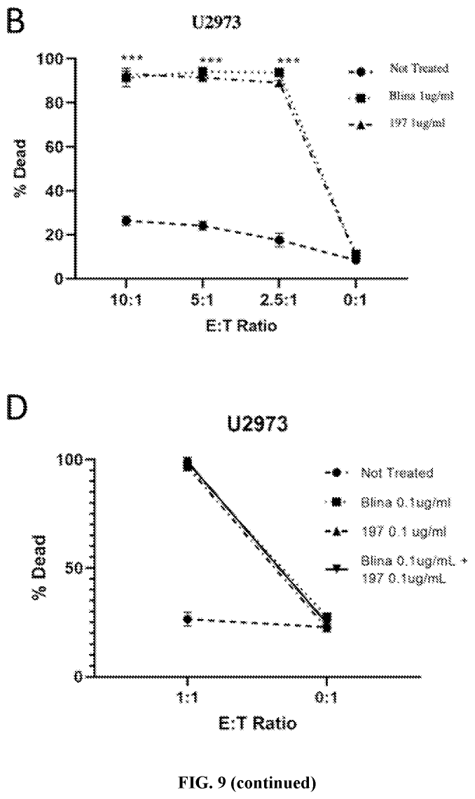

Finally, as part of our cytoxicity studies, experiments were performed at higher E:Ts, varied antibody concentrations (up to 1 ug/mL for both blina and BiTE 197), as well as with added exogenous IL-2. As shown in FIG. 9, the results suggested that in the absence of exogenous IL-2 (as would be the case in vivo), blina efficacy reached an in vitro plateau at 0.1 ug/mL, while BiTE 197 displayed maximal in vitro activity (essentially matching blina) at 1 ug/mL.

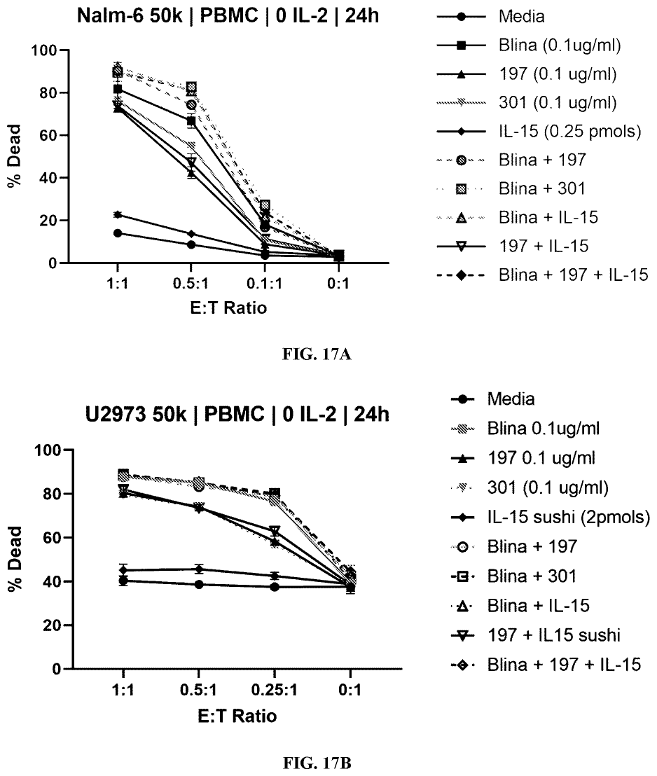

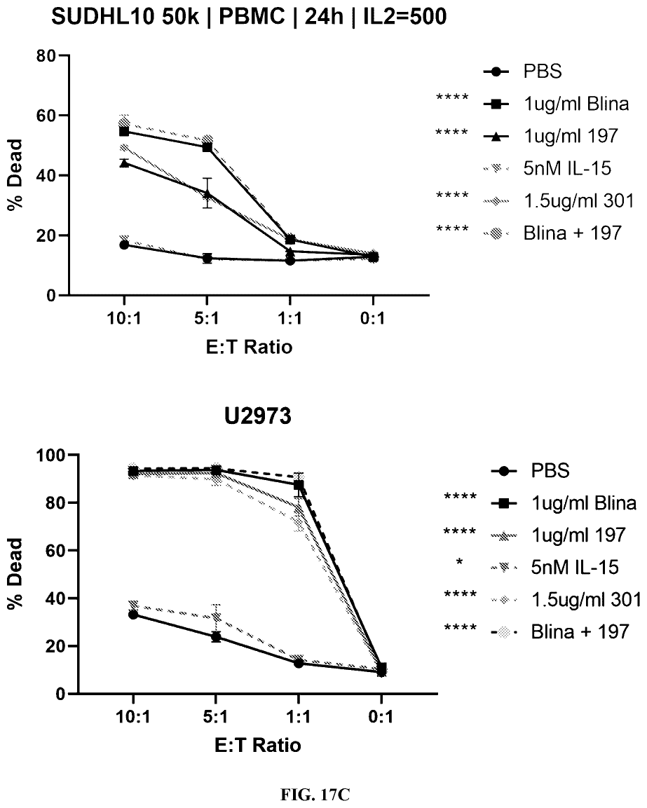

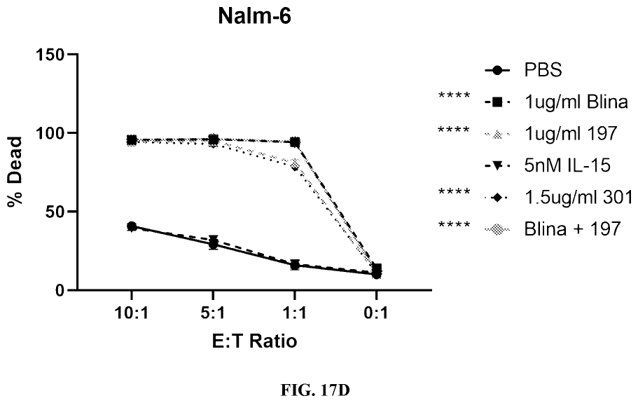

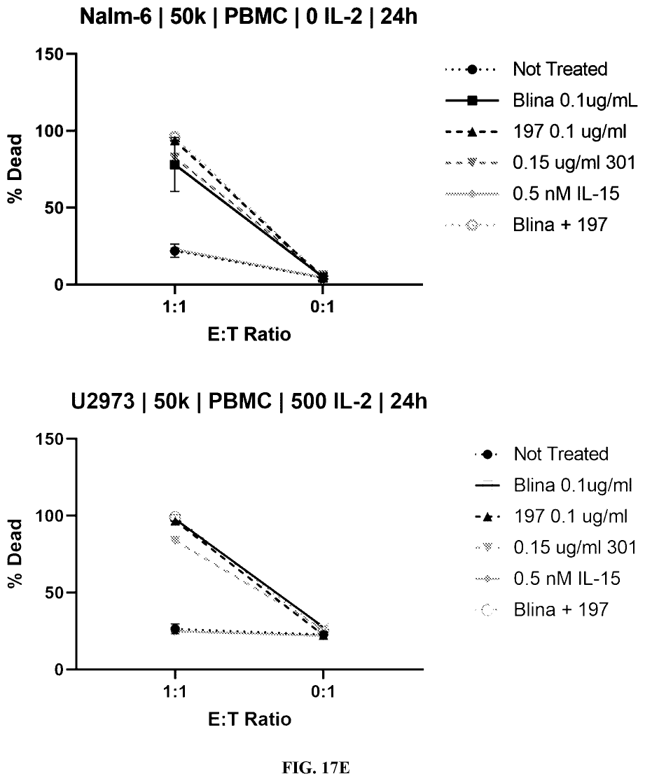

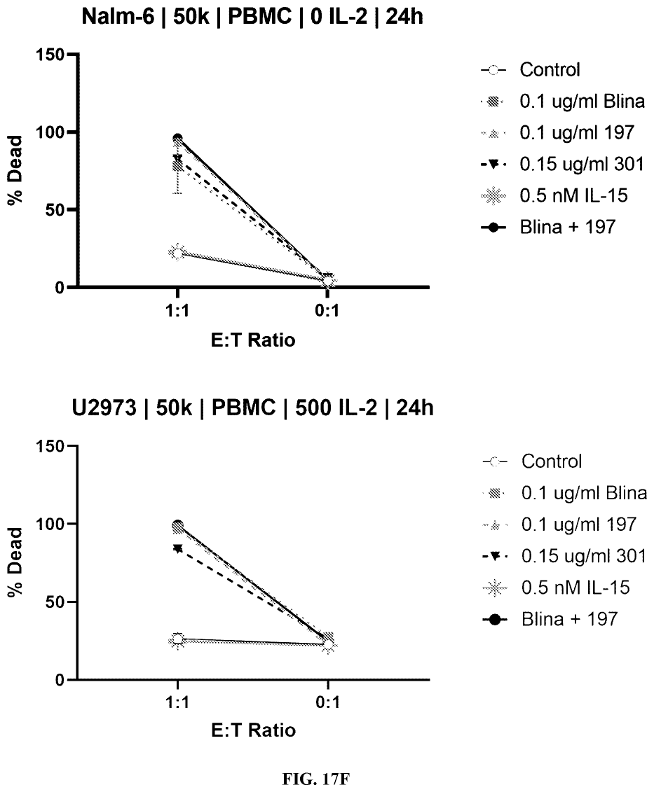

Another study was conducted under the same conditions but including tri-functional engager 301, with the results shown in FIGS. 17A-17F and 21. Note that in the figures, the tri-functional antibody is varying referred to as “301.” The inclusion of IL15 only or IL15-sushi treatments was to establish that cytotoxicity was not due solely to the T-cell stimulation provided by IL-15 or the IL-15-Sushi superagonist. While Blina typically induced the best in vitro cytotoxicity results, both 197 and the tri-functional antibody were quite often nearly equivalent to the clinical-grade Blina in cytolytic efficiency across the different cell lines and test conditions. With respect to FIG. 21, the effector cells (PBMCs) were co-incubated with the target CFSE-labeled Nalm-6 cells at an ET ratio of 1:1 overnight for 16 hours. The indicated treatments were applied, and toxicity was determined via a flow cytometry based readout. While the tri-functional antibody had slightly lower cytotoxic activity than the clinical-grade Blina at the concentrations tested, it is interesting to note that the our treatment molecule showed no loss of activity over a >2-log reduction in concentration from 2.4 ug/mL to 0.15 ug/mL.

### Example 6: In Vivo Studies

The ALL cell line NALM-6 was selected for in vivo murine xenograft studies based on our in vitro cytotoxicity studies as well as previously published work with blina [Dreier T, Baeuerle P A, Fichtner Iduna, Grün M, Schlereth B, Lorenczewski G, et Al. (2003) T Cell Costimulus-Independent and Very Efficacious Inhibition of Tumor Growth in Mice Bearing Subcutaneous or Leukemic Human B Cell Lymphoma Xenografts by a CD19−/CD3− Bispecific Single-Chain Antibody Construct. J Immunol; 170:4397-4402]. A blina dose of 0.1 ug/mouse was chosen, following the protocol established in [Dreier T, Baeuerle P A, Fichtner Iduna, Grün M, Schlereth B, Lorenczewski G, et Al. (2003) T Cell Costimulus-Independent and Very Efficacious Inhibition of Tumor Growth in Mice Bearing Subcutaneous or Leukemic Human B Cell Lymphoma Xenografts by a CD19−/CD3− Bispecific Single-Chain Antibody Construct. J Immunol; 170:4397-4402], a study which also demonstrated comparable efficacy of 1 ug and 0.1 ug/mouse dosages. BiTE 197 was administered at a higher dose of 1 ug/mouse because of a) the known greater propensity of CD22 to internalize upon antibody binding [Du X, Beers R, Fitzgerald D J, Pastan I. (2008) Differential cellular internalization of anti-CD19 and -CD22 immunotoxins results in different cytotoxic activity. Cancer Res 68(15): 6300-6305] (up to 10-fold greater than CD19); b) the established 5-to-6 fold lower surface expression of CD22 compared to CD19 on NALM-6 cells [Haso W, Lee D W, Shah N N, Stetler-Stevenson M, Yuan C M, Pastan I H et al. (2013) Anti-CD22-chimeric antigen receptors targeting B-cell precursor acute lymphoblastic leukemia. Blood. 121(7):1165-74]; and C) the assumed greater purity and stability of a commercial product (blina) versus our lab-synthesized construct. Based on these combined differences, a BiTE 197 dosage of 1 ug/mouse was selected for in vivo studies.

All work with animals was performed in accordance with the national and international guidelines under protocols approved by the University of California Davis Institutional Animal Care and Use Committee (AAALAC accreditation #000029; PHS Animal Assurance #A3433-01; USDA Registration #93-R-0433).

A schematic of the test plan is shown in FIG. 10. NOD scid gamma (NSG) mice were obtained from Jackson Laboratories and kept at UCDMC vivarium in accordance with Institutional Animal Care and Use Committee (LACUC) protocols until 6 weeks old. All mice were inoculated simultaneously with 10⁴ NALM-6 cells and 10⁷ isolated unstimulated PBMCs combined into a volume of 100 uL via subcutaneous (S.C.) injection resulting in an E:T ratio of 1000:1, as previously described [Dreier T, Baeuerle P A, Fichtner Iduna, Grün M, Schlereth B, Lorenczewski G, et Al. (2003) T Cell Costimulus-Independent and Very Efficacious Inhibition of Tumor Growth in Mice Bearing Subcutaneous or Leukemic Human B Cell Lymphoma Xenografts by a CD19−/CD3− Bispecific Single-Chain Antibody Construct. J Immunol; 170:4397-4402]. After 1 hour, mice were treated intravenously (I.V.) with either blinatumomab (0.1 ug/mouse), BiTE 197 (1 ug/mouse), or PBS in 100 uL aliquots. Mice received 5 total treatments of the same dose on days 0, 1, 2, 3, 4. Blood was collected and pooled from saphenous vein bleeds from two mice per group weekly for 3 weeks for assessment of hematologic, hepatic and renal toxicity. Blood work analysis was performed by the Comparative Pathology lab core at UC Davis Veterinarian Medicine at baseline, day 8 and day 15 after first treatment. Mice were weighed twice weekly and sacrificed upon greater than 15% weight loss from start of treatment or when hind limb paralysis occurred.

This same study was repeated, except in study 2, NALM-6 cells were delivered as xenograft I. V. through the tail vein. In this study, the synergistic effect of BiTE combination was assessed with the addition of a group treated with both blinatumomab (0.1 ug/mouse) and BiTE 197 (1 ug/mouse). Control group (PBS), blinatumomab, and BiTE 197 groups were repeated as described above.

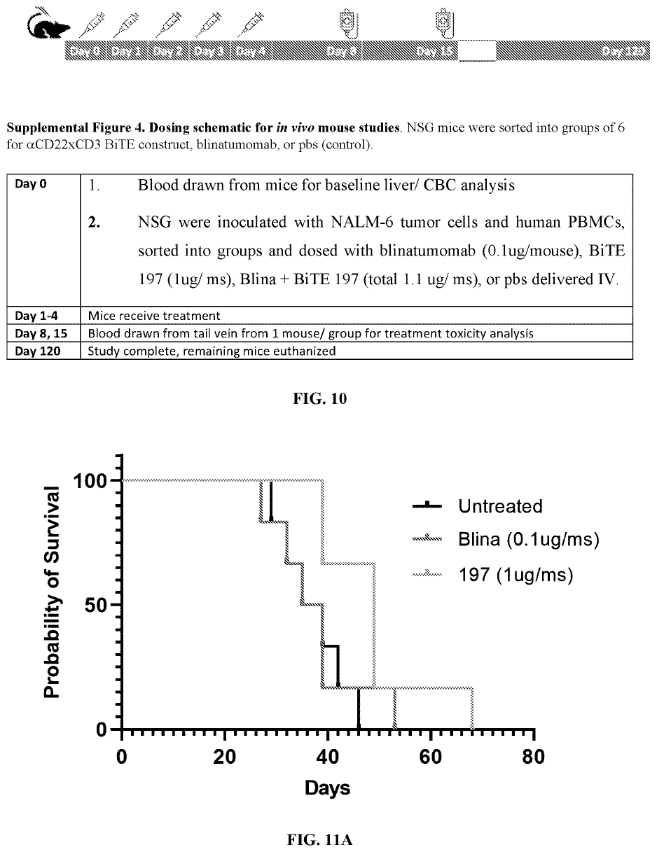

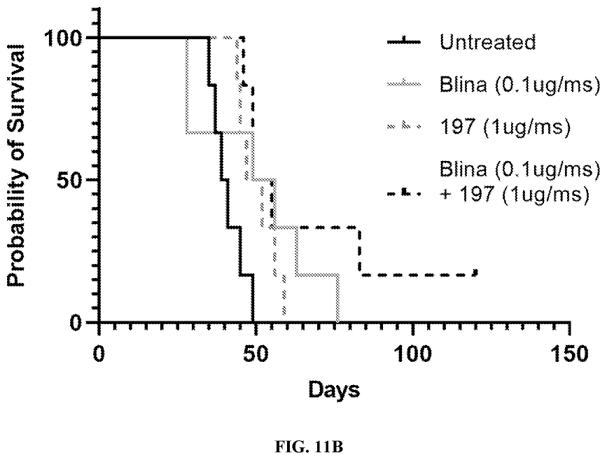

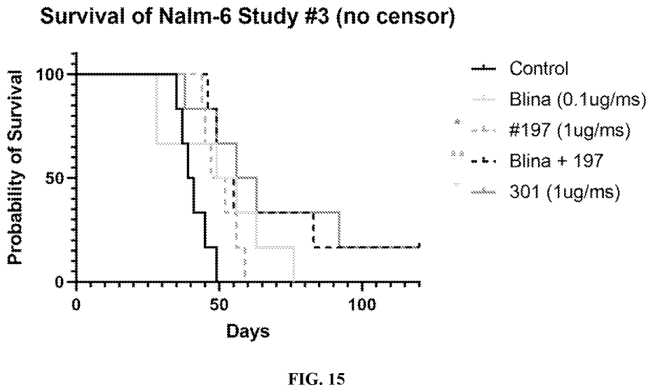

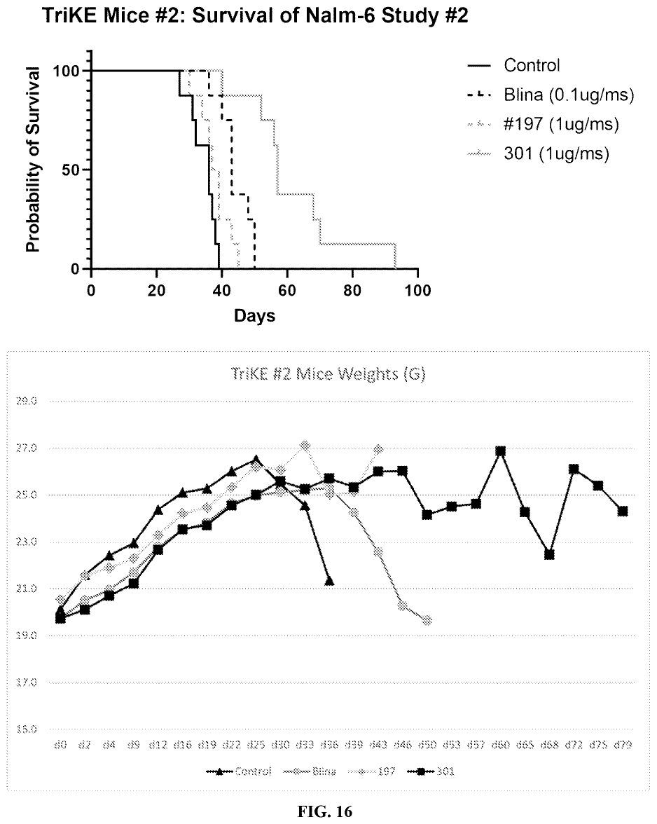

Survival data from studies 1 and 2 are reported in table 5. In study 1, a subcutaneous leukemia model was used and 4/6 mice treated with BiTE 197 survived longer than 50 days while only 2/6 mice treated with blina reached the same milestone (FIG. 11A). To validate the results of study 1 and assess the efficacy of combination therapy, a similar model was established with I.V. injection of tumor cells. In study 2, all treatment groups-blina, BiTE 197, and combination blina+197—performed similarly with 3/6 mice surviving past day 50 (FIG. 11B) in each group. An additional treatment arm using tri-functional engager 301 at 1 μg/mouse was also included and survival data with this treatment arm are shown in FIG. 15, data on mouse weights and survival from a study conducted under the same conditions are provided in FIG. 16. However, 2/6 mice in the combination treatment group survived longer than the maximal survival time for either blina or BiTE 197 alone (>76 days), suggesting a possible treatment benefit from concurrent dosing. Further studies are planned to elucidate the mechanism for this potential synergy and confirm its efficacy. All untreated control mice survived less than 50 days in both studies.

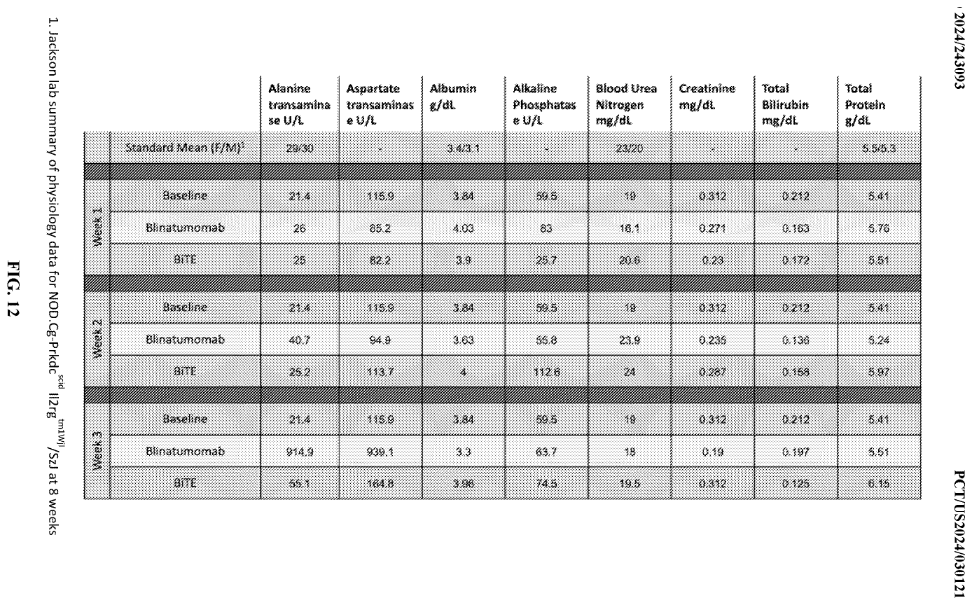

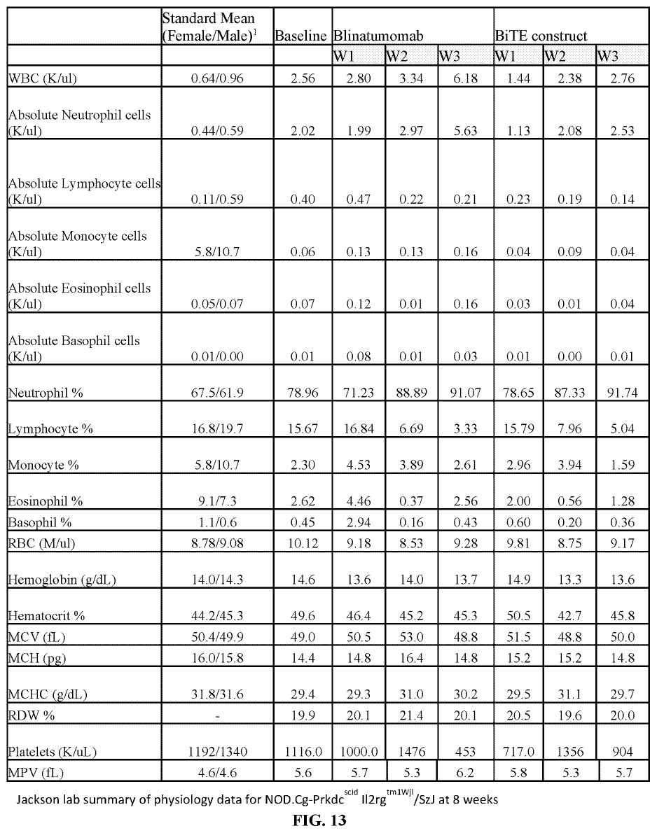

Treatment toxicity was assessed in study 1. Hepatic and hematologic toxicity data is presented in FIGS. 12-13. No significant treatment-related toxicities were observed with treatment using either 197 or blina. Elevated AST and ALT were observed in the blina treated mice during the 3^{rd} week.

FIG. 12 provides a blood panel to detect treatment mediated toxicities. Baseline value was taken before xenograft transplantation and is listed in each time period for reference only. Mice treated with either blinatumomab or BiTE were examined weekly post treatment for the first 3 weeks. Each reading represents sample from one mouse selected at random at each time interval within appropriate treatment group.

FIG. 13 provides full CBC for treated NALM-6 xenograft mice.

TABLE 5
Survival of NALM-6 Xenograft NSG Mice
A)

| | Population | Median |
|---|---|---|
| Group | size | Survival |
| Control (pbs) | N = 6 | 37 days (29- |
| | | 46) |
| Blinatumomab (0.1 ug/ | N = 6 | 37 days (27- |
| mouse) | | 53) |
| BiTE 197 (1 ug/mouse) | N = 6 | 49 days (39- |
| | | 68) |

B)

| | Population | Median |
|---|---|---|
| Group | size | Survival |
| Control (pbs) | N = 6 | 40 days (35- |
| | | 49) |
| Blinatumomab (0.1 ug/ | N = 6 | 52.5 days (28- |
| mouse) | | 76) |
| BiTE 197 (1 ug/mouse) | N = 6 | 49.5 days (44- |
| | | 59) |
| Blinatumomab (0.1 ug/ | N = 6 | 52 days (46- |
| mouse) + 197 (lug/mouse) | | 120) |

*Survival statistics from in vivo A) Study 1 (p = 0.0245); B) Study 2 (p = 0.0151). Long rank (Mantel Cox) test was used for group difference analysis.

In this study, successful production and characterization of a novel αCD22xαCD3 BiTE using an architecture analogous to the therapeutic agent blinatumomab was demonstrated. Multiple constructs, containing the same binding domains with varying linkers, were developed, but only BiTE 197 is presented here based on its superior in vitro efficacy that compares favorably to blina.

Given its structural similarity to blina, BiTE 197, when used as a single agent, would be expected to display many of the same pharmacokinetics as the FDA-approved drug. These include a short in vivo half-life that would likely require continuous IV infusion and a similar dosing schedule. While second- and third-generation BisAbs have demonstrated prolonged retention and more convenient dosing, this format was retained to facilitate comparison with blina. As noted, a direct equimolar comparison of dosing between blina and 197 is challenging due to a number of factors, including differential internalization behavior and antigen density of CD19 and CD22 on target tumor cells. In regards to therapeutic usage, as CD19- and CD22-based CAR-T therapies have shown similar side effects in terms of cytokine release syndrome and neurotoxicity [Du X, Beers R, Fitzgerald D J, Pastan I. (2008) Differential cellular internalization of anti-CD19 and -CD22 immunotoxins results in different cytotoxic activity. Cancer Res 68(15): 6300-6305] it is possible that single-agent therapy with BiTE 197 could present the same spectrum of manageable toxicities as CD19 BiTE.

In clinical studies, blinatumomab typically produces a high initial response rate [Goebeler M E, Knop S, Viardot A, Kufer P, Topp M S, Einsele H, Noppeney R, Hess G, Kallert S, et Al. (2016) Bispecific T-Cell Engager (BiTE) Antibody Construct Blinatumomab for the Treatment of Patients With Relapsed/Refractory Non-Hodgkin Lymphoma: Final Results From a Phase I Study. Journal of Clinical Oncology 34:10, 1104-1111; Jacoby E. (2019) Relapse and Resistance to CAR-T Cells and Blinatumomab in Hematologic Malignancies. Clin. Hematol. Int. 1(2):79-84; Nagorsen D, Kufer P, Baeuerle P A, Bargou R. (2012) Blinatumomab: a historical perspective. Pharmacol Ther. 136(3):335]. However, most patients relapse within 1-3 years depending on factors such as disease severity or number of prior treatments at treatment initiation [Myers R M, Taraseviciute A, Steinberg S M, et al. (2022) Blinatumomab Nonresponse and High-Disease Burden Are Associated with Inferior Outcomes After CD19-CAR for B-ALL. J Clin Oncol. 40(9):932-944; Braig F, Brandt A, Goebeler M, Tony H-P, Kurze A-K, Nollau P, et al. (2017) Resistance to anti-CD19/CD3 BiTE in acute lymphoblastic leukemia may be mediated by disrupted CD19 membrane trafficking. Blood 129:100-4; Sotillo, E., et Al (2015) Convergence of Acquired Mutations and Alternative Splicing of CD19 Enables Resistance to CART-19 Immunotherapy. Cancer Discov. 5(12): p. 1282-95; Wudhikarn K, King A C, Geyer M B, Roshal M, Bernal Y, Gyurkocza B, Perales M-A, Park J H. (2022) Outcomes of relapsed B-cell acute lymphoblastic leukemia after sequential treatment with blinatumomab and inotuzumab. Blood Advances. 6 (5): 1432-1443]. Additionally, escape strategies in response to CD19-targeted drugs are widely recognized in the clinic [Myers R M, Taraseviciute A, Steinberg S M, et al. (2022) Blinatumomab Nonresponse and High-Disease Burden Are Associated with Inferior Outcomes After CD19-CAR for B-ALL. J Clin Oncol. 40(9):932-944; Braig F, Brandt A, Goebeler M, Tony H-P, Kurze A-K, Nollau P, et al. (2017) Resistance to anti-CD19/CD3 BiTE in acute lymphoblastic leukemia may be mediated by disrupted CD19 membrane trafficking. Blood 129:100-4; Sotillo, E., et Al (2015) Convergence of Acquired Mutations and Alternative Splicing of CD19 Enables Resistance to CART-19 Immunotherapy. Cancer Discov. 5(12): p. 1282-95]. Therefore, alternative therapies, either as salvage courses, or potentially in combined regimens, are needed.

Numerous CD19- and CD20-targeted BiTEs have demonstrated killing efficacy across NHL/leukemia subtypes and cell lines [Liu L, Lam C K, Long V, Widjaja L, Yang Y, Li H, Jin L, Burke S, Gorlatov S, et. Al (2017) MGD011, A CD19 x CD3 Dual-Affinity Retargeting Bi-specific Molecule Incorporating Extended Circulating Half-life for the Treatment of B-Cell Malignancies. Clin Cancer Res. 23(6):1506-1518; Dreier T, Baeuerle P A, Fichtner Iduna, Grün M, Schlereth B, Lorenczewski G, et Al. (2003) T Cell Costimulus-Independent and Very Efficacious Inhibition of Tumor Growth in Mice Bearing Subcutaneous or Leukemic Human B Cell Lymphoma Xenografts by a CD19−/CD3− Bispecific Single-Chain Antibody Construct. J Immunol; 170:4397-4402; Rossi D L, Rossi E A, Cardillo T M, Goldenberg D M, Chang C H. (2014) A new class of bispecific antibodies to redirect T cells for cancer immunotherapy. mAbs, 6:2, 381-391, doi: 10.4161/mabs.27385; Mehta N K, Pfluegler M, Meetze K, et al. (2022) A novel IgGbased FLT3xCD3 bispecific antibody for the treatment of AML and B-ALL. Journal for ImmunoTherapy of Cancer. 10: e003882; Smith, E J et al. (2015) A novel, native-format bispecific antibody triggering T-cell killing of B-cells is robustly active in mouse tumor models and cynomolgus monkeys. Sci. Rep. 5; 17943; doi: 10.1038/srep17943; Wang S, Peng L, Xu W, Zhou Y, Zhu Z, Kong Y, Leung S, Wang J, Yan X, Mi J Q. (2022) Preclinical characterization and comparison between CD3/CD19 bispecific and novel CD3/CD19/CD20 trispecific antibodies against B-cell acute lymphoblastic leukemia: targeted immunotherapy for acute lymphoblastic leukemia. Front. Med. 16(1): 139-149].

There is both pre-clinical and clinical evidence in support of CD22-directed therapies in NHL and ALL settings [Yoshida, S, et Al. (2017) Efficacy of an anti-CD22 antibody-monomethyl auristatin E conjugate in a preclinical xenograft model of precursor B-cell acute lymphoblastic leukemia. Leuk. Lymphoma. 58(5):1254-1257; Kantarjian H M, DeAngelo D J, Stelljes M, et Al. (2019) Inotuzumab ozogamicin versus standard of care in relapsed or refractory acute lymphoblastic leukemia: final report and long-term survival follow-up from the randomized, phase 3 INO-VATE study. Cancer. 125(14):2474-2487; O'Donnell, R T, et Al. (2009) Treatment of non-Hodgkin's lymphoma xenografts with the HB22.7 anti-CD22 monoclonal antibody and phosphatase inhibitors improves efficacy. Cancer Immunol Immunother. 58(10):1715-22; O'Donnell, R T, et Al. (2009) Phosphatase inhibition augments anti-CD22-mediated signaling and cytotoxicity in non-hodgkin's lymphoma cells. Leuk Res. 33(7):964-9]. Indeed, CD22 is an attractive target due to its ability to internalize when bound by antibody, making it popular target for antibody drug conjugates [Haso W, Lee D W, Shah N N, Stetler-Stevenson M, Yuan C M, Pastan I H et al. (2013) Anti-CD22-chimeric antigen receptors targeting B-cell precursor acute lymphoblastic leukemia. Blood. 121(7):1165-74]. Also, the potential benefits for combining CD19 and CD22 (as well as CD20) in antibody and CAR-T format has been demonstrated in lymphoma models [24, 36, 37, 38, 40]. Still, there is no known use of BiTEs targeting CD22 and CD19, a novel strategy that was employed here. Interestingly, in vivo data suggests a possible benefit in mice receiving a combination treatment of BiTE 197 and blinatumomab, supporting the potential usefulness of our construct in a muti-targeted approach. Furthermore, the anti-CD22 scFv used in BiTE 197 has CD22 ligand blocking properties, which may prove efficacious based on previous studies that have shown superior tumoricidal effects when compared to non-ligand blocking anti-CD22 mAbs [O'Donnell, R T, et Al. (2009) Treatment of non-Hodgkin's lymphoma xenografts with the HB22.7 anti-CD22 monoclonal antibody and phosphatase inhibitors improves efficacy. Cancer Immunol Immunother. 58(10):1715-22].

In summation, the data demonstrates merit in utilizing CD22 as an alternate target antigen to CD19 or CD20 in a BiTE formulation for B-cell malignancies. Thus, this novel construct could be used as a primary, combination, or post-CD19 directed therapy.

It has been shown that a novel BiTE consisting of humanized anti-CD22 and anti-CD3 single chain variable fragments. Target binding of the anti-CD22 and anti-CD3 moieties was confirmed by flow cytometry. CD22-BiTE promoted in vitro cell-mediated cytotoxicity in a dose and effector:target (E:T)-dependent fashion. Additionally, in an established acute lymphoblastic leukemia (ALL) xenograft mouse model, CD22-BiTE demonstrated tumor growth inhibition, comparable to blinatumomab. Further, the combination of blinatumomab and CD22-BiTE yielded increased efficacy in vivo when compared to the single agents. In conclusion, we report here the development of a new BiTE with cytotoxic activity against CD22⁺ cells which could represent an alternate or complementary therapeutic option for B-cell malignancies.

In the production of our BiTEs, there may have been additional non-functional protein fragments or aggregates, evidenced by faint streaks in the coomassie blue stained SDS-PAGE gels. Additionally for this study, we concentrated purified protein from supernatant of mammalian cell culture without additional size-exclusion chromatography to remove non-functional protein fragments, thus likely producing a lower effective concentration for out BiTEs. No pharmacokinetic (PK) analysis was obtained in this study to quantify serum half-life. Previous studies with blinatumomab demonstrate serum clearance half-life in humans of 2.1 hours [Zhu M, Wu B, Brandl C, Johnson J, Wolf A, Chow A, Doshi S. (2016) Blinatumomab, a Bispecific T-cell Engager (BiTE(®)) for CD-19 Targeted Cancer Immunotherapy: Clinical Pharmacology and Its Implications. Clin Pharmacokinet. 55(10):1271-1288]. Future studies with our BiTE could investigate T-cell proliferation in vivo and perform PK studies to determine serum half-life. In our study, no maximum tolerable dose was reached, although no mice were euthanized from study from treatment-related side effects. Finally, as discussed, the BiTE 197 dosage in our in vivo studies was 10-fold higher than blina, but confounding differences in CD19 and CD22 expression and internalization suggest that a molar dosage equivalence may not be appropriate.

### Example 7: In Vivo

30 NOD/SCID mice (15 male/15 female) were divided into 5 groups (6 mice per group) as shown in Table 6 below.

TABLE 6
Treatment groups.

| Treatment | | | |
|---|---|---|---|
| Group | Designation | Treatment | Dosing |
| 1 | PBMC | PBMCs only + | Nalm-6 cell 10⁴ |
| | Control | PBS | cells/mouse |
| 2 | Positive | Blinatumomab + | PBMCs 10⁷ |
| | Control | PBMCs | cells/mouse |
| 3 | BiTE 197 | αCD3-αCD22 + | Blinatumomab 0.1 |
| | | PBMCs | μg/mouse |
| 4 | Tri-functional | αCD3-IL 15sushi- | BITE 197 1 μg/mouse |
| | Engager 301 | αCD22 + PBMCs | Tri-functional |
| 5 | Blinatumomab + | Blinatumomab + | Engager |
| | BiTE 197 | BiTE 197 + PBMCs | 301 1 μg/mouse |

Each mouse was administered 104 Nalm-6 cells via i.v. engraftment and PBMCs via i.v. injection with administration of the treatment via intravenous delivery through the tail vein immediately following engraftment and injection and at days 1, 2, 3 and 4. The mice generally weighed about 20 g which for blinatumomab equates to about 5 μg/Kg and about 50 μg/Kg of BiTE 197. An effector:T cell ratio of 3:1 was used. The method used in FIG. 11B was followed.

Survival of mice was monitored as shown in FIG. 14.

Levels of blinatumomab, BiTE 197 and Tri-functional Engager 301 were monitored.

### Example 8: Evaluation of Cytotoxic Effect and T-Cell Activation

Peripheral blood mononuclear effector cells (PBMCs) were co-incubated overnight with 50,000 CD22+ target Nalm-6 cells in the presence of BiTE-197, tri-functional engager 301 or an αCD22-IL-12-αCD3 tri-functional engager. Cells were incubated in 200 uL total volume in a 96-well round-bottom plate at the indicated effector:target ratios. Antibodies were present at a final concentration of 1 μg/mL, thus tri-functional engager 301 (102 KDa) and the IL-12-containing engager were present at ~½ the molar concentration of BiTE-197 (52 KDa). Cytotoxicity was determined via 7-AAD staining of target cells, as determined by flow cytometry. All measurements were performed in duplicate wells.

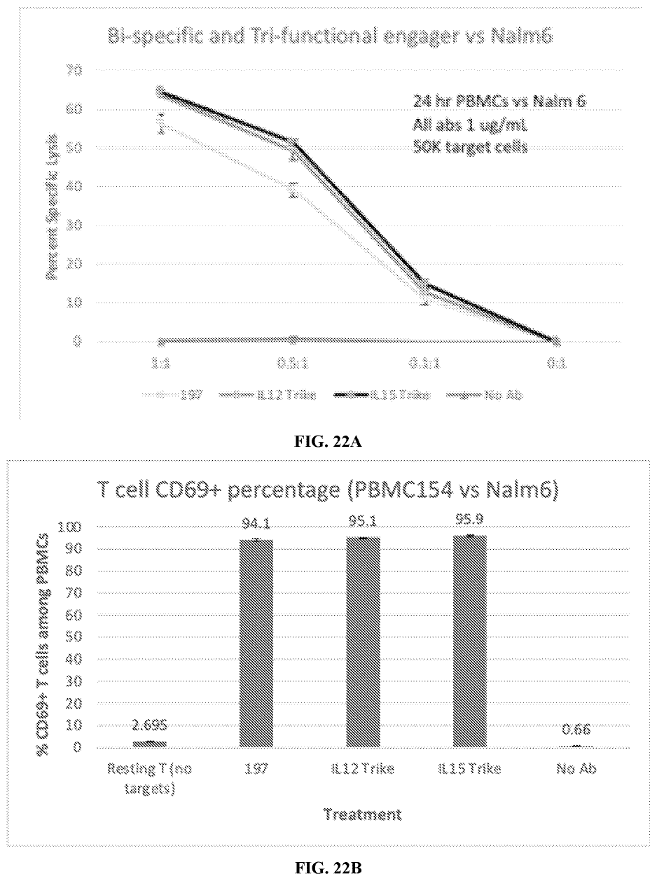

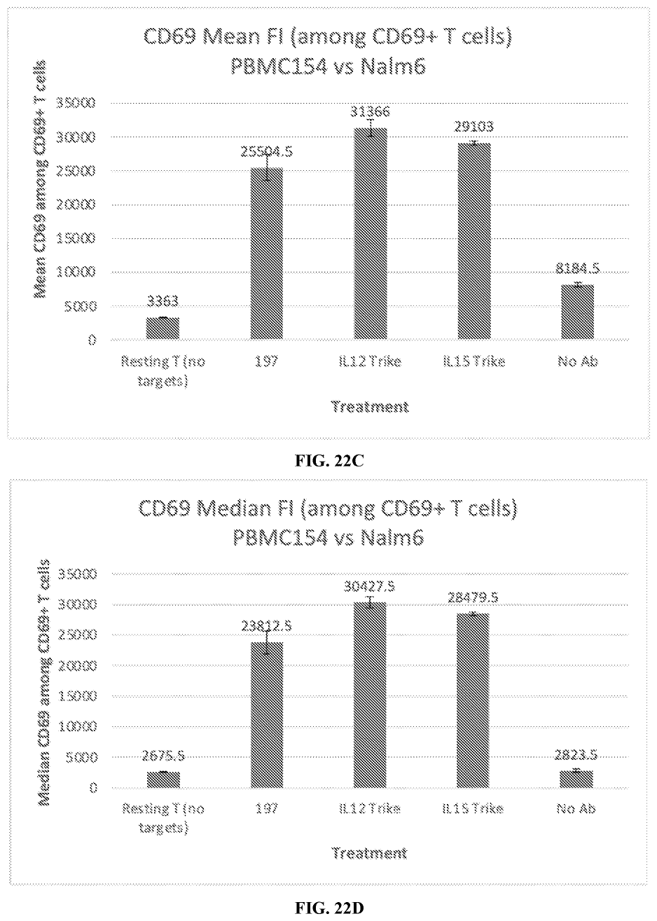

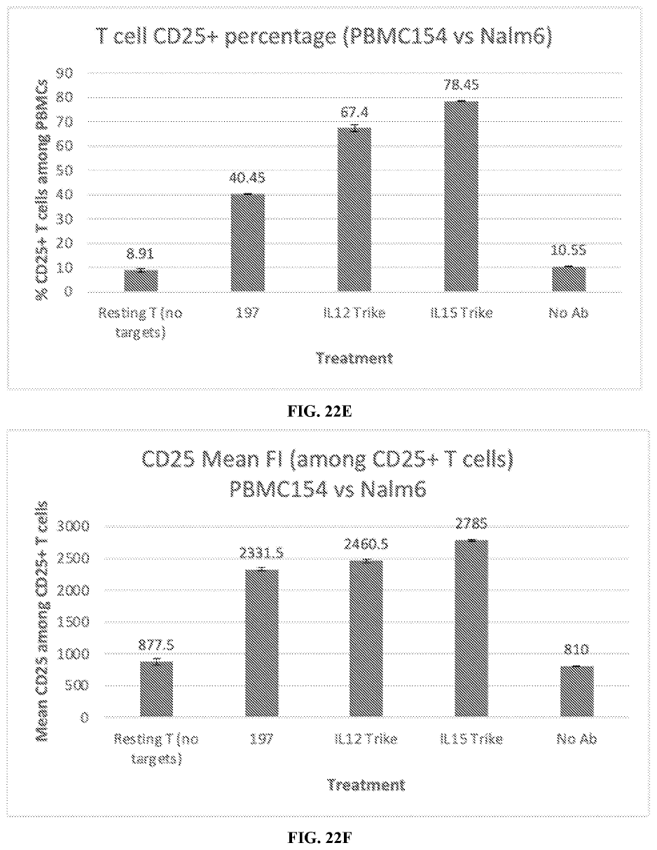

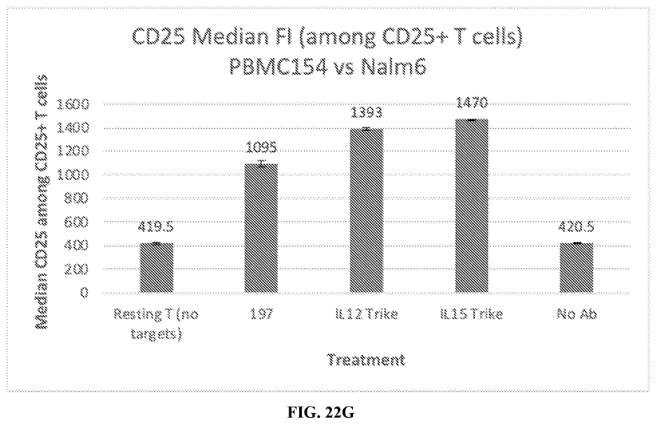

As shown in FIG. 22A, PBMCs, co-incubated overnight with CD22⁺ target Nalm-6 cells are able to lyse tumor cells upon addition of BiTE-197, tri-functional engager 301 or an αCD22-IL-12-αCD3 tri-functional engager. As further shown in FIG. 22A, the tri-functional engagers demonstrate superior cytotoxic capability compared to BiTE-197 and that the IL-12-containing tri-functional engager is at least as potent as the IL-15sushi tri-functional engager 301.

FIGS. 22B-22G show T-cell activation among the PBMCs following overnight incubation in the same assay in the wells with a 1:1 E:T ratio as measured by CD69 and CD25 expression. The IL-12-containing tri-functional engager demonstrated a superior increase in the CD69 activation marker (FIGS. 22C-22D).

### Example 9: Cytotoxicity in Combination with Anti-CD19 CAR-T Cells

BiTE-197, tri-functional engager 301, or the αCD22-IL-12-αCD3 tri-functional engager of Example 8 were incubated overnight with 50,000 CD19⁺/CD22⁺ target Nalm-6 target cells and standard-of-care anti-CD19 CAR-T effector cells ((αCD19-41BB-CD3z). The co-incubation was performed at the indicated E:T ratios in 200 μL of media in a round-bottom 96-well plate. The antibodies were used at a concentration of 1 μg/mL for BiTE-197 and 1.5 μg/mL for the two tri-functional engagers. Standard-of-care anti-CD19 CAR-T cells served as a control, as did target cells incubated alone. All conditions were assayed in duplicate, and cytotoxicity was determined using 7-AAD staining and assessment of the CellTraceViolet labeled target cells via flow cytometry.

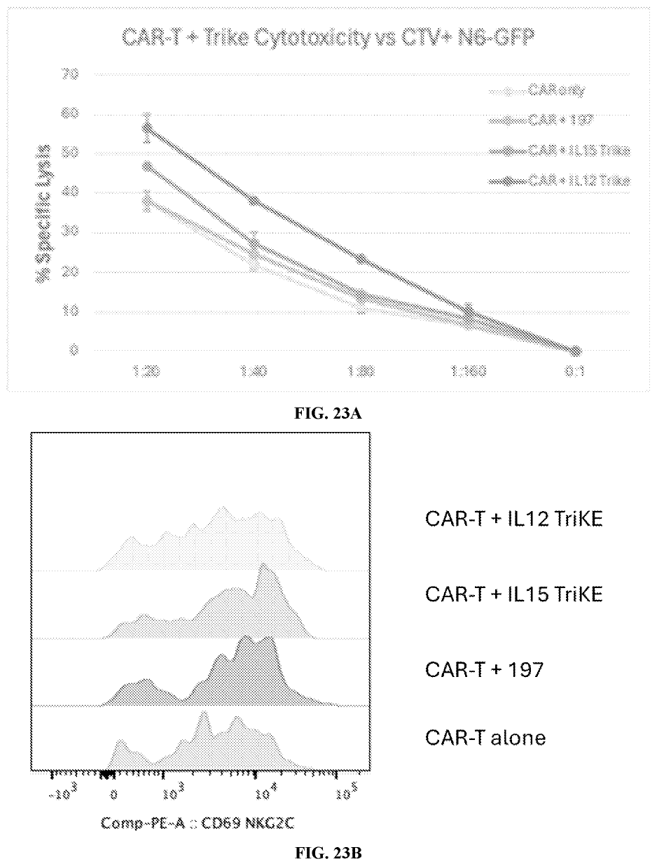

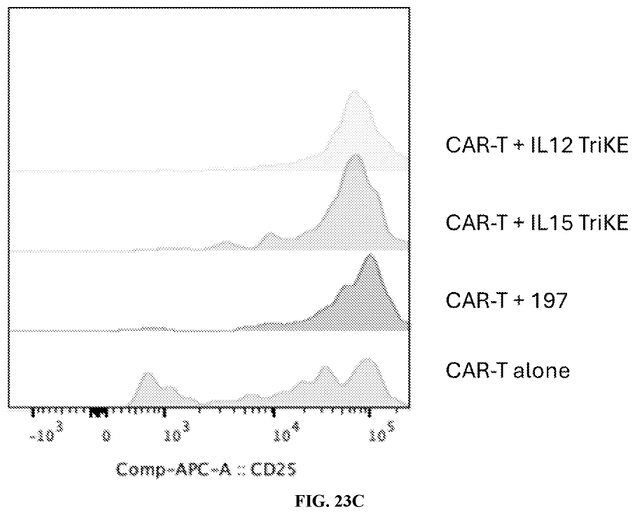

As shown in FIG. 23A, BiTE-197, tri-functional engager 301 and the αCD22-IL-12-αCD3 tri-functional engager enhanced T-cell mediated killing of the target cells. The αCD22-IL-12-αCD3 tri-functional engager was particularly effective, and was the best adjuvant at all E:T ratios tested, from 1:20 to 1:160. Data was obtained by flow cytometry in triplicate for each data point. FIGS. 23B-23C demonstrate that CD69 and CD25 activation marker levels in response to the four treatments.

### Example 10: Timecourse Study for Potentiation of CAR-T Cell Cytolytic Activity Via Addition of an αCD22-IL-12-αCD3 Tri-Functional Engager

50,000 Nalm-6-GFP target cells were co-incubated with the αCD22-IL-12-αCD3 tri-functional engager of Example 8 at 1.5 μg/mL and anti-CD19 CAR-T cells at a 1:40 E:T ratio for 36 hours. Cells were incubated in a flat-bottom 96-well plate. Data was generated using an Inucyte S3 Live Cell Imager.

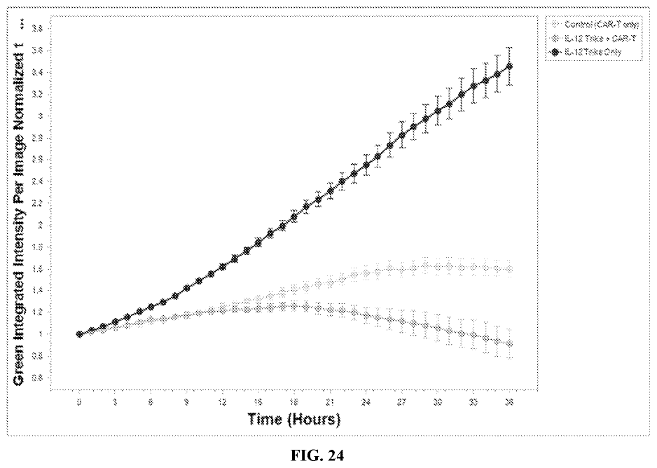

Data on cell viability represented by Green Integrated Intensity per image is shown in FIG. 24. As depicted in the figure, the addition of the αCD22-IL-12-αCD3 tri-functional engager to the wells with CAR-Ts and target cells improved the cytolytic potential of the CAR-Ts over a period of 36 hours, as compared to treatment with the antibody alone or CAR-Ts only. The green signal emitted from the GFP expressing target cells in the experimental wells, as normalized to the signal emitted per well at the start of the assay, shows that the CAR-T+antibody wells have the lowest normalized signal after 36 hours. This indicates superior in vitro tumor control by this treatment combination.

### Example 11: Timecourse Study for Comparative Cytotoxicity Mediated Antibody Constructs Upon Co-Incubation with PBMCs and Target Leukemia Cells

50,000 Nalm-6-GFP target cells were co-incubated with BiTE-197, tri-functional engager 301 or the αCD22-IL-12-αCD3 tri-functional engager of Example 8 at 1.0 μg/mL for the BiTE and 1.5 μg/mL for the tri-functional engagers and effector PBMCs at a 1:1 E:T ratio for 60 hours. Data was generated using an Inucyte S3 Live Cell Imager in triplicate. Cells were incubated in 200 μL of media in a flat-bottom 96-well plate.

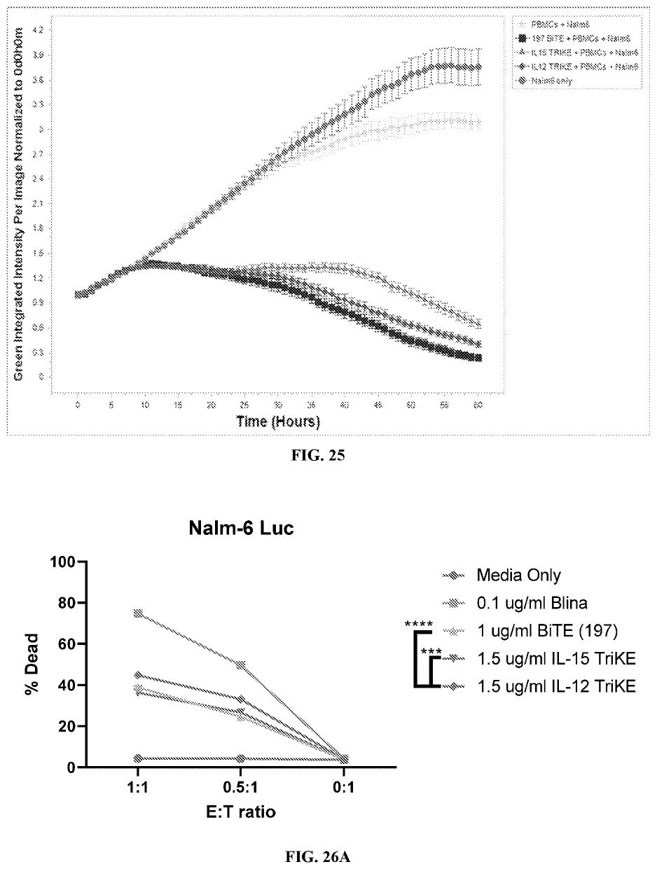

Data on cell viability represented by Green Integrated Intensity per image is shown in FIG. 25. Live Nalm6-GFP cells continue to generate GFP signal, while those that have been killed stop producing signal. All three antibodies exerted tumor cell cytolysis compared to untreated Nalm6 cells or those incubated only with PBMCs. Here, the BiTE-197 exhibited the best cytolytic potential, followed by the αCD22-IL-12-αCD3 and then the 302 tri-functional engagers.

### Example 12: Cytotoxicity Against Different Cell Types

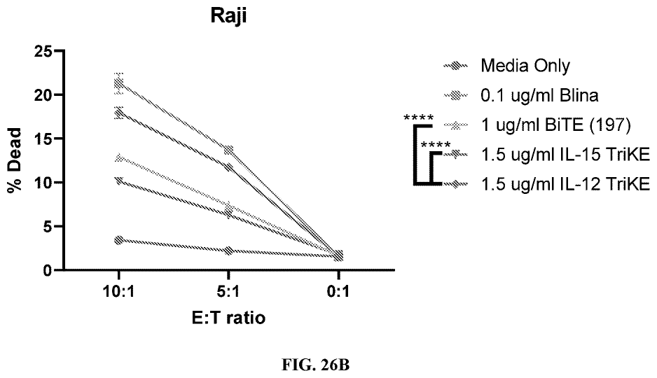

A cytotoxicity assay was performed using BiTE-197, tri-functional engager 301 or the αCD22-IL-12-αCD3 tri-functional engager of Example 8 co-incubated with PBMCs at different E:T ratios with 5×10⁵ Nalm6-GFP-Luc cells (FIG. 26A) or Raji cells (FIG. 26B) at different E:T ratios for 24 hours using blinatumomab as a control.

Killing was mediated via PBMCs co-incubated with the targets at the noted E:T ratios. The assay was run overnight, with cells maintained in 96-well round bottom plates in 200 μL/well, and killing was assessed using a flow cytometer with Zombie Near Infrared live-dead cell staining. The αCD22-IL-12-αCD3 tri-functional engager outperformed the other two antibodies in both assays at all E:Ts tested.

### Example 13: Sequences

Table 7 provides sequences referenced herein. Note an N-terminal methionine has not been introduced to accommodate translation in a bacterially-derived expression system for each distinct protein chain of the illustrative antibodies (e.g. V_{H} and V_{L} sequences).

It should be understood that sequences recited and claimed in this disclosure can be with or without an N-terminal methionine. For example, sequences below that do not include an N-terminal methionine can have one included and sequences that include an N-terminal methionine can be without the N-terminal methionine.

TABLE 7
Sequences. Unless otherwise noted, all sequences are amino acid
sequences.

| SEQ ID | | | | |
|---|---|---|---|---|
| NO: | Molecule | Region | Scheme | Sequence |
| 1 | HB22.7 VH | | | QVQLQESGPALVKPTQTLTLTCTVS |
| | | | | GFSLSDYGVNWIRQPPGKALEWLGI |
| | | | | IWGDGRTDYNSALKSRLSISKDNSK |
| | | | | NQVVLRMTNVDPVDTATYFCARAPG |
| | | | | NRAMEYWGQGTVVTVSSAST |
| 2 | HB22.7 | CDR-H3 | Kabat | APGNRAMEY |
| 3 | HB22.7 | CDR-H2 | Kabat | IIWGDGRTDYNSALKS |
| 4 | HB22.7 | CDR-H1 | Kabat | DYGVN |
| 5 | HB22.7 | CDR-H3 | Chothia | APGNRAMEY |
| 6 | HB22.7 | CDR-H2 | Chothia | WGDGR |
| 7 | HB22.7 | CDR-H1 | Chothia | GFSLSDY |
| 8 | HB22.7 VL | | | DIVMTQSPSSLSASVGDRVTITCKA |
| | | | | SQSVTNDVAWYQQKPGKAPKLLIYY |
| | | | | ASNRYTGVPDRFSGSGYGTDFTLTI |
| | | | | SSLQPEDFATYFCQQDYRSPWTFGG |
| | | | | GTKVEIK |
| 9 | HB22.7 | CDR-L3 | Kabat | QQDYRSPWT |
| 10 | HB22.7 | CDR-L2 | Kabat | YASNRYT |
| 11 | HB22.7 | CDR-L1 | Kabat | KASQSVTNDVA |
| 12 | HB22.7 | CDR-L3 | Chothia | QQDYRSPWT |
| 13 | HB22.7 | CDR-L2 | Chothia | YASNRYT |
| 14 | HB22.7 | CDR-L1 | Chothia | KASQSVTNDVA |
| 15 | HB22.7 scFv | | | DIVMTQSPSSLSASVGDRVTITCKA |
| | (VL-linker-VH) | | | SQSVTNDVAWYQQKPGKAPKLLIYY |
| | | | | ASNRYTGVPDRFSGSGYGTDFTLTI |
| | | | | SSLQPEDFATYFCQQDYRSPWTFGG |
| | | | | GTKVEIKGGGGSGGGGSGGGGSGGG |
| | | | | GSQVQLQESGPALVKPTQTLTLTCT |
| | | | | VSGFSLSDYGVNWIRQPPGKALEWL |
| | | | | GIIWGDGRTDYNSALKSRLSISKDN |
| | | | | SKNQVVLRMTNVDPVDTATYFCARA |
| | | | | PGNRAMEYWGQGTVVTVSSAST |
| 16 | TR66 VH | | | QVQLQQSGAELARPGASVKMSCKTS |
| | | | | GYTFTRYTMHWVKQRPGQGLEWIGY |
| | | | | INPSRGYTNYNQKFKDKATLTTDKS |
| | | | | SSTAYMQLSSLTSEDSAVYYCARYY |
| | | | | DDHYSLDYWGQGTTLTVSS |
| 17 | TR66 | CDR-H3 | Kabat | YYDDHYSLDY |
| 18 | TR66 | CDR-H2 | Kabat | YINPSRGYTNYNQKFKD |
| 19 | TR66 | CDR-H1 | Kabat | RYTMH |
| 20 | TR66 | CDR-H3 | Chothia | YYDDHYSLDY |
| 21 | TR66 | CDR-H2 | Chothia | NPSRGY |
| 22 | TR66 | CDR-H1 | Chothia | GYTFTRY |
| 23 | TR66 VL | | | QIVLTQSPAIMSASPGEKVTMTCRA |
| | | | | SSSVSYMNWYQQKSGTSPKRWIYDT |
| | | | | SKVASGVPYRFSGSGSGTSYSLTIS |
| | | | | SMEAEDAATYYCQQWSSNPLTFGAG |
| | | | | TKLELK |
| 24 | TR66 | CDR-L3 | Kabat | QQWSSNPLT |
| 25 | TR66 | CDR-L2 | Kabat | DTSKVAS |
| 26 | TR66 | CDR-L1 | Kabat | RASSSVSYMN |
| 27 | TR66 | CDR-L3 | Chothia | QQWSSNPLT |
| 28 | TR66 | CDR-L2 | Chothia | DTSKVAS |
| 29 | TR66 | CDR-L1 | Chothia | RASSSVSYMN |
| 30 | TR66 scFv | | | QVQLQQSGAELARPGASVKMSCKTS |
| | (VH-linker-VL) | | | GYTFTRYTMHWVKQRPGQGLEWIGY |
| | | | | INPSRGYTNYNQKFKDKATLTTDKS |
| | | | | SSTAYMQLSSLTSEDSAVYYCARYY |
| | | | | DDHYSLDYWGQGTTLTVSSGGGGSG |
| | | | | GGGSGGGGSGGGGSQIVLTQSPAIM |
| | | | | SASPGEKVTMTCRASSSVSYMNWYQ |
| | | | | QKSGTSPKRWIYDTSKVASGVPYRF |
| | | | | SGSGSGTSYSLTISSMEAEDAATYY |
| | | | | CQQWSSNPLTFGAGTKLELK |
| 31 | BiTE 197 | | | DIVMTQSPSSLSASVGDRVTITCKA |
| | | | | SQSVTNDVAWYQQKPGKAPKLLIYY |
| | | | | ASNRYTGVPDRFSGSGYGTDFTLTI |
| | | | | SSLQPEDFATYFCQQDYRSPWTFGG |
| | | | | GTKVEIKGGGGSGGGGSGGGGSGGG |
| | | | | GSQVQLQESGPALVKPTQTLTLTCT |
| | | | | VSGFSLSDYGVNWIRQPPGKALEWL |
| | | | | GIIWGDGRTDYNSALKSRLSISKDN |
| | | | | SKNQVVLRMTNVDPVDTATYFCARA |
| | | | | PGNRAMEYWGQGTVVTVSSASTSGG |
| | | | | CGGSQVQLQQSGAELARPGASVKMS |
| | | | | CKTSGYTFTRYTMHWVKQRPGQGLE |
| | | | | WIGYINPSRGYTNYNQKFKDKATLT |
| | | | | TDKSSSTAYMQLSSLTSEDSAVYYC |
| | | | | ARYYDDHYSLDYWGQGTTLTVSSGG |
| | | | | GGSGGGGSGGGGSGGGGSQIVLTQS |
| | | | | PAIMSASPGEKVTMTCRASSSVSYM |
| | | | | NWYQQKSGTSPKRWIYDTSKVASGV |
| | | | | PYRFSGSGSGTSYSLTISSMEAEDA |
| | | | | ATYYCQQWSSNPLTFGAGTKLELK |
| 32 | sushi domain | | | ITCPPPMSVEHADIWVKSYSLYSRE |
| | (aa 31-95 of IL- | | | RYICNSGFKRKAGTSSLTECVLNKA |
| | 15Rα) | | | TNVAHWTTPSLKCIR |
| 33 | sushi domain (aa | | | ITCPPPMSVEHADIWVKSYSLYSRE |
| | 31-107 of IL- | | | RYICNSGFKRKAGTSSLTECVLNKA |
| | 15Rα) | | | TNVAHWTTPSLKCIRDPALVHQRPA |
| | | | | PP |
| 34 | human IL-15 | | | MRISKPHLRSISIQCYLCLLLNSHF |
| | (P40933) | | | LTEAGIHVFILGCFSAGLPKTEANW |
| | | | | VNVISDLKKIEDLIQSMHIDATLYT |
| | | | | ESDVHPSCKVTAMKCFLLELQVISL |
| | | | | ESGDASIHDTVENLIILANNSLSSN |
| | | | | GNVTESGCKECEELEEKNIKEFLQS |
| | | | | FVHIVQMFINTS |
| 35 | human IL-15 | | | NWVNVISDLKKIEDLIQSMHIDATL |
| | | | | YTESDVHPSCKVTAMKCFLLELQVI |
| | | | | SLESGDASIHDTVENLIILANNSLS |
| | | | | SNGNVTESGCKECEELEEKNIKEFL |
| | | | | QSFVHIVQMFINTS |
| 36 | IL-15 mutein | | | NWVNVISDLKKIEDLIQSMHIDATL |
| | (N72D) | | | YTESDVHPSCKVTAMKCFLLELQVI |
| | | | | SLESGDASIHDTVENLIILANDSLS |
| | | | | SNGNVTESGCKECEELEEKNIKEFL |
| | | | | QSFVHIVQMFINTS |
| 37 | sushi domain-IL- | | | ITCPPPMSVEHADIWVKSYSLYSRE |
| | 15 fusion | | | RYICNSGFKRKAGTSSLTECVLNKA |
| | including linker | | | TNVAHWTTPSLKCIRDPALVHQRPA |
| | | | | PPGGGSGGGGSGGGSGGGGSLQNWV |
| | | | | NVISDLKKIEDLIQSMHIDATLYTE |
| | | | | SDVHPSCKVTAMKCFLLELQVISLE |
| | | | | SGDASIHDTVENLIILANNSLSSNG |
| | | | | NVTESGCKECEELEEKNIKEFLQSF |
| | | | | VHIVQMFINTS |
| 38 | (G4S)₄ linker | | | GGGGSGGGGSGGGGSGGGGS |
| 39 | Linker | | | GGGGSGGGGSCGGGGS |
| 40 | Linker | | | GGGGSGGGGCGGGGSGGGGS |
| 41 | Linker | | | SGGCGGS |
| 42 | Linker | | | SG₄S |
| 43 | Linker | | | (G₄S)₃ |
| 44 | Linker | | | (G₄S)₂C(G₄S)₂ |
| 45 | Linker | | | ASTGGS |
| 46 | Linker | | | (S_{G}4)₄SG |
| 47 | Linker | | | GSTSGSGKPGSGEGSTKG |
| 48 | Linker | | | PSGQAGAAASESLFVSNHAY |
| 49 | Linker | | | EASGGPE |
| 50 | Linker | | | (G₄S)₂ |
| 51 | Linker | | | G₃SG₄SG₃SG₄SLQ |
| 52 | Tri-functional | | | DIVMTQSPSSLSASVGDRVTITCKA |
| | Engager 301 | | | SQSVTNDVAWYQQKPGKAPKLLIYY |
| | | | | ASNRYTGVPDRFSGSGYGTDFTLTI |
| | | | | SSLQPEDFATYFCQQDYRSPWTFGG |
| | | | | GTKVEIKGGGGSGGGGSGGGGSGGG |
| | | | | GSQVQLQESGPALVKPTQTLTLTCT |
| | | | | VSGFSLSDYGVNWIRQPPGKALEWL |
| | | | | GIIWGDGRTDYNSALKSRLSISKDN |
| | | | | SKNQVVLRMTNVDPVDTATYFCARA |
| | | | | PGNRAMEYWGQGTVVTVSSASTSGG |
| | | | | GGSGGGGSGGGGSGGGGSGITCPPP |
| | | | | MSVEHADIWVKSYSLYSRERYICNS |
| | | | | GFKRKAGTSSLTECVLNKATNVAHW |
| | | | | TTPSLKCIRDPALVHQRPAPPGGGS |
| | | | | GGGGSGGGSGGGGSLQNWVNVISDL |
| | | | | KKIEDLIQSMHIDATLYTESDVHPS |
| | | | | CKVTAMKCFLLELQVISLESGDASI |
| | | | | HDTVENLIILANNSLSSNGNVTESG |
| | | | | CKECEELEEKNIKEFLQSFVHIVQM |
| | | | | FINTSGSTSGSGKPGSGEGSTKGQV |
| | | | | QLQQSGAELARPGASVKMSCKTSGY |
| | | | | TFTRYTMHWVKQRPGQGLEWIGYIN |
| | | | | PSRGYTNYNQKFKDKATLTTDKSSS |
| | | | | TAYMQLSSLTSEDSAVYYCARYYDD |
| | | | | HYSLDYWGQGTTLTVSSGGGGSGGG |
| | | | | GSGGGGSGGGGSQIVLTQSPAIMSA |
| | | | | SPGEKVTMTCRASSSVSYMNWYQQK |
| | | | | SGTSPKRWIYDTSKVASGVPYRFSG |
| | | | | SGSGTSYSLTISSMEAEDAATYYCQ |
| | | | | QWSSNPLTFGAGTKLELK |
| 53 | Linker | | | GGS |
| 54 | Tri-Functional | | | ATGGACATGAGGGTCCCTGCTCAGC |
| | Engager 301 | | | TCCTGGGGCTCCTGCAGCTCTGGCT |
| | (includes signal | | | CTCAGGTGCCAGATGTGACATCGTG |
| | peptide and C- | | | ATGACCCAGTCTCCATCCTCCCTGT |
| | terminal GGS | | | CTGCATCTGTAGGAGACAGAGTCAC |
| | linker-His tag) | | | CATCACTTGCAAGGCCAGTCAGAGT |
| | (nucleotide | | | GTGACTAATGATGTAGCTTGGTATC |
| | sequence) | | | AGCAGAAACCAGGGAAAGCCCCTAA |
| | | | | GCTCCTGATCTACTATGCATCCAAT |
| | | | | CGCTACACTGGGGTCCCAGATAGGT |
| | | | | TCAGTGGAAGTGGATATGGGACAGA |
| | | | | TTTTACTTTGACCATCAGCAGCCTG |
| | | | | CAGCCTGAAGATTTTGCAACATATT |
| | | | | TCTGTCAGCAGGATTATAGGTCTCC |
| | | | | GTGGACGTTTGGCGGCGGTACCAAA |
| | | | | GTGGAGATCAAAGGCGGCGGCGGAT |
| | | | | CTGGTGGCGGAGGGTCAGGGGGAGG |
| | | | | CGGTAGTGGTGGCGGAGGGAGCCAG |
| | | | | GTCCAGTTGCAGGAGTCTGGTCCTG |
| | | | | CGCTGGTGAAACCCACACAGACCCT |
| | | | | CACACTGACCTGCACCGTGTCTGGG |
| | | | | TTCTCACTCAGCGACTATGGTGTAA |
| | | | | ACTGGATCCGTCAGCCCCCAGGGAA |
| | | | | GGCCCTGGAGTGGCTTGGAATAATA |
| | | | | TGGGGTGATGGAAGGACAGACTATA |
| | | | | ATTCAGCTCTCAAATCCAGGCTCAG |
| | | | | CATCTCCAAGGACAACTCCAAAAAC |
| | | | | CAGGTGGTCCTTAGAATGACCAACG |
| | | | | TGGACCCTGTGGACACAGCCACGTA |
| | | | | TTTCTGTGCAAGAGCCCCCGGTAAT |
| | | | | AGGGCTATGGAGTACTGGGGCCAGG |
| | | | | GAACCGTGGTCACCGTCTCCTCAGC |
| | | | | CTCCACCTCCGGCGGCGGAGGAAGC |
| | | | | GGTGGGGGCGGTTCTGGAGGCGGTG |
| | | | | GTAGTGGCGGAGGAGGCAGTGGAAT |
| | | | | CACGTGCCCTCCCCCCATGTCCGTG |
| | | | | GAACACGCAGACATCTGGGTCAAGA |
| | | | | GCTACAGCTTGTACTCCAGGGAGCG |
| | | | | GTACATTTGTAACTCTGGTTTCAAG |
| | | | | CGTAAAGCCGGCACGTCCAGCCTGA |
| | | | | CGGAGTGCGTGTTGAACAAGGCCAC |
| | | | | GAATGTCGCCCACTGGACAACCCCC |
| | | | | AGTCTCAAATGCATTAGAGACCCTG |
| | | | | CCCTGGTTCACCAAAGGCCAGCGCC |
| | | | | ACCCGGCGGAGGATCAGGCGGGGGC |
| | | | | GGTAGTGGTGGCGGTTCTGGTGGTG |
| | | | | GAGGTTCATTGCAAAATTGGGTAAA |
| | | | | TGTGATCTCTGACCTGAAGAAGATT |
| | | | | GAGGATCTCATCCAATCTATGCATA |
| | | | | TCGACGCCACCCTCTATACCGAAAG |
| | | | | TGATGTCCACCCCAGCTGCAAGGTA |
| | | | | ACAGCAATGAAATGTTTTCTGCTCG |
| | | | | AACTTCAAGTGATATCCTTGGAATC |
| | | | | AGGCGACGCATCAATCCATGATACA |
| | | | | GTCGAAAATCTCATCATTCTGGCAA |
| | | | | ACAATTCCCTCTCCAGCAATGGCAA |
| | | | | CGTCACTGAGTCAGGATGCAAAGAG |
| | | | | TGCGAAGAGCTCGAAGAAAAGAATA |
| | | | | TCAAGGAGTTCCTGCAAAGCTTTGT |
| | | | | TCATATCGTCCAAATGTTCATTAAT |
| | | | | ACATCCGGGAGTACGTCAGGATCTG |
| | | | | GGAAGCCAGGCTCAGGAGAGGGCTC |
| | | | | TACAAAGGGACAGGTGCAGCTGCAG |
| | | | | CAGTCTGGCGCTGAGCTGGCTAGAC |
| | | | | CTGGCGCCTCCGTGAAGATGTCCTG |
| | | | | CAAGACCTCCGGCTACACCTTCACC |
| | | | | CGGTACACCATGCACTGGGTCAAGC |
| | | | | AGAGGCCTGGACAGGGCCTGGAATG |
| | | | | GATCGGCTACATCAACCCCTCCCGG |
| | | | | GGCTACACCAACTACAACCAGAAGT |
| | | | | TCAAGGACAAGGCCACCCTGACAAC |
| | | | | CGACAAGTCCTCCTCCACCGCCTAC |
| | | | | ATGCAGCTGTCCTCCCTGACCTCCG |
| | | | | AGGACTCCGCCGTGTACTACTGCGC |
| | | | | CCGGTACTACGACGACCACTACTCC |
| | | | | CTGGACTACTGGGGCCAGGGCACCA |
| | | | | CACTGACAGTGTCTAGCGGAGGCGG |
| | | | | AGGATCTGGTGGTGGCGGATCTGGC |
| | | | | GGCGGTGGAAGTGGCGGAGGTGGTA |
| | | | | GCCAGATCGTGCTGACCCAGTCTCC |
| | | | | CGCCATCATGTCTGCTAGCCCTGGC |
| | | | | GAGAAAGTGACAATGACCTGCCGGG |
| | | | | CCTCCTCCTCCGTGTCCTACATGAA |
| | | | | CTGGTATCAGCAGAAGTCCGGCACC |
| | | | | TCCCCCAAGCGGTGGATCTACGACA |
| | | | | CCTCCAAGGTGGCCTCTGGCGTGCC |
| | | | | CTACAGATTCTCCGGCTCTGGCTCT |
| | | | | GGCACCTCCTACAGCCTGACCATCT |
| | | | | CCAGCATGGAAGCCGAGGATGCCGC |
| | | | | CACCTACTACTGCCAGCAGTGGTCC |
| | | | | TCCAACCCCCTGACCTTTGGCGCTG |
| | | | | GCACCAAGCTGGAACTGAAGGGCGG |
| | | | | CTCTCATCACCATCATCACCATTGA |
| 55 | CD22 | | | MHLLGPWLLLLVLEYLAFSDSSKWV |
| | | | | FEHPETLYAWEGACVWIPCTYRALD |
| | | | | GDLESFILFHNPEYNKNTSKFDGTR |
| | | | | LYESTKDGKVPSEQKRVQFLGDKNK |
| | | | | NCTLSIHPVHLNDSGQLGLRMESKT |
| | | | | EKWMERIHLNVSERPFPPHIQLPPE |
| | | | | IQESQEVTLTCLLNFSCYGYPIQLQ |
| | | | | WLLEGVPMRQAAVTSTSLTIKSVFT |
| | | | | RSELKFSPQWSHHGKIVTCQLQDAD |
| | | | | GKFLSNDTVQLNVKHTPKLEIKVTP |
| | | | | SDAIVREGDSVTMTCEVSSSNPEYT |
| | | | | TVSWLKDGTSLKKQNTFTLNLREVT |
| | | | | KDQSGKYCCQVSNDVGPGRSEEVFL |
| | | | | QVQYAPEPSTVQILHSPAVEGSQVE |
| | | | | FLCMSLANPLPTNYTWYHNGKEMQG |
| | | | | RTEEKVHIPKILPWHAGTYSCVAEN |
| | | | | ILGTGQRGPGAELDVQYPPKKVTTV |
| | | | | IQNPMPIREGDTVTLSCNYNSSNPS |
| | | | | VTRYEWKPHGAWEEPSLGVLKIQNV |
| | | | | GWDNTTIACAACNSWCSWASPVALN |
| | | | | VQYAPRDVRVRKIKPLSEIHSGNSV |
| | | | | SLQCDFSSSHPKEVQFFWEKNGRLL |
| | | | | GKESQLNEDSISPEDAGSYSCWVNN |
| | | | | SIGQTASKAWTLEVLYAPRRLRVSM |
| | | | | SPGDQVMEGKSATLTCESDANPPVS |
| | | | | HYTWFDWNNQSLPYHSQKLRLEPVK |
| | | | | VQHSGAYWCQGTNSVGKGRSPLSTL |
| | | | | TVYYSPETIGRRVAVGLGSCLAILI |
| | | | | LAICGLKLQRRWKRTQSQQGLQENS |
| | | | | SGQSFFVRNKKVRRAPLSEGPHSLG |
| | | | | CYNPMMEDGISYTTLRFPEMNIPRT |
| | | | | GDAESSEMQRPPPDCDDTVTYSALH |
| | | | | KRQVGDYENVIPDFPEDEGIHYSEL |
| | | | | IQFGVGERPQAQENVDYVILKH |
| 56 | CD3E | | | MQSGTHWRVLGLCLLSVGVWGQDGN |
| | | | | EEMGGITQTPYKVSISGTTVILTCP |
| | | | | QYPGSEILWQHNDKNIGGDEDDKNI |
| | | | | GSDEDHLSLKEFSELEQSGYYVCYP |
| | | | | RGSKPEDANFYLYLRARVCENCMEM |
| | | | | DVMSVATIVIVDICITGGLLLLVYY |
| | | | | WSKNRKAKAKPVTRGAGAGGRQRGQ |
| | | | | NKERPPPVPNPDYEPIRKGQRDLYS |
| | | | | GLNQRRI |
| 57 | BiTE 197 | | | MDMRVPAQLLGLLQLWLSGARCDIV |
| | (including signal | | | MTQSPSSLSASVGDRVTITCKASQS |
| | sequence and C- | | | VTNDVAWYQQKPGKAPKLLIYYASN |
| | terminal GGS | | | RYTGVPDRFSGSGYGTDFTLTISSL |
| | linker-His tag) | | | QPEDFATYFCQQDYRSPWTFGGGTK |
| | | | | VEIKGGGGSGGGGSGGGGSGGGGSQ |
| | | | | VQLQESGPALVKPTQTLTLTCTVSG |
| | | | | FSLSDYGVNWIRQPPGKALEWLGII |
| | | | | WGDGRTDYNSALKSRLSISKDNSKN |
| | | | | QVVLRMTNVDPVDTATYFCARAPGN |
| | | | | RAMEYWGQGTVVTVSSASTSGGCGG |
| | | | | SQVQLQQSGAELARPGASVKMSCKT |
| | | | | SGYTFTRYTMHWVKQRPGQGLEWIG |
| | | | | YINPSRGYTNYNQKFKDKATLTTDK |
| | | | | SSSTAYMQLSSLTSEDSAVYYCARY |
| | | | | YDDHYSLDYWGQGTTLTVSSGGGGS |
| | | | | GGGGSGGGGSGGGGSQIVLTQSPAI |
| | | | | MSASPGEKVTMTCRASSSVSYMNWY |
| | | | | QQKSGTSPKRWIYDTSKVASGVPYR |
| | | | | FSGSGSGTSYSLTISSMEAEDAATY |
| | | | | YCQQWSSNPLTFGAGTKLELKGGSH |
| | | | | HHHHH* |
| 58 | BiTE 197 | | | ATGGACATGAGGGTCCCTGCTCAGC |
| | (including signal | | | TCCTGGGGCTCCTGCAGCTCTGGCT |
| | sequence and C- | | | CTCAGGTGCCAGATGTGACATCGTG |
| | terminal His tag) | | | ATGACCCAGTCTCCATCCTCCCTGT |
| | (nucleotide | | | CTGCATCTGTAGGAGACAGAGTCAC |
| | sequence) | | | CATCACTTGCAAGGCCAGTCAGAGT |
| | | | | GTGACTAATGATGTAGCTTGGTATC |
| | | | | AGCAGAAACCAGGGAAAGCCCCTAA |
| | | | | GCTCCTGATCTACTATGCATCCAAT |
| | | | | CGCTACACTGGGGTCCCAGATAGGT |
| | | | | TCAGTGGAAGTGGATATGGGACAGA |
| | | | | TTTTACTTTGACCATCAGCAGCCTG |
| | | | | CAGCCTGAAGATTTTGCAACATATT |
| | | | | TCTGTCAGCAGGATTATAGGTCTCC |
| | | | | GTGGACGTTTGGCGGCGGTACCAAA |
| | | | | GTGGAGATCAAAGGCGGCGGCGGAT |
| | | | | CTGGTGGCGGAGGGTCAGGGGGAGG |
| | | | | CGGTAGTGGTGGCGGAGGGAGCCAG |
| | | | | GTCCAGTTGCAGGAGTCTGGTCCTG |
| | | | | CGCTGGTGAAACCCACACAGACCCT |
| | | | | CACACTGACCTGCACCGTGTCTGGG |
| | | | | TTCTCACTCAGCGACTATGGTGTAA |
| | | | | ACTGGATCCGTCAGCCCCCAGGGAA |
| | | | | GGCCCTGGAGTGGCTTGGAATAATA |
| | | | | TGGGGTGATGGAAGGACAGACTATA |
| | | | | ATTCAGCTCTCAAATCCAGGCTCAG |
| | | | | CATCTCCAAGGACAACTCCAAAAAC |
| | | | | CAGGTGGTCCTTAGAATGACCAACG |
| | | | | TGGACCCTGTGGACACAGCCACGTA |
| | | | | TTTCTGTGCAAGAGCCCCCGGTAAT |
| | | | | AGGGCTATGGAGTACTGGGGCCAGG |
| | | | | GAACCGTGGTCACCGTCTCCTCAGC |
| | | | | CTCCACCAGTGGCGGCTGTGGAGGA |
| | | | | TCCCAGGTGCAGCTGCAGCAGTCTG |
| | | | | GCGCTGAGCTGGCTAGACCTGGCGC |
| | | | | CTCCGTGAAGATGTCCTGCAAGACC |
| | | | | TCCGGCTACACCTTCACCCGGTACA |
| | | | | CCATGCACTGGGTCAAGCAGAGGCC |
| | | | | TGGACAGGGCCTGGAATGGATCGGC |
| | | | | TACATCAACCCCTCCCGGGGCTACA |
| | | | | CCAACTACAACCAGAAGTTCAAGGA |
| | | | | CAAGGCCACCCTGACAACCGACAAG |
| | | | | TCCTCCTCCACCGCCTACATGCAGC |
| | | | | TGTCCTCCCTGACCTCCGAGGACTC |
| | | | | CGCCGTGTACTACTGCGCCCGGTAC |
| | | | | TACGACGACCACTACTCCCTGGACT |
| | | | | ACTGGGGCCAGGGCACCACACTGAC |
| | | | | AGTGTCTAGCGGAGGCGGAGGATCT |
| | | | | GGTGGTGGCGGATCTGGCGGCGGTG |
| | | | | GAAGTGGCGGAGGTGGTAGCCAGAT |
| | | | | CGTGCTGACCCAGTCTCCCGCCATC |
| | | | | ATGTCTGCTAGCCCTGGCGAGAAAG |
| | | | | TGACAATGACCTGCCGGGCCTCCTC |
| | | | | CTCCGTGTCCTACATGAACTGGTAT |
| | | | | CAGCAGAAGTCCGGCACCTCCCCCA |
| | | | | AGCGGTGGATCTACGACACCTCCAA |
| | | | | GGTGGCCTCTGGCGTGCCCTACAGA |
| | | | | TTCTCCGGCTCTGGCTCTGGCACCT |
| | | | | CCTACAGCCTGACCATCTCCAGCAT |
| | | | | GGAAGCCGAGGATGCCGCCACCTAC |
| | | | | TACTGCCAGCAGTGGTCCTCCAACC |
| | | | | CCCTGACCTTTGGCGCTGGCACCAA |
| | | | | GCTGGAACTGAAGGGCGGCTCTCAC |
| | | | | CACCACCATCACCACTGA |
| 59 | BiTE 197/Tri- | | | MDMRVPAQLLGLLQLWLSGARC |
| | functional | | | |
| | Engager | | | |
| | 301/BiTE 198 | | | |
| | signal sequence | | | |
| 60 | BiTE 197/Tri- | | | ATGGACATGAGGGTCCCTGCTCAGC |
| | functional | | | TCCTGGGGCTCCTGCAGCTCTGGCT |
| | Engager | | | CTCAGGTGCCAGATGT |
| | 301/BiE 198 | | | |
| | signal sequence | | | |
| | (nucleotide | | | |
| | sequence) | | | |
| 61 | HB22.7 VL | | | GACATCGTGATGACCCAGTCTCCAT |
| | (nucleotide | | | CCTCCCTGTCTGCATCTGTAGGAGA |
| | sequence) | | | CAGAGTCACCATCACTTGCAAGGCC |
| | | | | AGTCAGAGTGTGACTAATGATGTAG |
| | | | | CTTGGTATCAGCAGAAACCAGGGAA |
| | | | | AGCCCCTAAGCTCCTGATCTACTAT |
| | | | | GCATCCAATCGCTACACTGGGGTCC |
| | | | | CAGATAGGTTCAGTGGAAGTGGATA |
| | | | | TGGGACAGATTTTACTTTGACCATC |
| | | | | AGCAGCCTGCAGCCTGAAGATTTTG |
| | | | | CAACATATTTCTGTCAGCAGGATTA |
| | | | | TAGGTCTCCGTGGACGTTTGGCGGC |
| | | | | GGTACCAAAGTGGAGATCAAA |
| 62 | (G₄S)₄ linker | | | GGCGGCGGCGGATCTGGTGGCGGAG |
| | (nucleotide | | | GGTCAGGGGGAGGCGGTAGTGGTGG |
| | sequence) | | | CGGAGGGAGC |
| 63 | HB22.7 VH | | | CAGGTCCAGTTGCAGGAGTCTGGTC |
| | (nucleotide | | | CTGCGCTGGTGAAACCCACACAGAC |
| | sequence) | | | CCTCACACTGACCTGCACCGTGTCT |
| | | | | GGGTTCTCACTCAGCGACTATGGTG |
| | | | | TAAACTGGATCCGTCAGCCCCCAGG |
| | | | | GAAGGCCCTGGAGTGGCTTGGAATA |
| | | | | ATATGGGGTGATGGAAGGACAGACT |
| | | | | ATAATTCAGCTCTCAAATCCAGGCT |
| | | | | CAGCATCTCCAAGGACAACTCCAAA |
| | | | | AACCAGGTGGTCCTTAGAATGACCA |
| | | | | ACGTGGACCCTGTGGACACAGCCAC |
| | | | | GTATTTCTGTGCAAGAGCCCCCGGT |
| | | | | AATAGGGCTATGGAGTACTGGGGCC |
| | | | | AGGGAACCGTGGTCACCGTCTCCTC |
| | | | | AGCCTCCACC |
| 64 | Linker | | | AGTGGCGGCTGTGGAGGATCC |
| | SGGCGGS | | | |
| | (nucleotide | | | |
| | sequence) | | | |
| 65 | TR66 VH | | | CAGGTGCAGCTGCAGCAGTCTGGCG |
| | (nucleotide | | | CTGAGCTGGCTAGACCTGGCGCCTC |
| | sequence) | | | CGTGAAGATGTCCTGCAAGACCTCC |
| | | | | GGCTACACCTTCACCCGGTACACCA |
| | | | | TGCACTGGGTCAAGCAGAGGCCTGG |
| | | | | ACAGGGCCTGGAATGGATCGGCTAC |
| | | | | ATCAACCCCTCCCGGGGCTACACCA |
| | | | | ACTACAACCAGAAGTTCAAGGACAA |
| | | | | GGCCACCCTGACAACCGACAAGTCC |
| | | | | TCCTCCACCGCCTACATGCAGCTGT |
| | | | | CCTCCCTGACCTCCGAGGACTCCGC |
| | | | | CGTGTACTACTGCGCCCGGTACTAC |
| | | | | GACGACCACTACTCCCTGGACTACT |
| | | | | GGGGCCAGGGCACCACACTGACAGT |
| | | | | GTCTAGC |
| 66 | TR66 VL | | | CAGATCGTGCTGACCCAGTCTCCCG |
| | (nucleotide | | | CCATCATGTCTGCTAGCCCTGGCGA |
| | sequence) | | | GAAAGTGACAATGACCTGCCGGGCC |
| | | | | TCCTCCTCCGTGTCCTACATGAACT |
| | | | | GGTATCAGCAGAAGTCCGGCACCTC |
| | | | | CCCCAAGCGGTGGATCTACGACACC |
| | | | | TCCAAGGTGGCCTCTGGCGTGCCCT |
| | | | | ACAGATTCTCCGGCTCTGGCTCTGG |
| | | | | CACCTCCTACAGCCTGACCATCTCC |
| | | | | AGCATGGAAGCCGAGGATGCCGCCA |
| | | | | CCTACTACTGCCAGCAGTGGTCCTC |
| | | | | CAACCCCCTGACCTTTGGCGCTGGC |
| | | | | ACCAAGCTGGAACTGAAG |
| 67 | Tri-functional | | | MDMRVPAQLLGLLQLWLSGARCDIV |
| | Engager 301 | | | MTQSPSSLSASVGDRVTITCKASQS |
| | (including signal | | | VTNDVAWYQQKPGKAPKLLIYYASN |
| | sequence and C- | | | RYTGVPDRFSGSGYGTDFTLTISSL |
| | terminal GGS | | | QPEDFATYFCQQDYRSPWTFGGGTK |
| | linker-His tag) | | | VEIKGGGGSGGGGSGGGGSGGGGSQ |
| | | | | VQLQESGPALVKPTQTLTLTCTVSG |
| | | | | FSLSDYGVNWIRQPPGKALEWLGII |
| | | | | WGDGRTDYNSALKSRLSISKDNSKN |
| | | | | QVVLRMTNVDPVDTATYFCARAPGN |
| | | | | RAMEYWGQGTVVTVSSASTSGGGGS |
| | | | | GGGGSGGGGSGGGGSGITCPPPMSV |
| | | | | EHADIWVKSYSLYSRERYICNSGFK |
| | | | | RKAGTSSLTECVLNKATNVAHWTTP |
| | | | | SLKCIRDPALVHQRPAPPGGGSGGG |
| | | | | GSGGGSGGGGSLQNWVNVISDLKKI |
| | | | | EDLIQSMHIDATLYTESDVHPSCKV |
| | | | | TAMKCFLLELQVISLESGDASIHDT |
| | | | | VENLIILANNSLSSNGNVTESGCKE |
| | | | | CEELEEKNIKEFLQSFVHIVQMFIN |
| | | | | TSGSTSGSGKPGSGEGSTKGQVQLQ |
| | | | | QSGAELARPGASVKMSCKTSGYTFT |
| | | | | RYTMHWVKQRPGQGLEWIGYINPSR |
| | | | | GYTNYNQKFKDKATLTTDKSSSTAY |
| | | | | MQLSSLTSEDSAVYYCARYYDDHYS |
| | | | | LDYWGQGTTLTVSSGGGGSGGGGSG |
| | | | | GGGSGGGGSQIVLTQSPAIMSASPG |
| | | | | EKVTMTCRASSSVSYMNWYQQKSGT |
| | | | | SPKRWIYDTSKVASGVPYRFSGSGS |
| | | | | GTSYSLTISSMEAEDAATYYCQQWS |
| | | | | SNPLTFGAGTKLELKGGSHHHHHH* |
| 68 | Tri-functional | | | ATGGACATGAGGGTCCCTGCTCAGC |
| | Engager 301 | | | TCCTGGGGCTCCTGCAGCTCTGGCT |
| | (including signal | | | CTCAGGTGCCAGATGTGACATCGTG |
| | sequence and | | | ATGACCCAGTCTCCATCCTCCCTGT |
| | His tag) | | | CTGCATCTGTAGGAGACAGAGTCAC |
| | (nucleotide | | | CATCACTTGCAAGGCCAGTCAGAGT |
| | sequence) | | | GTGACTAATGATGTAGCTTGGTATC |
| | | | | AGCAGAAACCAGGGAAAGCCCCTAA |
| | | | | GCTCCTGATCTACTATGCATCCAAT |
| | | | | CGCTACACTGGGGTCCCAGATAGGT |
| | | | | TCAGTGGAAGTGGATATGGGACAGA |
| | | | | TTTTACTTTGACCATCAGCAGCCTG |
| | | | | CAGCCTGAAGATTTTGCAACATATT |
| | | | | TCTGTCAGCAGGATTATAGGTCTCC |
| | | | | GTGGACGTTTGGCGGCGGTACCAAA |
| | | | | GTGGAGATCAAAGGCGGCGGCGGAT |
| | | | | CTGGTGGCGGAGGGTCAGGGGGAGG |
| | | | | CGGTAGTGGTGGCGGAGGGAGCCAG |
| | | | | GTCCAGTTGCAGGAGTCTGGTCCTG |
| | | | | CGCTGGTGAAACCCACACAGACCCT |
| | | | | CACACTGACCTGCACCGTGTCTGGG |
| | | | | TTCTCACTCAGCGACTATGGTGTAA |
| | | | | ACTGGATCCGTCAGCCCCCAGGGAA |
| | | | | GGCCCTGGAGTGGCTTGGAATAATA |
| | | | | TGGGGTGATGGAAGGACAGACTATA |
| | | | | ATTCAGCTCTCAAATCCAGGCTCAG |
| | | | | CATCTCCAAGGACAACTCCAAAAAC |
| | | | | CAGGTGGTCCTTAGAATGACCAACG |
| | | | | TGGACCCTGTGGACACAGCCACGTA |
| | | | | TTTCTGTGCAAGAGCCCCCGGTAAT |
| | | | | AGGGCTATGGAGTACTGGGGCCAGG |
| | | | | GAACCGTGGTCACCGTCTCCTCAGC |
| | | | | CTCCACCTCCGGCGGCGGAGGAAGC |
| | | | | GGTGGGGGCGGTTCTGGAGGCGGTG |
| | | | | GTAGTGGCGGAGGAGGCAGTGGAAT |
| | | | | CACGTGCCCTCCCCCCATGTCCGTG |
| | | | | GAACACGCAGACATCTGGGTCAAGA |
| | | | | GCTACAGCTTGTACTCCAGGGAGCG |
| | | | | GTACATTTGTAACTCTGGTTTCAAG |
| | | | | CGTAAAGCCGGCACGTCCAGCCTGA |
| | | | | CGGAGTGCGTGTTGAACAAGGCCAC |
| | | | | GAATGTCGCCCACTGGACAACCCCC |
| | | | | AGTCTCAAATGCATTAGAGACCCTG |
| | | | | CCCTGGTTCACCAAAGGCCAGCGCC |
| | | | | ACCCGGCGGAGGATCAGGCGGGGGC |
| | | | | GGTAGTGGTGGCGGTTCTGGTGGTG |
| | | | | GAGGTTCATTGCAAAATTGGGTAAA |
| | | | | TGTGATCTCTGACCTGAAGAAGATT |
| | | | | GAGGATCTCATCCAATCTATGCATA |
| | | | | TCGACGCCACCCTCTATACCGAAAG |
| | | | | TGATGTCCACCCCAGCTGCAAGGTA |
| | | | | ACAGCAATGAAATGTTTTCTGCTCG |
| | | | | AACTTCAAGTGATATCCTTGGAATC |
| | | | | AGGCGACGCATCAATCCATGATACA |
| | | | | GTCGAAAATCTCATCATTCTGGCAA |
| | | | | ACAATTCCCTCTCCAGCAATGGCAA |
| | | | | CGTCACTGAGTCAGGATGCAAAGAG |
| | | | | TGCGAAGAGCTCGAAGAAAAGAATA |
| | | | | TCAAGGAGTTCCTGCAAAGCTTTGT |
| | | | | TCATATCGTCCAAATGTTCATTAAT |
| | | | | ACATCCGGGAGTACGTCAGGATCTG |
| | | | | GGAAGCCAGGCTCAGGAGAGGGCTC |
| | | | | TACAAAGGGACAGGTGCAGCTGCAG |
| | | | | CAGTCTGGCGCTGAGCTGGCTAGAC |
| | | | | CTGGCGCCTCCGTGAAGATGTCCTG |
| | | | | CAAGACCTCCGGCTACACCTTCACC |
| | | | | CGGTACACCATGCACTGGGTCAAGC |
| | | | | AGAGGCCTGGACAGGGCCTGGAATG |
| | | | | GATCGGCTACATCAACCCCTCCCGG |
| | | | | GGCTACACCAACTACAACCAGAAGT |
| | | | | TCAAGGACAAGGCCACCCTGACAAC |
| | | | | CGACAAGTCCTCCTCCACCGCCTAC |
| | | | | ATGCAGCTGTCCTCCCTGACCTCCG |
| | | | | AGGACTCCGCCGTGTACTACTGCGC |
| | | | | CCGGTACTACGACGACCACTACTCC |
| | | | | CTGGACTACTGGGGCCAGGGCACCA |
| | | | | CACTGACAGTGTCTAGCGGAGGCGG |
| | | | | AGGATCTGGTGGTGGCGGATCTGGC |
| | | | | GGCGGTGGAAGTGGCGGAGGTGGTA |
| | | | | GCCAGATCGTGCTGACCCAGTCTCC |
| | | | | CGCCATCATGTCTGCTAGCCCTGGC |
| | | | | GAGAAAGTGACAATGACCTGCCGGG |
| | | | | CCTCCTCCTCCGTGTCCTACATGAA |
| | | | | CTGGTATCAGCAGAAGTCCGGCACC |
| | | | | TCCCCCAAGCGGTGGATCTACGACA |
| | | | | CCTCCAAGGTGGCCTCTGGCGTGCC |
| | | | | CTACAGATTCTCCGGCTCTGGCTCT |
| | | | | GGCACCTCCTACAGCCTGACCATCT |
| | | | | CCAGCATGGAAGCCGAGGATGCCGC |
| | | | | CACCTACTACTGCCAGCAGTGGTCC |
| | | | | TCCAACCCCCTGACCTTTGGCGCTG |
| | | | | GCACCAAGCTGGAACTGAAGGGCGG |
| | | | | CTCTCATCACCATCATCACCATTGA |
| 69 | Linker | | | TCCGGCGGCGGAGGAAGCGGTGGGG |
| | (SG₄)₄SG | | | GCGGTTCTGGAGGCGGTGGTAGTGG |
| | (nucleotide | | | CGGAGGAGGCAGTGGA |
| | sequence) | | | |
| 70 | Sushi domain (aa | | | ATCACGTGCCCTCCCCCCATGTCCG |
| | 31-107) | | | TGGAACACGCAGACATCTGGGTCAA |
| | (nucleotide | | | GAGCTACAGCTTGTACTCCAGGGAG |
| | sequence) | | | CGGTACATTTGTAACTCTGGTTTCA |
| | | | | AGCGTAAAGCCGGCACGTCCAGCCT |
| | | | | GACGGAGTGCGTGTTGAACAAGGCC |
| | | | | ACGAATGTCGCCCACTGGACAACCC |
| | | | | CCAGTCTCAAATGCATTAGAGACCC |
| | | | | TGCCCTGGTTCACCAAAGGCCAGCG |
| | | | | CCACCC |
| 71 | Linker | | | GGCGGAGGATCAGGCGGGGGCGGTA |
| | (GGGSGGGGS | | | GTGGTGGCGGTTCTGGTGGTGGAGG |
| | GGGSGGGGSLQ) | | | TTCATTGCAA |
| | (nucleotide | | | |
| | sequence) | | | |
| 72 | Human IL-15 | | | AATTGGGTAAATGTGATCTCTGACC |
| | (nucleotide | | | TGAAGAAGATTGAGGATCTCATCCA |
| | sequence) | | | ATCTATGCATATCGACGCCACCCTC |
| | | | | TATACCGAAAGTGATGTCCACCCCA |
| | | | | GCTGCAAGGTAACAGCAATGAAATG |
| | | | | TTTTCTGCTCGAACTTCAAGTGATA |
| | | | | TCCTTGGAATCAGGCGACGCATCAA |
| | | | | TCCATGATACAGTCGAAAATCTCAT |
| | | | | CATTCTGGCAAACAATTCCCTCTCC |
| | | | | AGCAATGGCAACGTCACTGAGTCAG |
| | | | | GATGCAAAGAGTGCGAAGAGCTCGA |
| | | | | AGAAAAGAATATCAAGGAGTTCCTG |
| | | | | CAAAGCTTTGTTCATATCGTCCAAA |
| | | | | TGTTCATTAATACATCC |
| 73 | Whitlow Linker | | | GGGAGTACGTCAGGATCTGGGAAGC |
| | (nucleotide | | | CAGGCTCAGGAGAGGGCTCTACAAA |
| | sequence) | | | GGGA |
| 74 | IL-15 mutein | | | NWVNVISDLKKIEDLIQSMHIDATL |
| | (N72A) | | | YTESDVHPSCKVTAMKCFLLELQVI |
| | | | | SLESGDASIHDTVENLIILANASLS |
| | | | | SNGNVTESGCKECEELEEKNIKEFL |
| | | | | QSFVHIVQMFINTS |
| 75 | IL-15 mutein | | | NWVNVISDLKKIEDLIQSMHIDATL |
| | (N72E) | | | YTESDVHPSCKVTAMKCFLLELQVI |
| | | | | SLESGDASIHDTVENLIILANESLS |
| | | | | SNGNVTESGCKECEELEEKNIKEFL |
| | | | | QSFVHIVQMFINTS |
| 76 | IL-15 mutein | | | NWVNVISDLKKIEDLIQSMHIDATL |
| | (N72S) | | | YTESDVHPSCKVTAMKCFLLELQVI |
| | | | | SLESGDASIHDTVENLIILANSSLS |
| | | | | SNGNVTESGCKECEELEEKNIKEFL |
| | | | | QSFVHIVQMFINTS |
| 77 | IL-15 mutein | | | NWVNVISDLKKIEDLIQSMHIDATL |
| | (N72Y) | | | YTESDVHPSCKVTAMKCFLLELQVI |
| | | | | SLESGDASIHDTVENLIILANYSLS |
| | | | | SNGNVTESGCKECEELEEKNIKEFL |
| | | | | QSFVHIVQMFINTS |
| 78 | IL-15 mutein | | | NWVNVISDLKKIEDLIQSMHIDATL |
| | (N72P) | | | YTESDVHPSCKVTAMKCFLLELQVI |
| | | | | SLESGDASIHDTVENLIILANPSLS |
| | | | | SNGNVTESGCKECEELEEKNIKEFL |
| | | | | QSFVHIVQMFINTS |
| 79 | BiTE 198 | | | MDMRVPAQLLGLLQLWLSGARCDIV |
| | (includes signal | | | MTQSPSSLSASVGDRVTITCKASQS |
| | sequence and C- | | | VTNDVAWYQQKPGKAPKLLIYYASN |
| | terminal GGS- | | | RYTGVPDRFSGSGYGTDFTLTISSL |
| | His tag) | | | QPEDFATYFCQQDYRSPWTFGGGTK |
| | | | | VEIKGGGGSGGGGSGGGGSGGGGSQ |
| | | | | VQLQESGPALVKPTQTLTLTCTVSG |
| | | | | FSLSDYGVNWIRQPPGKALEWLGII |
| | | | | WGDGRTDYNSALKSRLSISKDNSKN |
| | | | | QVVLRMTNVDPVDTATYFCARAPGN |
| | | | | RAMEYWGQGTVVTVSSASTGGGGSG |
| | | | | GGGSGGGGSLAEAKVLANRELDKYG |
| | | | | VSDFYKRLINKAKTVEGVEALKLHI |
| | | | | LAALPGGGGSGGGGSGGGGSQVQLQ |
| | | | | QSGAELARPGASVKMSCKTSGYTFT |
| | | | | RYTMHWVKQRPGQGLEWIGYINPSR |
| | | | | GYTNYNQKFKDKATLTTDKSSSTAY |
| | | | | MQLSSLTSEDSAVYYCARYYDDHYS |
| | | | | LDYWGQGTTLTVSSGGGGSGGGGSG |
| | | | | GGGSGGGGSQIVLTQSPAIMSASPG |
| | | | | EKVTMTCRASSSVSYMNWYQQKSGT |
| | | | | SPKRWIYDTSKVASGVPYRFSGSGS |
| | | | | GTSYSLTISSMEAEDAATYYCQQWS |
| | | | | SNPLTFGAGTKLELKGGSHHHHHH |
| 80 | BiTE 198 | | | ATGGACATGAGGGTCCCTGCTCAGC |
| | (includes signal | | | TCCTGGGGCTCCTGCAGCTCTGGCT |
| | sequence and | | | CTCAGGTGCCAGATGTGACATCGTG |
| | His tag) | | | ATGACCCAGTCTCCATCCTCCCTGT |
| | (nucleotide | | | CTGCATCTGTAGGAGACAGAGTCAC |
| | sequence) | | | CATCACTTGCAAGGCCAGTCAGAGT |
| | | | | GTGACTAATGATGTAGCTTGGTATC |
| | | | | AGCAGAAACCAGGGAAAGCCCCTAA |
| | | | | GCTCCTGATCTACTATGCATCCAAT |
| | | | | CGCTACACTGGGGTCCCAGATAGGT |
| | | | | TCAGTGGAAGTGGATATGGGACAGA |
| | | | | TTTTACTTTGACCATCAGCAGCCTG |
| | | | | CAGCCTGAAGATTTTGCAACATATT |
| | | | | TCTGTCAGCAGGATTATAGGTCTCC |
| | | | | GTGGACGTTTGGCGGCGGTACCAAA |
| | | | | GTGGAGATCAAAGGCGGCGGCGGAT |
| | | | | CTGGTGGCGGAGGGTCAGGGGGAGG |
| | | | | CGGTAGTGGTGGCGGAGGGAGCCAG |
| | | | | GTCCAGTTGCAGGAGTCTGGTCCTG |
| | | | | CGCTGGTGAAACCCACACAGACCCT |
| | | | | CACACTGACCTGCACCGTGTCTGGG |
| | | | | TTCTCACTCAGCGACTATGGTGTAA |
| | | | | ACTGGATCCGTCAGCCCCCAGGGAA |
| | | | | GGCCCTGGAGTGGCTTGGAATAATA |
| | | | | TGGGGTGATGGAAGGACAGACTATA |
| | | | | ATTCAGCTCTCAAATCCAGGCTCAG |
| | | | | CATCTCCAAGGACAACTCCAAAAAC |
| | | | | CAGGTGGTCCTTAGAATGACCAACG |
| | | | | TGGACCCTGTGGACACAGCCACGTA |
| | | | | TTTCTGTGCAAGAGCCCCCGGTAAT |
| | | | | AGGGCTATGGAGTACTGGGGCCAGG |
| | | | | GAACCGTGGTCACCGTCTCCTCAGC |
| | | | | CTCCACCGGAGGAGGCGGCTCTGGA |
| | | | | GGTGGCGGCAGTGGTGGAGGCGGGT |
| | | | | CTCTGGCCGAGGCCAAGGTGCTGGC |
| | | | | CAACAGAGAGCTGGACAAGTACGGC |
| | | | | GTGAGCGACTTCTACAAGAGACTGA |
| | | | | TCAACAAGGCCAAGACCGTGGAGGG |
| | | | | CGTGGAGGCCCTGAAGCTGCACATC |
| | | | | CTGGCCGCCCTGCCCGGCGGTGGCG |
| | | | | GATCTGGAGGTGGTGGGAGCGGCGG |
| | | | | AGGCGGATCTCAGGTGCAGCTGCAG |
| | | | | CAGTCTGGCGCTGAGCTGGCTAGAC |
| | | | | CTGGCGCCTCCGTGAAGATGTCCTG |
| | | | | CAAGACCTCCGGCTACACCTTCACC |
| | | | | CGGTACACCATGCACTGGGTCAAGC |
| | | | | AGAGGCCTGGACAGGGCCTGGAATG |
| | | | | GATCGGCTACATCAACCCCTCCCGG |
| | | | | GGCTACACCAACTACAACCAGAAGT |
| | | | | TCAAGGACAAGGCCACCCTGACAAC |
| | | | | CGACAAGTCCTCCTCCACCGCCTAC |
| | | | | ATGCAGCTGTCCTCCCTGACCTCCG |
| | | | | AGGACTCCGCCGTGTACTACTGCGC |
| | | | | CCGGTACTACGACGACCACTACTCC |
| | | | | CTGGACTACTGGGGCCAGGGCACCA |
| | | | | CACTGACAGTGTCTAGCGGAGGCGG |
| | | | | AGGATCTGGTGGTGGCGGATCTGGC |
| | | | | GGCGGTGGAAGTGGCGGAGGTGGTA |
| | | | | GCCAGATCGTGCTGACCCAGTCTCC |
| | | | | CGCCATCATGTCTGCTAGCCCTGGC |
| | | | | GAGAAAGTGACAATGACCTGCCGGG |
| | | | | CCTCCTCCTCCGTGTCCTACATGAA |
| | | | | CTGGTATCAGCAGAAGTCCGGCACC |
| | | | | TCCCCCAAGCGGTGGATCTACGACA |
| | | | | CCTCCAAGGTGGCCTCTGGCGTGCC |
| | | | | CTACAGATTCTCCGGCTCTGGCTCT |
| | | | | GGCACCTCCTACAGCCTGACCATCT |
| | | | | CCAGCATGGAAGCCGAGGATGCCGC |
| | | | | CACCTACTACTGCCAGCAGTGGTCC |
| | | | | TCCAACCCCCTGACCTTTGGCGCTG |
| | | | | GCACCAAGCTGGAACTGAAGGGCGG |
| | | | | CTCTCACCACCACCATCACCAC |
| 81 | (G₄S)₃ Linker | | | GGCGGTGGCGGATCTGGAGGTGGTG |
| | | | | GGAGCGGCGGAGGCGGATCT |
| 82 | Albumin binding | | | LAEAKVLANRELDKYGVSDFYKRLI |
| | domain | | | NKAKTVEGVEALKLHILAALP |
| 83 | Albumin binding | | | CTGGCCGAGGCCAAGGTGCTGGCCA |
| | domain | | | ACAGAGAGCTGGACAAGTACGGCGT |
| | (nucleotide | | | GAGCGACTTCTACAAGAGACTGATC |
| | sequence) | | | AACAAGGCCAAGACCGTGGAGGGCG |
| | | | | TGGAGGCCCTGAAGCTGCACATCCT |
| | | | | GGCCGCCCTGCCC |
| 84 | Linker (Gly₈) | | | GGGGGGGG |
| 85 | Linker (Gly₆) | | | GGGGGG |
| 86 | Linker | | | GGGGS |
| 87 | Linker | | | KESGSVSSEQLAQFRSLD |
| 88 | Linker | | | EGKSSGSGSESKST |
| 89 | Linker | | | GSAGSAAGSGEF |
| 90 | Linker (G₄S)₄ | | | GGAGGCGGAGGATCTGGTGGTGGCG |
| | (nucleotide | | | GATCTGGCGGCGGTGGAAGTGGCGG |
| | sequence) | | | AGGTGGTAGC |
| 91 | BiTE 198 | | | DIVMTQSPSSLSASVGDRVTITCKA |
| | (without signal | | | SQSVTNDVAWYQQKPGKAPKLLIYY |
| | peptide or GGS- | | | ASNRYTGVPDRFSGSGYGTDFTLTI |
| | His tag) | | | SSLQPEDFATYFCQQDYRSPWTFGG |
| | | | | GTKVEIKGGGGSGGGGSGGGGSGGG |
| | | | | GSQVQLQESGPALVKPTQTLTLTCT |
| | | | | VSGFSLSDYGVNWIRQPPGKALEWL |
| | | | | GIIWGDGRTDYNSALKSRLSISKDN |
| | | | | SKNQVVLRMTNVDPVDTATYFCARA |
| | | | | PGNRAMEYWGQGTVVTVSSASTGGG |
| | | | | GSGGGGSGGGGSLAEAKVLANRELD |
| | | | | KYGVSDFYKRLINKAKTVEGVEALK |
| | | | | LHILAALPGGGGSGGGGSGGGGSQV |
| | | | | QLQQSGAELARPGASVKMSCKTSGY |
| | | | | TFTRYTMHWVKQRPGQGLEWIGYIN |
| | | | | PSRGYTNYNQKFKDKATLTTDKSSS |
| | | | | TAYMQLSSLTSEDSAVYYCARYYDD |
| | | | | HYSLDYWGQGTTLTVSSGGGGSGGG |
| | | | | GSGGGGSGGGGSQIVLTQSPAIMSA |
| | | | | SPGEKVTMTCRASSSVSYMNWYQQK |
| | | | | SGTSPKRWIYDTSKVASGVPYRFSG |
| | | | | SGSGTSYSLTISSMEAEDAATYYCQ |
| | | | | QWSSNPLTFGAGTKLELK |
| 92 | Linker | | | GGGGSGGGGSGGSA |
| 93 | Linker | | | SGGGGSGGGG |
| 94 | Linker | | | SGSGGGGSLQ |
| 95 | Tri-functional | | | GACCGATCCAGCCTCCGGACTCTAG |
| | engager (IL-12) | | | AGGATCGAACCCTTGCCACCATGGA |
| | (nucleotide | | | CATGAGGGTCCCTGCTCAGCTCCTG |
| | sequence) | | | GGGCTCCTGCAGCTCTGGCTCTCAG |
| | | | | GTGCCAGATGTGACATCGTGATGAC |
| | | | | CCAGTCTCCATCCTCCCTGTCTGCA |
| | | | | TCTGTAGGAGACAGAGTCACCATCA |
| | | | | CTTGCAAGGCCAGTCAGAGTGTGAC |
| | | | | TAATGATGTAGCTTGGTATCAGCAG |
| | | | | AAACCAGGGAAAGCCCCTAAGCTCC |
| | | | | TGATCTACTATGCATCCAATCGCTA |
| | | | | CACTGGGGTCCCAGATAGGTTCAGT |
| | | | | GGAAGTGGATATGGGACAGATTTTA |
| | | | | CTTTGACCATCAGCAGCCTGCAGCC |
| | | | | TGAAGATTTTGCAACATATTTCTGT |
| | | | | CAGCAGGATTATAGGTCTCCGTGGA |
| | | | | CGTTTGGCGGCGGTACCAAAGTGGA |
| | | | | GATCAAAGGCGGCGGCGGATCTGGT |
| | | | | GGCGGAGGGTCAGGGGGAGGCGGTA |
| | | | | GTGGCGGAGGGAGCCAGGTCCAGTT |
| | | | | GCAGGAGTCTGGTCCTGCGCTGGTG |
| | | | | AAACCCACACAGACCCTCACACTGA |
| | | | | CCTGCACCGTGTCTGGGTTCTCACT |
| | | | | CAGCGACTATGGTGTAAACTGGATC |
| | | | | CGTCAGCCCCCAGGGAAGGCCCTGG |
| | | | | AGTGGCTTGGAATAATATGGGGTGA |
| | | | | TGGAAGGACAGACTATAATTCAGCT |
| | | | | CTCAAATCCAGGCTCAGCATCTCCA |
| | | | | AGGACAACTCCAAAAACCAGGTGGT |
| | | | | CCTTAGAATGACCAACGTGGACCCT |
| | | | | GTGGACACAGCCACGTATTTCTGTG |
| | | | | CAAGAGCCCCCGGTAATAGGGCTAT |
| | | | | GGAGTACTGGGGCCAGGGAACCGTG |
| | | | | GTCACCGTCTCCTCAGCCTCCACCT |
| | | | | CCGGCGGCGGAGGAAGCGGTGGGGG |
| | | | | CGGTTCTGGAGGCGGTGGTAGTGGC |
| | | | | GGAGGAGGCAGTGGAATCTGGGAGC |
| | | | | TTAAAAAGGATGTATACGTAGTCGA |
| | | | | GCTTGATTGGTACCCCGACGCGCCT |
| | | | | GGGGAAATGGTGGTGCTCACATGTG |
| | | | | ATACCCCGGAAGAAGACGGGATAAC |
| | | | | TTGGACTCTTGACCAAAGCTCCGAG |
| | | | | GTGTTGGGGAGTGGCAAGACGCTCA |
| | | | | CAATCCAAGTCAAGGAATTCGGGGA |
| | | | | CGCCGGTCAATACACATGTCACAAG |
| | | | | GGTGGCGAGGTGCTGAGCCACTCTC |
| | | | | TTCTTCTTTTGCATAAAAAGGAGGA |
| | | | | CGGAATATGGTCCACAGACATCCTG |
| | | | | AAGGACCAGAAAGAACCGAAAAATA |
| | | | | AAACATTCCTCAGGTGCGAGGCCAA |
| | | | | GAACTATTCTGGCAGATTTACGTGT |
| | | | | TGGTGGCTTACCACTATTTCAACCG |
| | | | | ACCTTACTTTCAGTGTGAAGAGTAG |
| | | | | CAGAGGAAGTTCAGACCCTCAAGGT |
| | | | | GTCACCTGCGGTGCCGCCACGCTGT |
| | | | | CAGCGGAAAGAGTTAGAGGGGACAA |
| | | | | CAAGGAGTATGAATACAGCGTTGAG |
| | | | | TGTCAAGAGGATAGCGCGTGTCCCG |
| | | | | CCGCTGAGGAGTCCCTTCCGATAGA |
| | | | | GGTGATGGTCGATGCAGTGCATAAA |
| | | | | CTCAAATACGAGAACTACACGAGTT |
| | | | | CTTTCTTCATTAGAGATATCATCAA |
| | | | | ACCTGATCCTCCGAAAAACCTCCAA |
| | | | | CTCAAGCCGCTCAAAAACAGTCGCC |
| | | | | AGGTTGAAGTCAGTTGGGAGTACCC |
| | | | | TGATACTTGGAGCACCCCACACTCC |
| | | | | TATTTCTCCCTGACGTTTTGTGTAC |
| | | | | AGGTTCAGGGCAAAAGTAAAAGGGA |
| | | | | AAAAAAAGACCGCGTGTTCACAGAT |
| | | | | AAGACCTCAGCAACTGTCATTTGTA |
| | | | | GAAAAAACGCATCTATTAGCGTCCG |
| | | | | GGCACAGGACCGGTATTACTCATCT |
| | | | | TCCTGGTCAGAGTGGGCGAGTGTGC |
| | | | | CTTGCTCTGGTGGAGGGGGATCTGG |
| | | | | AGGAGGCGGTTCAGGAGGAGGGGGG |
| | | | | TCCAGGAATCTGCCTGTCGCGACGC |
| | | | | CCGACCCGGGTATGTTTCCGTGCTT |
| | | | | GCACCACTCACAAAACCTTCTGCGC |
| | | | | GCCGTCAGTAATATGTTGCAAAAGG |
| | | | | CTAGGCAAACCCTTGAATTTTATCC |
| | | | | GTGCACCTCCGAAGAAATAGACCAT |
| | | | | GAGGACATTACCAAGGACAAGACGT |
| | | | | CTACAGTGGAGGCCTGCTTGCCCCT |
| | | | | TGAGCTCACAAAGAATGAGAGCTGC |
| | | | | TTGAATTCACGCGAAACATCTTTTA |
| | | | | TCACGAACGGGAGTTGCCTGGCCTC |
| | | | | CCGGAAGACGTCTTTTATGATGGCT |
| | | | | CTGTGTTTGTCATCAATTTACGAGG |
| | | | | ATTTGAAGATGTACCAAGTGGAATT |
| | | | | CAAGACAATGAATGCGAAACTGCTG |
| | | | | ATGGACCCGAAGCGCCAAATCTTCC |
| | | | | TCGATCAAAATATGCTCGCTGTCAT |
| | | | | CGACGAACTGATGCAGGCGCTCAAC |
| | | | | TTCAATTCCGAGACGGTGCCGCAGA |
| | | | | AAAGTAGCCTGGAAGAGCCGGATTT |
| | | | | CTATAAAACAAAAATCAAACTGTGC |
| | | | | ATCTTGCTGCACGCATTTAGGATTC |
| | | | | GAGCTGTAACTATCGATCGCGTAAT |
| | | | | GTCTTATCTGAACGCTAGTGGGAGT |
| | | | | ACGTCAGGATCTGGGAAGCCAGGCT |
| | | | | CAGGAGAGGGCTCTACAAAGGGACA |
| | | | | GGTGCAGCTGCAGCAGTCTGGCGCT |
| | | | | GAGCTGGCTAGACCTGGCGCCTCCG |
| | | | | TGAAGATGTCCTGCAAGACCTCCGG |
| | | | | CTACACCTTCACCCGGTACACCATG |
| | | | | CACTGGGTCAAGCAGAGGCCTGGAC |
| | | | | AGGGCCTGGAATGGATCGGCTACAT |
| | | | | CAACCCCTCCCGGGGCTACACCAAC |
| | | | | TACAACCAGAAGTTCAAGGACAAGG |
| | | | | CCACCCTGACAACCGACAAGTCCTC |
| | | | | CTCCACCGCCTACATGCAGCTGTCC |
| | | | | TCCCTGACCTCCGAGGACTCCGCCG |
| | | | | TGTACTACTGCGCCCGGTACTACGA |
| | | | | CGACCACTACTCCCTGGACTACTGG |
| | | | | GGCCAGGGCACCACACTGACAGTGT |
| | | | | CTAGCGGAGGCGGAGGATCTGGTGG |
| | | | | TGGCGGATCTGGCGGCGGTGGAAGT |
| | | | | GGCGGAGGTGGTAGCCAGATCGTGC |
| | | | | TGACCCAGTCTCCCGCCATCATGTC |
| | | | | TGCTAGCCCTGGCGAGAAAGTGACA |
| | | | | ATGACCTGCCGGGCCTCCTCCTCCG |
| | | | | TGTCCTACATGAACTGGTATCAGCA |
| | | | | GAAGTCCGGCACCTCCCCCAAGCGG |
| | | | | TGGATCTACGACACCTCCAAGGTGG |
| | | | | CCTCTGGCGTGCCCTACAGATTCTC |
| | | | | CGGCTCTGGCTCTGGCACCTCCTAC |
| | | | | AGCCTGACCATCTCCAGCATGGAAG |
| | | | | CCGAGGATGCCGCCACCTACTACTG |
| | | | | CCAGCAGTGGTCCTCCAACCCCCTG |
| | | | | ACCTTTGGCGCTGGCACCAAGCTGG |
| | | | | AACTGAAGGGCGGCTCTCATCACCA |
| | | | | TCATCACCATTGAAAGGGTTCGATC |
| | | | | CCTACCGGTTAGTAATGAGTTTAAA |
| | | | | CGGG |
| 96 | Tri-functional | | | DIVMTQSPSSLSASVGDRVTITCKA |
| | engager (IL-12) | | | SQSVTNDVAWYQQKPGKAPKLLIYY |
| | | | | ASNRYTGVPDRFSGSGYGTDFTLTI |
| | | | | SSLQPEDFATYFCQQDYRSPWTFGG |
| | | | | GTKVEIKGGGGSGGGGSGGGGSGGG |
| | | | | SQVQLQESGPALVKPTQTLTLTCTV |
| | | | | SGFSLSDYGVNWIRQPPGKALEWLG |
| | | | | IIWGDGRTDYNSALKSRLSISKDNS |
| | | | | KNQVVLRMTNVDPVDTATYFCARAP |
| | | | | GNRAMEYWGQGTVVTVSSASTSGGG |
| | | | | GSGGGGSGGGGSGGGGSGIWELKKD |
| | | | | VYVVELDWYPDAPGEMVVLTCDTPE |
| | | | | EDGITWTLDQSSEVLGSGKTLTIQV |
| | | | | KEFGDAGQYTCHKGGEVLSHSLLLL |
| | | | | HKKEDGIWSTDILKDQKEPKNKTFL |
| | | | | RCEAKNYSGRFTCWWLTTISTDLTF |
| | | | | SVKSSRGSSDPQGVTCGAATLSAER |
| | | | | VRGDNKEYEYSVECQEDSACPAAEE |
| | | | | SLPIEVMVDAVHKLKYENYTSSFFI |
| | | | | RDIIKPDPPKNLQLKPLKNSRQVEV |
| | | | | SWEYPDTWSTPHSYFSLTFCVQVQG |
| | | | | KSKREKKDRVFTDKTSATVICRKNA |
| | | | | SISVRAQDRYYSSSWSEWASVPCSG |
| | | | | GGGSGGGGSGGGGSRNLPVATPDPG |
| | | | | MFPCLHHSQNLLRAVSNMLQKARQT |
| | | | | LEFYPCTSEEIDHEDITKDKTSTVE |
| | | | | ACLPLELTKNESCLNSRETSFITNG |
| | | | | SCLASRKTSFMMALCLSSIYEDLKM |
| | | | | YQVEFKTMNAKLLMDPKRQIFLDQN |
| | | | | MLAVIDELMQALNFNSETVPQKSSL |
| | | | | EEPDFYKTKIKLCILLHAFRIRAVT |
| | | | | IDRVMSYLNASGSTSGSGKPGSGEG |
| | | | | STKGQVQLQQSGAELARPGASVKMS |
| | | | | CKTSGYTFTRYTMHWVKQRPGQGLE |
| | | | | WIGYINPSRGYTNYNQKFKDKATLT |
| | | | | TDKSSSTAYMQLSSLTSEDSAVYYC |
| | | | | ARYYDDHYSLDYWGQGTTLTVSSGG |
| | | | | GGSGGGGSGGGGSGGGGSQIVLTQS |
| | | | | PAIMSASPGEKVTMTCRASSSVSYM |
| | | | | NWYQQKSGTSPKRWIYDTSKVASGV |
| | | | | PYRFSGSGSGTSYSLTISSMEAEDA |
| | | | | ATYYCQQWSSNPLTFGAGTKLELK |
| 97 | IL-12 p40 | | | IWELKKDVYVVELDWYPDAPGEMVV |
| | | | | LTCDTPEEDGITWTLDQSSEVLGSG |
| | | | | KTLTIQVKEFGDAGQYTCHKGGEVL |
| | | | | SHSLLLLHKKEDGIWSTDILKDQKE |
| | | | | PKNKTFLRCEAKNYSGRFTCWWLTT |
| | | | | ISTDLTFSVKSSRGSSDPQGVTCGA |
| | | | | ATLSAERVRGDNKEYEYSVECQEDS |
| | | | | ACPAAEESLPIEVMVDAVHKLKYEN |
| | | | | YTSSFFIRDIIKPDPPKNLQLKPLK |
| | | | | NSRQVEVSWEYPDTWSTPHSYFSLT |
| | | | | FCVQVQGKSKREKKDRVFTDKTSAT |
| | | | | VICRKNASISVRAQDRYYSSSWSEW |
| | | | | ASVPCS |
| 98 | IL-12 p35 | | | RNLPVATPDPGMFPCLHHSQNLLRA |
| | | | | VSNMLQKARQTLEFYPCTSEEIDHE |
| | | | | DITKDKTSTVEACLPLELTKNESCL |
| | | | | NSRETSFITNGSCLASRKTSFMMAL |
| | | | | CLSSIYEDLKMYQVEFKTMNAKLLM |
| | | | | DPKRQIFLDQNMLAVIDELMQALNF |
| | | | | NSETVPQKSSLEEPDFYKTKIKLCI |
| | | | | LLHAFRIRAVTIDRVMSYLNAS |
| 99 | Linker | | | GGGGSGGGGSGGGS |
| 100 | Human IL-12 | | | MLQQQLVLSCFSLVLLASPLAAIWE |
| | p40 | | | LEKDVYVVELDWYPDAPGEMVVLTC |
| | | | | DTPEEEGITWTSDQSSEVLGSGKTL |
| | | | | TIQVKEFGDAGQYTCHKGDTILSQS |
| | | | | LLLLHKKEDGIWSTDILKDQKEPKN |
| | | | | KTFLKCKAKNYSGHFTCWWLTAIST |
| | | | | DLKFSVKSSRGSSDPRGVTCGPATL |
| | | | | SAERVRVDNREYKKYTVECQEGSSC |
| | | | | PAAEESLPIEVLVDAVHKLKYENYT |
| | | | | SSFFIRDIIKPDPPKNLQLKPLKNS |
| | | | | RHVEVSWEYPDTWSTPHSYFSLTFS |
| | | | | LQVQGKNKREKKDRLFMDQTSAKVT |
| | | | | CHKDAVIRVQARDRYYSSSWSEWAS |
| | | | | VSCS |
| 101 | Human IL-12 | | | MWPPGSASQPPPSPAAATGLHPAAR |
| | p35 | | | PVSLQCRLSMCPARSLLLVATLVLL |
| | | | | DHLSLARNLPVATPDPGMFPCLHHS |
| | | | | QNLLRAVSNMLQKARQTLEFYPCTS |
| | | | | EEIDHEDITKDKTSTVEACLPLELT |
| | | | | KNESCLNSRETSFITNGSCLASRKT |
| | | | | SEMMALCLSSIYEDLKMYQVEFKTM |
| | | | | NAKLLMDPKRQIFLDQNMLAVIDEL |
| | | | | MQALNFNSETVPQKSSLEEPDFYKT |
| | | | | KIKLCILLHAFRIRAVTIDRVMSYL |
| | | | | NAS |
| 102 | Linker | | | SGGGGSGGGGSGGGGSGGGGSG |
| 103 | Tri-functional | | | MDMRVPAQLLGLLQLWLSGARCDIV |
| | engager (IL-12) | | | MTQSPSSLSASVGDRVTITCKASQS |
| | including N- | | | VTNDVAWYQQKPGKAPKLLIYYASN |
| | terminal signal | | | RYTGVPDRFSGSGYGTDFTLTISSL |
| | sequence and C- | | | QPEDFATYFCQQDYRSPWTFGGGTK |
| | terminal linker- | | | VEIKGGGGSGGGGSGGGGSGGGSQV |
| | 6x-His tag | | | QLQESGPALVKPTQTLTLTCTVSGF |
| | | | | SLSDYGVNWIRQPPGKALEWLGIIW |
| | | | | GDGRTDYNSALKSRLSISKDNSKNQ |
| | | | | VVLRMTNVDPVDTATYFCARAPGNR |
| | | | | AMEYWGQGTVVTVSSASTSGGGGSG |
| | | | | GGGSGGGGSGGGGSGIWELKKDVYV |
| | | | | VELDWYPDAPGEMVVLTCDTPEEDG |
| | | | | ITWTLDQSSEVLGSGKTLTIQVKEF |
| | | | | GDAGQYTCHKGGEVLSHSLLLLHKK |
| | | | | EDGIWSTDILKDQKEPKNKTFLRCE |
| | | | | AKNYSGRFTCWWLTTISTDLTFSVK |
| | | | | SSRGSSDPQGVTCGAATLSAERVRG |
| | | | | DNKEYEYSVECQEDSACPAAEESLP |
| | | | | IEVMVDAVHKLKYENYTSSFFIRDI |
| | | | | IKPDPPKNLQLKPLKNSRQVEVSWE |
| | | | | YPDTWSTPHSYFSLTFCVQVQGKSK |
| | | | | REKKDRVFTDKTSATVICRKNASIS |
| | | | | VRAQDRYYSSSWSEWASVPCSGGGG |
| | | | | SGGGGSGGGGSRNLPVATPDPGMFP |
| | | | | CLHHSQNLLRAVSNMLQKARQTLEF |
| | | | | YPCTSEEIDHEDITKDKTSTVEACL |
| | | | | PLELTKNESCLNSRETSFITNGSCL |
| | | | | ASRKTSFMMALCLSSIYEDLKMYQV |
| | | | | EFKTMNAKLLMDPKRQIFLDQNMLA |
| | | | | VIDELMQALNFNSETVPQKSSLEEP |
| | | | | DFYKTKIKLCILLHAFRIRAVTIDR |
| | | | | VMSYLNASGSTSGSGKPGSGEGSTK |
| | | | | GQVQLQQSGAELARPGASVKMSCKT |
| | | | | SGYTFTRYTMHWVKQRPGQGLEWIG |
| | | | | YINPSRGYTNYNQKFKDKATLTTDK |
| | | | | SSSTAYMQLSSLTSEDSAVYYCARY |
| | | | | YDDHYSLDYWGQGTTLTVSSGGGGS |
| | | | | GGGGSGGGGSGGGGSQIVLTQSPAI |
| | | | | MSASPGEKVTMTCRASSSVSYMNWY |
| | | | | QQKSGTSPKRWIYDTSKVASGVPYR |
| | | | | FSGSGSGTSYSLTISSMEAEDAATY |
| | | | | YCQQWSSNPLTFGAGTKLELKGGSH |
| | | | | HHHHH |
| 104 | IL-12 Cytokine | | | IWELKKDVYVVELDWYPDAPGEMVV |
| | Domain | | | LTCDTPEEDGITWTLDQSSEVLGSG |
| | | | | KTLTIQVKEFGDAGQYTCHKGGEVL |
| | | | | SHSLLLLHKKEDGIWSTDILKDQKE |
| | | | | PKNKTFLRCEAKNYSGRFTCWWLTT |
| | | | | ISTDLTFSVKSSRGSSDPQGVTCGA |
| | | | | ATLSAERVRGDNKEYEYSVECQEDS |
| | | | | ACPAAEESLPIEVMVDAVHKLKYEN |
| | | | | YTSSFFIRDIIKPDPPKNLQLKPLK |
| | | | | NSRQVEVSWEYPDTWSTPHSYFSLT |
| | | | | FCVQVQGKSKREKKDRVFTDKTSAT |
| | | | | VICRKNASISVRAQDRYYSSSWSEW |
| | | | | ASVPCSGGGGSGGGGSGGGGSRNLP |
| | | | | VATPDPGMFPCLHHSQNLLRAVSNM |
| | | | | LQKARQTLEFYPCTSEEIDHEDITK |
| | | | | DKTSTVEACLPLELTKNESCLNSRE |
| | | | | TSFITNGSCLASRKTSFMMALCLSS |
| | | | | IYEDLKMYQVEFKTMNAKLLMDPKR |
| | | | | QIFLDQNMLAVIDELMQALNFNSET |
| | | | | VPQKSSLEEPDFYKTKIKLCILLHA |
| | | | | FRIRAVTIDRVMSYLNAS |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

One or more features from any embodiments described herein or in the figures may be combined with one or more features of any other embodiments described herein or in the figures without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A CD22xCD3 binding polypeptide comprising a CD22 binding domain and a CD3 binding domain,
a. wherein the CD22 binding domain comprises a first variable heavy chain comprising heavy chain complementarity determining region (HCDR) 1, HCDR2 and HCDR3 of SEQ ID NO: 1 and a first variable light chain comprising light chain complementarity determining region (LCDR) 1, LCDR2 and LCDR3 of SEQ ID NO: 8, and
b. wherein the CD3 binding domain comprises a second variable heavy chain comprising heavy chain complementarity determining region (HCDR) 1, HCDR2 and HCDR3 of SEQ ID NO: 16 and a second variable light chain comprising light chain complementarity determining region (LCDR) 1, LCDR2 and LCDR3 of SEQ ID NO: 23.

2. The CD22xCD3 binding polypeptide wherein the complementarity determining regions are according to the Kabat numbering scheme or according to the Chothia numbering scheme.

3. The CD22xCD3 binding polypeptide of claim 1, wherein the HCDR3 of the first variable heavy chain comprises the sequence of SEQ ID NOs: 2 or 5, wherein the HCDR2 of the first variable heavy chain comprises the sequence of SEQ ID NOs: 3 or 6, and wherein the HCDR1 of the first variable heavy chain comprises the sequence of SEQ ID NOs: 4 or 7.

4. The CD22xCD3 binding polypeptide of any one of claims 1-3, wherein the LCDR3 of the first variable light chain comprises the sequence of SEQ ID NOs: 9 or 12, wherein the LCDR2 of the first variable light chain comprises the sequence of SEQ ID NOs: 10 or 13, and wherein the LCDR1 of the first variable light chain comprises the sequence of SEQ ID NOs: 11 or 14.

5. The CD22xCD3 binding polypeptide of any one of the preceding claims, wherein the first variable heavy chain comprises a sequence having at least 80% sequence identity to the sequence of SEQ ID NO: 1.

6. The CD22xCD3 binding polypeptide of any one of claims 1-4, wherein the first variable heavy chain comprises the sequence of SEQ ID NO: 1.

7. The CD22xCD3 binding polypeptide of any one of the preceding claims, wherein the first variable light chain comprises a sequence having at least 80% sequence identity to the sequence of SEQ ID NO: 8.

8. The CD22xCD3 binding polypeptide of any one of claims 1-6, wherein the first variable light chain comprises the sequence of SEQ ID NO: 8.

9. The CD22xCD3 binding polypeptide of any one of the preceding claims, wherein the CD22 binding domain comprises a sequence having at least 80% sequence identity to SEQ ID NO: 15.

10. The CD22xCD3 binding polypeptide of any one of the preceding claims, wherein the HCDR3 of the second variable heavy chain comprises the sequence of SEQ ID NOs: 17 or 20, wherein the HCDR2 of the second variable heavy chain comprises the sequence of SEQ ID NOs: 18 or 21, and wherein the HCDR1 of the second variable heavy chain comprises the sequence of SEQ ID NOs: 19 or 22.

11. The CD22xCD3 binding polypeptide of any one of the preceding claims, wherein the LCDR3 of the second variable light chain comprises the sequence of SEQ ID NOs: 24 or 27, wherein the LCDR2 of the second variable light chain comprises the sequence of SEQ ID NOs: 25 or 28, and wherein the LCDR1 of the second variable light chain comprises the sequence of SEQ ID NOs: 26 or 29.

12. The CD22xCD3 binding polypeptide of any one of the preceding claims, wherein the second variable heavy chain comprises a sequence having at least 80% sequence identity to the sequence of SEQ ID NO: 16.

13. The CD22xCD3 binding polypeptide of any one of claims 1-11, wherein the second variable heavy chain comprises the sequence of SEQ ID NO: 16.

14. The CD22xCD3 binding polypeptide of any one of the preceding claims, wherein the second variable light chain comprises a sequence having at least 80% sequence identity to the sequence of SEQ ID NO: 23.

15. The CD22xCD3 binding polypeptide of any one of claims 1-13, wherein the second variable light chain comprises the sequence of SEQ ID NO: 23.

16. The CD22xCD3 binding polypeptide of any one of the preceding claims, wherein the CD3 binding domain comprises a sequence having at least 80% sequence identity to SEQ ID NO: 30.

17. The CD22xCD3 binding polypeptide of any one of the preceding claims, wherein the CD22 binding domain further comprises a first linker positioned between the first variable heavy chain and the first variable light chain.

18. The CD22xCD3 binding polypeptide of claim 17, wherein the first linker is selected from the group consisting of SEQ ID NO: 38, SEQ ID NO: 42, SEQ ID NO: 43, SEQ ID NO: 45, SEQ ID NO: 46, SEQ ID NO: 47, SEQ ID NO: 50, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99.

19. The CD22xCD3 binding polypeptide of claim 17, wherein the first linker comprises (G₄S)₄ (SEQ ID NO: 38).

20. The CD22xCD3 binding polypeptide of any one of the preceding claims, wherein the CD3 binding domain further comprises a second linker positioned between the second variable heavy chain and the second variable light chain.

21. The CD22xCD3 binding polypeptide of claim 20, wherein the second linker is selected from the group consisting of SEQ ID NO: 38, SEQ ID NO: 42, SEQ ID NO: 43, SEQ ID NO: 45, SEQ ID NO: 46, SEQ ID NO: 47, SEQ ID NO: 50, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99.

22. The CD22xCD3 binding polypeptide of claim 20, wherein the second linker comprises (G₄S)₄ (SEQ ID NO: 38).

23. The CD22xCD3 binding polypeptide of any one of the preceding claims, wherein the CD22 binding domain is linked to the CD3 binding domain by a third linker selected from the group consisting of SEQ ID NO: 38, SEQ ID NO: 39, SEQ ID NO: 40, SEQ ID NO: 41, SEQ ID NO: 42, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99.

24. The CD22xCD3 binding polypeptide of claim 23, wherein the third linker comprises SGGCGGS (SEQ ID NO: 41).

25. The CD22xCD3 binding polypeptide of any one of the preceding claims, wherein the CD3 binding domain is a scFv.

26. The CD22xCD3 binding polypeptide of any one of the preceding claims, wherein the CD22 binding domain is a scFv.

27. The CD22xCD3 binding polypeptide of any one of the preceding claims, wherein the first variable heavy chain and the first variable light chain are oriented in order from N- to C-terminus.

28. The CD22xCD3 binding polypeptide of any one of claims 1-26, wherein the first variable light chain and the first variable heavy chain are oriented in order from N- to C-terminus.

29. The CD22xCD3 binding polypeptide of any one of the preceding claims, wherein the second variable heavy chain and the second variable light chain are oriented in order from N- to C-terminus.

30. The CD22xCD3 binding polypeptide of any one of claims 1-28, wherein the second variable light chain and the second variable heavy chain are oriented in order from N- to C-terminus.

31. The CD22xCD3 binding polypeptide of any one of the preceding claims wherein the CD22 binding domain and the CD3 binding domain are oriented in order from N- to C-terminus.

32. The CD22xCD3 binding polypeptide of any one of claims 1-30, wherein the CD3 binding domain and the CD22 binding domain are oriented in order from N- to C-terminus.

33. The CD22xCD3 binding polypeptide of any one of the preceding claims, wherein the CD22 binding domain comprises the sequence of SEQ ID NO: 15.

34. The CD22xCD3 binding polypeptide of any one of the preceding claims, wherein the CD3 binding domain comprises the sequence of SEQ ID NO: 30.

35. The CD22xCD3 binding polypeptide of any one of claims 1-26, wherein the CD22xCD3 binding polypeptide comprises the sequence of SEQ ID NO: 31.

36. The CD22xCD3 binding polypeptide of any one of claims 1-26, wherein the CD22xCD3 binding polypeptide comprises the sequence of SEQ ID NO: 91.

37. A CD22xCD3 binding polypeptide comprising a CD22 binding domain and a CD3 binding domain, wherein
a. the CD22 binding domain comprises a first variable heavy chain comprising a sequence having at least 80% sequence identity to SEQ ID NO: 1 and a first variable light chain comprising a sequence having at least 80% sequence identity to SEQ ID NO: 8, and
b. wherein the CD3 binding domain comprises a second variable heavy chain comprising a sequence having at least 80% sequence identity to SEQ ID NO: 16 and a second variable light chain comprising a sequence having at least 80% sequence identity to SEQ ID NO: 23.

38. The CD22xCD3 binding polypeptide of claim 37, wherein the first variable heavy chain comprises the sequence of SEQ ID NO: 1.

39. The CD22xCD3 binding polypeptide of any one of claims 37-38, wherein the first variable light chain comprises the sequence of SEQ ID NO: 8.

40. The CD22xCD3 binding polypeptide of any one of claims 37-39, wherein the CD22 binding domain comprises a sequence having at least 80% sequence identity to SEQ ID NO: 15.

41. The CD22xCD3 binding polypeptide of any one of claims 37-40, wherein the second variable heavy chain comprises the sequence of SEQ ID NO: 16.

42. The CD22xCD3 binding polypeptide of any one of claims 37-41, wherein the second variable light chain comprises the sequence of SEQ ID NO: 23.

43. The CD22xCD3 binding polypeptide of any one of claims 37-42, wherein the CD3 binding domain comprises a sequence having at least 80% sequence identity to SEQ ID NO: 30.

44. The CD22xCD3 binding polypeptide of any one of claims 37-43, wherein the CD3 binding domain further comprises a first linker positioned between the first variable heavy chain and the first variable light chain.

45. The CD22xCD3 binding polypeptide of claim 44, wherein the first linker is selected from the group consisting of SEQ ID NO: 38, SEQ ID NO: 42, SEQ ID NO: 43, SEQ ID NO: 45, SEQ ID NO: 46, SEQ ID NO: 47, SEQ ID NO: 50, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99.

46. The CD22xCD3 binding polypeptide of claim 44, wherein the first linker comprises (G₄S)₄ (SEQ ID NO: 38).

47. The CD22xCD3 binding polypeptide of any one of claims 37-46, wherein the CD22 binding domain further comprises a second linker positioned between the second variable heavy chain and the second variable light chain.

48. The CD22xCD3 binding polypeptide of claim 47, wherein the second linker is selected from the group consisting of SEQ ID NO: 38, SEQ ID NO: 42, SEQ ID NO: 43, SEQ ID NO: 45, SEQ ID NO: 46, SEQ ID NO: 47, SEQ ID NO: 50, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99.

49. The CD22xCD3 binding polypeptide of claim 47, wherein the second linker comprises (G₄S)₄ (SEQ ID NO: 38).

50. The CD22xCD3 binding polypeptide of any one of claims 37-49, wherein the CD3 binding domain is linked to the CD22 binding domain by a third linker selected from the group consisting of SEQ ID NO: 38, SEQ ID NO: 39, SEQ ID NO: 40, SEQ ID NO: 41, SEQ ID NO: 42, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99.

51. The CD22xCD3 binding polypeptide of claim 50, wherein the third linker comprises SGGCGGS (SEQ ID NO: 41).

52. The CD22xCD3 binding polypeptide of any one of claims 37-51, wherein the CD3 binding domain is a scFv.

53. The CD22xCD3 binding polypeptide of any one of claims 37-52, wherein the CD22 binding domain is a scFv.

54. The CD22xCD3 binding polypeptide of any one of the preceding claims, wherein the first variable heavy chain and the first variable light chain are oriented in order from N- to C-terminus.

55. The CD22xCD3 binding polypeptide of any one of claims 37-53, wherein the first variable light chain and the first variable heavy chain are oriented in order from N- to C-terminus.

56. The CD22xCD3 binding polypeptide of any one of claims 37-55, wherein the second variable heavy chain and the second variable light chain are oriented in order from N- to C-terminus.

57. The CD22xCD3 binding polypeptide of any one of claims 37-56, wherein the second variable light chain and the second variable heavy chain are oriented in order from N- to C-terminus.

58. The CD22xCD3 binding polypeptide of any one of claims 37-57, wherein the CD22 binding domain and the CD3 binding domain are oriented in order from N- to C-terminus.

59. The CD22xCD3 binding polypeptide of any one of claims 37-57, wherein the CD3 binding domain and the CD22 binding domain are oriented in order from N- to C-terminus.

60. The CD22xCD3 binding polypeptide of any one of claims 37-59, wherein the CD22 binding domain comprises the sequence of SEQ ID NO: 15.

61. The CD22xCD3 binding polypeptide of any one of claims 37-60, wherein the CD3 binding domain comprises the sequence of SEQ ID NO: 30.

62. The CD22xCD3 binding polypeptide of any one of the preceding claims, wherein the CD22 binding domain is capable of blocking binding of the natural ligand to CD22.

63. A CD22xCD3 binding polypeptide comprising a CD22 binding domain and a CD3 binding domain, wherein the CD22 binding domain is capable of blocking binding of the natural ligand to CD22.

64. The CD22xCD3 binding polypeptide of any one of the preceding claims, further comprising a cytokine domain comprising a cytokine, a mutant, variant or functional fragment thereof.

65. The CD22xCD3 binding polypeptide of claim 64, further comprising a fourth linker between the CD22 binding domain and the cytokine domain.

66. The CD22xCD3 binding polypeptide of claim 65, wherein the fourth linker is selected from the group consisting of SEQ ID NO: 38, SEQ ID NOs: 42-51, SEQ ID NOs: 84-89, SEQ ID NOs: 92-94, and SEQ ID NO: 99.

67. The CD22xCD3 binding polypeptide of claim 65, wherein the fourth linker comprises SG₄SG₄SG₄SG₄SG (SEQ ID NO: 46).

68. The CD22xCD3 binding polypeptide of any one of claims 64-67, further comprising a fifth linker between the CD3 binding domain and the cytokine domain.

69. The CD22xCD3 binding polypeptide of claim 68, wherein the fifth linker is selected from the group consisting of SEQ ID NO: 38, SEQ ID NOs: 42-51, SEQ ID NOs: 84-89, SEQ ID NOs: 92-94, and SEQ ID NO: 99.

70. The CD22xCD3 binding polypeptide of claim 68, wherein the fifth linker comprises GSTSGSGKPGSGEGSTKG (SEQ ID NO: 47).

71. The CD22xCD3 binding polypeptide of any one of claims 64-70, wherein the cytokine is IL-2, IL-7, IL-12, IL-15, IL-18, IL-21, IL-23, interferon-α, interferon-β, interferon-γ and tumor necrosis factor α or a mutant, variant or functional fragment thereof.

72. The CD22xCD3 binding polypeptide of any one of claims 64-71, wherein the cytokine is IL-12 or a mutant, variant or functional fragment thereof.

73. The CD22xCD3 binding polypeptide of claim 72, wherein the IL-12, mutant, variant or functional fragment thereof comprises the sequences of SEQ ID NO: 97 and SEQ ID NO: 98, the sequences of SEQ ID NO: 100 and SEQ ID NO: 101, the sequences of amino acids 23-329 of SEQ ID NO: 100 and amino acids 57-253 of SEQ ID NO: 101, or the sequence of SEQ ID NO: 104.

74. The CD22xCD3 binding polypeptide of any one of claims 64-73, wherein the cytokine is IL-15 or a mutant, variant or functional fragment thereof.

75. The CD22xCD3 binding polypeptide of claim 74, wherein the IL-15, mutant, variant or functional fragment thereof comprises the sequence of SEQ ID NO: 34, SEQ ID NO: 35 or SEQ ID NO: 36.

76. The CD22xCD3 binding polypeptide of any one of claims 64-75, wherein the cytokine domain further comprises a sushi domain.

77. The CD22xCD3 binding polypeptide of claim 76, wherein the sushi domain comprises the sequence of SEQ ID NO: 32 or SEQ ID NO: 33.

78. The CD22xCD3 binding polypeptide of claim 76 or 77, wherein the IL-15, mutant, variant or fragment thereof is fused to the sushi domain by a sixth linker.

79. The CD22xCD3 binding polypeptide of claim 78, wherein the sixth linker is selected from the group consisting of SEQ ID NO: 38, SEQ ID NO: 42, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99.

80. The CD22xCD3 binding polypeptide of claim 78, wherein the sixth linker comprises G₃SG₄SG₃SG₄SLQ (SEQ ID NO: 51).

81. The CD22xCD3 binding polypeptide of any one of claims 63-71 and 74-80, wherein the cytokine domain comprises a sushi-IL-15 fusion protein comprises a sequence having at least 80% sequence identity to SEQ ID NO: 37.

82. The CD22xCD3 binding polypeptide of claim 81, wherein the cytokine domain comprises a sushi-IL-15 fusion protein comprising the sequence of SEQ ID NO: 37.

83. A CD22xCD3 binding polypeptide comprising the sequence of SEQ ID NO: 52.

84. A CD22xCD3 binding polypeptide comprising the sequence of SEQ ID NO: 96.

85. The CD22xCD3 binding polypeptide of any one of claims 1-63, wherein the CD22xCD3 binding polypeptide is a bispecific antibody.

86. A pharmaceutical composition comprising the CD22xCD3 binding polypeptide of any one of claims 1-85 and a pharmaceutically acceptable carrier.

87. A multispecific binding polypeptide comprising a first binding domain that specifically binds to CD3, a second binding domain that specifically binds to CD22, and a cytokine domain comprising a cytokine or a mutant, variant, or functional fragment thereof.

88. The multispecific binding polypeptide of claim 87, wherein the first binding domain is a scFv, Fab, F(ab′)2, or scFv-Fc.

89. The multispecific binding polypeptide of any one of claims 87-88, wherein the second binding domain is a scFv, Fab, F(ab′)2, or scFv-Fc.

90. The multispecific binding polypeptide of any one of claims 87-89, further comprising a fourth linker between the first binding domain and the cytokine domain.

91. The multispecific binding polypeptide of claim 90, wherein the fourth linker is selected from the group consisting of SEQ ID NO: 38, SEQ ID NOs: 42-51, SEQ ID NOs: 84-89, SEQ ID NOs: 92-94, and SEQ ID NO: 99.

92. The multispecific binding polypeptide of claim 90, wherein the fourth linker comprises SG₄SG₄SG₄SG₄SG (SEQ ID NO: 46).

93. The multispecific binding polypeptide of any one of claims 87-92, further comprising a fifth linker between the second binding domain and the cytokine domain.

94. The multispecific binding polypeptide of claim 93, wherein the fifth linker is selected from the group consisting of SEQ ID NO: 38, SEQ ID NOs: 42-51, SEQ ID NOs: 84-89, SEQ ID NOs: 92-94, and SEQ ID NO: 99.

95. The multispecific binding polypeptide of claim 93, wherein the fifth linker comprises GSTSGSGKPGSGEGSTKG (SEQ ID NO: 47).

96. The multispecific binding polypeptide of any one of claims 87-95, wherein the cytokine is IL IL-2, IL-7, IL-12, IL-15, IL-18, IL-21, IL-23, interferon-α, interferon-β, interferon-γ and tumor necrosis factor α or a mutant, variant, or functional fragment thereof.

97. The multispecific binding polypeptide of any one of claims 87-96, wherein the cytokine is IL-12 or a mutant, variant or functional fragment thereof.

98. The multispecific binding polypeptide of claim 97, wherein the IL-12, mutant, variant or functional fragment thereof comprises the sequences of SEQ ID NO: 97 and SEQ ID NO: 98, the sequences of SEQ ID NO: 100 and SEQ ID NO: 101, or the sequences of amino acids 23-329 of SEQ ID NO: 100 and amino acids 57-253 of SEQ ID NO: 101.

99. The multispecific binding polypeptide of any one of claims 87-96, wherein the cytokine is IL-15 or a mutant, variant or functional fragment thereof.

100. The multispecific binding polypeptide of claim 99, wherein the IL-15, mutant, variant or functional fragment thereof comprises the sequence of SEQ ID NO: 34, SEQ ID NO: 35 or SEQ ID NO: 36.

101. The multispecific binding polypeptide of any one of claims 87-96 and 99-100, wherein the cytokine domain further comprises a sushi domain.

102. The multispecific binding polypeptide of claim 101, wherein the sushi domain comprises the sequence of SEQ ID NO: 32 or SEQ ID NO: 33.

103. The multispecific binding polypeptide of claim 101 or 102, wherein the IL-15, mutant, variant or fragment thereof is fused to the sushi domain by a sixth linker.

104. The multispecific binding polypeptide of claim 103, wherein the sixth linker is selected from the group consisting of SEQ ID NO: 38, SEQ ID NO: 42, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 84, SEQ ID NO: 85, SEQ ID NO: 86, SEQ ID NO: 87, SEQ ID NO: 88, SEQ ID NO: 89, SEQ ID NO: 92, SEQ ID NO: 93, SEQ ID NO: 94, and SEQ ID NO: 99.

105. The multispecific binding polypeptide of claim 103, wherein the sixth linker comprises G₃SG₄SG₃SG₄SLQ (SEQ ID NO: 51).

106. The multispecific binding polypeptide of any one of claims 87-96 and 99-105, wherein the cytokine domain comprises a sushi-IL-15 fusion protein comprises a sequence having at least 80% sequence identity to SEQ ID NO: 37.

107. The multispecific binding polypeptide of claim 106, wherein the cytokine domain comprises a sushi-IL-15 fusion protein comprising the sequence of SEQ ID NO: 37.

108. The multispecific binding polypeptide of claim 87, wherein the first binding domain comprises a sequence having at least 80% sequence identity to SEQ ID NO: 30.

109. The multispecific binding polypeptide of claim 87, wherein the first binding domain comprises a sequence having at least 80% sequence identity to SEQ ID NO: 15.

110. A pharmaceutical composition comprising the multispecific binding polypeptide of any one of claims 87-109 and a pharmaceutically acceptable carrier.

111. A polynucleotide encoding the CD22xCD3 binding polypeptide of any one of claims 1-85 or the multispecific binding polypeptide of any one of claims 87-109.

112. An expression vector comprising the polynucleotide of claim 111.

113. A host cell comprising the polynucleotide of claim 111 or the expression vector of claim 112.

114. A method of treating cancer in a subject in need thereof, comprising:
administering a therapeutically effective amount of the CD22xCD3 binding polypeptide of any one of claims 1-85 or the multispecific binding polypeptide of any one of claims 86-109 to the subject.

115. The method of claim 114, wherein the cancer is selected from the group consisting of prostate cancer, lung cancer, colon cancer, rectum cancer, urinary bladder cancer, melanoma, kidney cancer, renal cancer, oral cavity cancer, pharynx cancer, pancreas cancer, uterine cancer, thyroid cancer, skin cancer, head and neck cancer, cervical cancer, breast cancer, ovarian cancer, sarcoma and hematopoietic cancer.

116. The method of claim 114, wherein the cancer is acute lymphocytic leukemia.

117. The method of claim 114, wherein the cancer is non-Hodgkin's lymphoma.

118. The method of any one of claims 114-117, further comprises administering a therapeutically effective amount of blinatumomab, rituximab, an antibody therapy, an antibody drug conjugate therapy, an additional therapeutic agent or CAR T cells to the subject.

119. A method for producing the CD22xCD3 binding polypeptide of any one of claims 1-85 or the multispecific binding polypeptide of any one of claims 87-109, comprising:
incubating the host cell of claim 113 comprising the polynucleotide of claim 111 or the expression vector of claim 112 under conditions sufficient to yield the CD22xCD3 binding polypeptide or multispecific binding polypeptide.


SEQUENCE LISTING (104 sequences)

SEQ ID NO: 1 - protein, 120 aa, synthetic construct
  REGION 1..120: note = Synthetic: HB22.7 VH
QVQLQESGPALVKPTQTLTLTCTVSGFSLSDYGVNWIRQPPGKALEWLGIIWGDGRTDYNSALKSRLSISKDNSKNQVVLRMTNVDPVDTATYFCARAPGNRAMEYWGQGTVVTVSSAST

SEQ ID NO: 2 - protein, 9 aa, synthetic construct
  REGION 1..9: note = Synthetic: HB22.7
APGNRAMEY

SEQ ID NO: 3 - protein, 16 aa, synthetic construct
  REGION 1..16: note = Synthetic: HB22.7
IIWGDGRTDYNSALKS

SEQ ID NO: 4 - protein, 5 aa, synthetic construct
  REGION 1..5: note = Synthetic: HB22.7
DYGVN

SEQ ID NO: 5 - protein, 9 aa, synthetic construct
  REGION 1..9: note = Synthetic: HB22.7
APGNRAMEY

SEQ ID NO: 6 - protein, 5 aa, synthetic construct
  REGION 1..5: note = Synthetic: HB22.7
WGDGR

SEQ ID NO: 7 - protein, 7 aa, synthetic construct
  REGION 1..7: note = Synthetic: HB22.7
GFSLSDY

SEQ ID NO: 8 - protein, 107 aa, synthetic construct
  REGION 1..107: note = Synthetic: HB22.7 VL
DIVMTQSPSSLSASVGDRVTITCKASQSVTNDVAWYQQKPGKAPKLLIYYASNRYTGVPDRFSGSGYGTDFTLTISSLQPEDFATYFCQQDYRSPWTFGGGTKVEIK

SEQ ID NO: 9 - protein, 9 aa, synthetic construct
  REGION 1..9: note = Synthetic: HB22.7
QQDYRSPWT

SEQ ID NO: 10 - protein, 7 aa, synthetic construct
  REGION 1..7: note = Synthetic: HB22.7
YASNRYT

SEQ ID NO: 11 - protein, 11 aa, synthetic construct
  REGION 1..11: note = Synthetic: HB22.7
KASQSVTNDVA

SEQ ID NO: 12 - protein, 9 aa, synthetic construct
  REGION 1..9: note = Synthetic: HB22.7
QQDYRSPWT

SEQ ID NO: 13 - protein, 7 aa, synthetic construct
  REGION 1..7: note = Synthetic: HB22.7
YASNRYT

SEQ ID NO: 14 - protein, 11 aa, synthetic construct
  REGION 1..11: note = Synthetic: HB22.7
KASQSVTNDVA

SEQ ID NO: 15 - protein, 247 aa, synthetic construct
  REGION 1..247: note = Synthetic: HB22.7 scFv (VL-linker-VH)
DIVMTQSPSSLSASVGDRVTITCKASQSVTNDVAWYQQKPGKAPKLLIYYASNRYTGVPDRFSGSGYGTDFTLTISSLQPEDFATYFCQQDYRSPWTFGGGTKVEIKGGGGSGGGGSGGGGSGGGGSQVQLQESGPALVKPTQTLTLTCTVSGFSLSDYGVNWIRQPPGKALEWLGIIWGDGRTDYNSALKSRLSISKDNSKNQVVLRMTNVDPVDTATYFCARAPGNRAMEYWGQGTVVTVSSAST

SEQ ID NO: 16 - protein, 119 aa, synthetic construct
  REGION 1..119: note = Synthetic: TR66 VH
QVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 17 - protein, 10 aa, synthetic construct
  REGION 1..10: note = Synthetic: TR66
YYDDHYSLDY

SEQ ID NO: 18 - protein, 17 aa, synthetic construct
  REGION 1..17: note = Synthetic: TR66
YINPSRGYTNYNQKFKD

SEQ ID NO: 19 - protein, 5 aa, synthetic construct
  REGION 1..5: note = Synthetic: TR66
RYTMH

SEQ ID NO: 20 - protein, 10 aa, synthetic construct
  REGION 1..10: note = Synthetic: TR66
YYDDHYSLDY

SEQ ID NO: 21 - protein, 6 aa, synthetic construct
  REGION 1..6: note = Synthetic: TR66
NPSRGY

SEQ ID NO: 22 - protein, 7 aa, synthetic construct
  REGION 1..7: note = Synthetic: TR66
GYTFTRY

SEQ ID NO: 23 - protein, 106 aa, synthetic construct
  REGION 1..106: note = Synthetic: TR66 VL
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 24 - protein, 9 aa, synthetic construct
  REGION 1..9: note = Synthetic: TR66
QQWSSNPLT

SEQ ID NO: 25 - protein, 7 aa, synthetic construct
  REGION 1..7: note = Synthetic: TR66
DTSKVAS

SEQ ID NO: 26 - protein, 10 aa, synthetic construct
  REGION 1..10: note = Synthetic: TR66
RASSSVSYMN

SEQ ID NO: 27 - protein, 9 aa, synthetic construct
  REGION 1..9: note = Synthetic: TR66
QQWSSNPLT

SEQ ID NO: 28 - protein, 7 aa, synthetic construct
  REGION 1..7: note = Synthetic: TR66
DTSKVAS

SEQ ID NO: 29 - protein, 10 aa, synthetic construct
  REGION 1..10: note = Synthetic: TR66
RASSSVSYMN

SEQ ID NO: 30 - protein, 245 aa, synthetic construct
  REGION 1..245: note = Synthetic: TR66 scFv (VH-linker-VL)
QVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 31 - protein, 499 aa, synthetic construct
  REGION 1..499: note = Synthetic: BiTE 197
DIVMTQSPSSLSASVGDRVTITCKASQSVTNDVAWYQQKPGKAPKLLIYYASNRYTGVPDRFSGSGYGTDFTLTISSLQPEDFATYFCQQDYRSPWTFGGGTKVEIKGGGGSGGGGSGGGGSGGGGSQVQLQESGPALVKPTQTLTLTCTVSGFSLSDYGVNWIRQPPGKALEWLGIIWGDGRTDYNSALKSRLSISKDNSKNQVVLRMTNVDPVDTATYFCARAPGNRAMEYWGQGTVVTVSSASTSGGCGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 32 - protein, 65 aa, synthetic construct
  REGION 1..65: note = Synthetic: sushi domain (aa 31-95 of IL-15R-alpha)
ITCPPPMSVEHADIWVKSYSLYSRERYICNSGFKRKAGTSSLTECVLNKATNVAHWTTPSLKCIR

SEQ ID NO: 33 - protein, 77 aa, synthetic construct
  REGION 1..77: note = Synthetic: sushi domain (aa 31-107 of IL-15R-alpha)
ITCPPPMSVEHADIWVKSYSLYSRERYICNSGFKRKAGTSSLTECVLNKATNVAHWTTPSLKCIRDPALVHQRPAPP

SEQ ID NO: 34 - protein, 162 aa, Homo sapiens
  SITE 1..162: note = human IL-15 (P40933)
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 35 - protein, 114 aa, Homo sapiens
  SITE 1..114: note = human IL-15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 36 - protein, 114 aa, synthetic construct
  REGION 1..114: note = Synthetic: IL-15 mutein (N72D)
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 37 - protein, 211 aa, synthetic construct
  REGION 1..211: note = Synthetic: sushi domain-IL-15 fusion including linker
ITCPPPMSVEHADIWVKSYSLYSRERYICNSGFKRKAGTSSLTECVLNKATNVAHWTTPSLKCIRDPALVHQRPAPPGGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 38 - protein, 20 aa, synthetic construct
  REGION 1..20: note = Synthetic: (G4S)4 linker
GGGGSGGGGSGGGGSGGGGS

SEQ ID NO: 39 - protein, 16 aa, synthetic construct
  REGION 1..16: note = Synthetic: Linker
GGGGSGGGGSCGGGGS

SEQ ID NO: 40 - protein, 20 aa, synthetic construct
  REGION 1..20: note = Synthetic: Linker
GGGGSGGGGCGGGGSGGGGS

SEQ ID NO: 41 - protein, 7 aa, synthetic construct
  REGION 1..7: note = Synthetic: Linker
SGGCGGS

SEQ ID NO: 42 - protein, 6 aa, synthetic construct
  REGION 1..6: note = Synthetic: Linker
SGGGGS

SEQ ID NO: 43 - protein, 15 aa, synthetic construct
  REGION 1..15: note = Synthetic: Linker
GGGGSGGGGSGGGGS

SEQ ID NO: 44 - protein, 21 aa, synthetic construct
  REGION 1..21: note = Synthetic: Linker
GGGGSGGGGSCGGGGSGGGGS

SEQ ID NO: 45 - protein, 6 aa, synthetic construct
  REGION 1..6: note = Synthetic: Linker
ASTGGS

SEQ ID NO: 46 - protein, 22 aa, synthetic construct
  REGION 1..22: note = Synthetic: Linker
SGGGGSGGGGSGGGGSGGGGSG

SEQ ID NO: 47 - protein, 18 aa, synthetic construct
  REGION 1..18: note = Synthetic: Linker
GSTSGSGKPGSGEGSTKG

SEQ ID NO: 48 - protein, 20 aa, synthetic construct
  REGION 1..20: note = Synthetic: Linker
PSGQAGAAASESLFVSNHAY

SEQ ID NO: 49 - protein, 7 aa, synthetic construct
  REGION 1..7: note = Synthetic: Linker
EASGGPE

SEQ ID NO: 50 - protein, 10 aa, synthetic construct
  REGION 1..10: note = Synthetic: Linker
GGGGSGGGGS

SEQ ID NO: 51 - protein, 20 aa, synthetic construct
  REGION 1..20: note = Synthetic: Linker
GGGSGGGGSGGGSGGGGSLQ

SEQ ID NO: 52 - protein, 743 aa, synthetic construct
  REGION 1..743: note = Synthetic: Tri-functional Engager 301
DIVMTQSPSSLSASVGDRVTITCKASQSVTNDVAWYQQKPGKAPKLLIYYASNRYTGVPDRFSGSGYGTDFTLTISSLQPEDFATYFCQQDYRSPWTFGGGTKVEIKGGGGSGGGGSGGGGSGGGGSQVQLQESGPALVKPTQTLTLTCTVSGFSLSDYGVNWIRQPPGKALEWLGIIWGDGRTDYNSALKSRLSISKDNSKNQVVLRMTNVDPVDTATYFCARAPGNRAMEYWGQGTVVTVSSASTSGGGGSGGGGSGGGGSGGGGSGITCPPPMSVEHADIWVKSYSLYSRERYICNSGFKRKAGTSSLTECVLNKATNVAHWTTPSLKCIRDPALVHQRPAPPGGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 53 - skipped

SEQ ID NO: 54 - protein, 2325 aa, synthetic construct
  REGION 1..2325: note = Synthetic: Tri-Functional Engager 301 (includes signal peptide and C-terminal GGS linker-His tag) (nucleotide sequence)
ATGGACATGAGGGTCCCTGCTCAGCTCCTGGGGCTCCTGCAGCTCTGGCTCTCAGGTGCCAGATGTGACATCGTGATGACCCAGTCTCCATCCTCCCTGTCTGCATCTGTAGGAGACAGAGTCACCATCACTTGCAAGGCCAGTCAGAGTGTGACTAATGATGTAGCTTGGTATCAGCAGAAACCAGGGAAAGCCCCTAAGCTCCTGATCTACTATGCATCCAATCGCTACACTGGGGTCCCAGATAGGTTCAGTGGAAGTGGATATGGGACAGATTTTACTTTGACCATCAGCAGCCTGCAGCCTGAAGATTTTGCAACATATTTCTGTCAGCAGGATTATAGGTCTCCGTGGACGTTTGGCGGCGGTACCAAAGTGGAGATCAAAGGCGGCGGCGGATCTGGTGGCGGAGGGTCAGGGGGAGGCGGTAGTGGTGGCGGAGGGAGCCAGGTCCAGTTGCAGGAGTCTGGTCCTGCGCTGGTGAAACCCACACAGACCCTCACACTGACCTGCACCGTGTCTGGGTTCTCACTCAGCGACTATGGTGTAAACTGGATCCGTCAGCCCCCAGGGAAGGCCCTGGAGTGGCTTGGAATAATATGGGGTGATGGAAGGACAGACTATAATTCAGCTCTCAAATCCAGGCTCAGCATCTCCAAGGACAACTCCAAAAACCAGGTGGTCCTTAGAATGACCAACGTGGACCCTGTGGACACAGCCACGTATTTCTGTGCAAGAGCCCCCGGTAATAGGGCTATGGAGTACTGGGGCCAGGGAACCGTGGTCACCGTCTCCTCAGCCTCCACCTCCGGCGGCGGAGGAAGCGGTGGGGGCGGTTCTGGAGGCGGTGGTAGTGGCGGAGGAGGCAGTGGAATCACGTGCCCTCCCCCCATGTCCGTGGAACACGCAGACATCTGGGTCAAGAGCTACAGCTTGTACTCCAGGGAGCGGTACATTTGTAACTCTGGTTTCAAGCGTAAAGCCGGCACGTCCAGCCTGACGGAGTGCGTGTTGAACAAGGCCACGAATGTCGCCCACTGGACAACCCCCAGTCTCAAATGCATTAGAGACCCTGCCCTGGTTCACCAAAGGCCAGCGCCACCCGGCGGAGGATCAGGCGGGGGCGGTAGTGGTGGCGGTTCTGGTGGTGGAGGTTCATTGCAAAATTGGGTAAATGTGATCTCTGACCTGAAGAAGATTGAGGATCTCATCCAATCTATGCATATCGACGCCACCCTCTATACCGAAAGTGATGTCCACCCCAGCTGCAAGGTAACAGCAATGAAATGTTTTCTGCTCGAACTTCAAGTGATATCCTTGGAATCAGGCGACGCATCAATCCATGATACAGTCGAAAATCTCATCATTCTGGCAAACAATTCCCTCTCCAGCAATGGCAACGTCACTGAGTCAGGATGCAAAGAGTGCGAAGAGCTCGAAGAAAAGAATATCAAGGAGTTCCTGCAAAGCTTTGTTCATATCGTCCAAATGTTCATTAATACATCCGGGAGTACGTCAGGATCTGGGAAGCCAGGCTCAGGAGAGGGCTCTACAAAGGGACAGGTGCAGCTGCAGCAGTCTGGCGCTGAGCTGGCTAGACCTGGCGCCTCCGTGAAGATGTCCTGCAAGACCTCCGGCTACACCTTCACCCGGTACACCATGCACTGGGTCAAGCAGAGGCCTGGACAGGGCCTGGAATGGATCGGCTACATCAACCCCTCCCGGGGCTACACCAACTACAACCAGAAGTTCAAGGACAAGGCCACCCTGACAACCGACAAGTCCTCCTCCACCGCCTACATGCAGCTGTCCTCCCTGACCTCCGAGGACTCCGCCGTGTACTACTGCGCCCGGTACTACGACGACCACTACTCCCTGGACTACTGGGGCCAGGGCACCACACTGACAGTGTCTAGCGGAGGCGGAGGATCTGGTGGTGGCGGATCTGGCGGCGGTGGAAGTGGCGGAGGTGGTAGCCAGATCGTGCTGACCCAGTCTCCCGCCATCATGTCTGCTAGCCCTGGCGAGAAAGTGACAATGACCTGCCGGGCCTCCTCCTCCGTGTCCTACATGAACTGGTATCAGCAGAAGTCCGGCACCTCCCCCAAGCGGTGGATCTACGACACCTCCAAGGTGGCCTCTGGCGTGCCCTACAGATTCTCCGGCTCTGGCTCTGGCACCTCCTACAGCCTGACCATCTCCAGCATGGAAGCCGAGGATGCCGCCACCTACTACTGCCAGCAGTGGTCCTCCAACCCCCTGACCTTTGGCGCTGGCACCAAGCTGGAACTGAAGGGCGGCTCTCATCACCATCATCACCATTGA

SEQ ID NO: 55 - protein, 847 aa, synthetic construct
  REGION 1..847: note = Synthetic: CD22
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 56 - protein, 207 aa, synthetic construct
  REGION 1..207: note = Synthetic: CD3E
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 57 - protein, 530 aa, synthetic construct
  REGION 1..530: note = Synthetic: BiTE 197 (including signal sequence and C-terminal GGS linker-His tag)
MDMRVPAQLLGLLQLWLSGARCDIVMTQSPSSLSASVGDRVTITCKASQSVTNDVAWYQQKPGKAPKLLIYYASNRYTGVPDRFSGSGYGTDFTLTISSLQPEDFATYFCQQDYRSPWTFGGGTKVEIKGGGGSGGGGSGGGGSGGGGSQVQLQESGPALVKPTQTLTLTCTVSGFSLSDYGVNWIRQPPGKALEWLGIIWGDGRTDYNSALKSRLSISKDNSKNQVVLRMTNVDPVDTATYFCARAPGNRAMEYWGQGTVVTVSSASTSGGCGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGSHHHHHH

SEQ ID NO: 58 - DNA, 1593 bp, synthetic construct
  misc_feature 1..1593: note = Synthetic: BiTE 197 (including signal sequence and C-terminal His tag) (nucleotide sequence)
  source 1..1593: mol_type = other DNA
atggacatgagggtccctgctcagctcctggggctcctgcagctctggctctcaggtgccagatgtgacatcgtgatgacccagtctccatcctccctgtctgcatctgtaggagacagagtcaccatcacttgcaaggccagtcagagtgtgactaatgatgtagcttggtatcagcagaaaccagggaaagcccctaagctcctgatctactatgcatccaatcgctacactggggtcccagataggttcagtggaagtggatatgggacagattttactttgaccatcagcagcctgcagcctgaagattttgcaacatatttctgtcagcaggattataggtctccgtggacgtttggcggcggtaccaaagtggagatcaaaggcggcggcggatctggtggcggagggtcagggggaggcggtagtggtggcggagggagccaggtccagttgcaggagtctggtcctgcgctggtgaaacccacacagaccctcacactgacctgcaccgtgtctgggttctcactcagcgactatggtgtaaactggatccgtcagcccccagggaaggccctggagtggcttggaataatatggggtgatggaaggacagactataattcagctctcaaatccaggctcagcatctccaaggacaactccaaaaaccaggtggtccttagaatgaccaacgtggaccctgtggacacagccacgtatttctgtgcaagagcccccggtaatagggctatggagtactggggccagggaaccgtggtcaccgtctcctcagcctccaccagtggcggctgtggaggatcccaggtgcagctgcagcagtctggcgctgagctggctagacctggcgcctccgtgaagatgtcctgcaagacctccggctacaccttcacccggtacaccatgcactgggtcaagcagaggcctggacagggcctggaatggatcggctacatcaacccctcccggggctacaccaactacaaccagaagttcaaggacaaggccaccctgacaaccgacaagtcctcctccaccgcctacatgcagctgtcctccctgacctccgaggactccgccgtgtactactgcgcccggtactacgacgaccactactccctggactactggggccagggcaccacactgacagtgtctagcggaggcggaggatctggtggtggcggatctggcggcggtggaagtggcggaggtggtagccagatcgtgctgacccagtctcccgccatcatgtctgctagccctggcgagaaagtgacaatgacctgccgggcctcctcctccgtgtcctacatgaactggtatcagcagaagtccggcacctcccccaagcggtggatctacgacacctccaaggtggcctctggcgtgccctacagattctccggctctggctctggcacctcctacagcctgaccatctccagcatggaagccgaggatgccgccacctactactgccagcagtggtcctccaaccccctgacctttggcgctggcaccaagctggaactgaagggcggctctcaccaccaccatcaccactga

SEQ ID NO: 59 - protein, 22 aa, synthetic construct
  REGION 1..22: note = Synthetic: BiTE 197/Tri-functional Engager 301/BiTE 198 signal sequence
MDMRVPAQLLGLLQLWLSGARC

SEQ ID NO: 60 - DNA, 66 bp, synthetic construct
  misc_feature 1..66: note = Synthetic: BiTE 197/Tri-functional Engager 301/BiTE 198 signal sequence (nucleotide sequence)
  source 1..66: mol_type = other DNA
atggacatgagggtccctgctcagctcctggggctcctgcagctctggctctcaggtgccagatgt

SEQ ID NO: 61 - DNA, 321 bp, synthetic construct
  misc_feature 1..321: note = Synthetic: HB22.7 VL (nucleotide sequence)
  source 1..321: mol_type = other DNA
gacatcgtgatgacccagtctccatcctccctgtctgcatctgtaggagacagagtcaccatcacttgcaaggccagtcagagtgtgactaatgatgtagcttggtatcagcagaaaccagggaaagcccctaagctcctgatctactatgcatccaatcgctacactggggtcccagataggttcagtggaagtggatatgggacagattttactttgaccatcagcagcctgcagcctgaagattttgcaacatatttctgtcagcaggattataggtctccgtggacgtttggcggcggtaccaaagtggagatcaaa

SEQ ID NO: 62 - DNA, 60 bp, synthetic construct
  misc_feature 1..60: note = Synthetic: (G4S)4 linker (nucleotide sequence)
  source 1..60: mol_type = other DNA
ggcggcggcggatctggtggcggagggtcagggggaggcggtagtggtggcggagggagc

SEQ ID NO: 63 - DNA, 360 bp, synthetic construct
  misc_feature 1..360: note = Synthetic: HB22.7 VH (nucleotide sequence)
  source 1..360: mol_type = other DNA
caggtccagttgcaggagtctggtcctgcgctggtgaaacccacacagaccctcacactgacctgcaccgtgtctgggttctcactcagcgactatggtgtaaactggatccgtcagcccccagggaaggccctggagtggcttggaataatatggggtgatggaaggacagactataattcagctctcaaatccaggctcagcatctccaaggacaactccaaaaaccaggtggtccttagaatgaccaacgtggaccctgtggacacagccacgtatttctgtgcaagagcccccggtaatagggctatggagtactggggccagggaaccgtggtcaccgtctcctcagcctccacc

SEQ ID NO: 64 - DNA, 21 bp, synthetic construct
  misc_feature 1..21: note = Synthetic: Linker SGGCGGS(nucleotide sequence)
  source 1..21: mol_type = other DNA
agtggcggctgtggaggatcc

SEQ ID NO: 65 - DNA, 357 bp, synthetic construct
  misc_feature 1..357: note = Synthetic: TR66 VH (nucleotide sequence)
  source 1..357: mol_type = other DNA
caggtgcagctgcagcagtctggcgctgagctggctagacctggcgcctccgtgaagatgtcctgcaagacctccggctacaccttcacccggtacaccatgcactgggtcaagcagaggcctggacagggcctggaatggatcggctacatcaacccctcccggggctacaccaactacaaccagaagttcaaggacaaggccaccctgacaaccgacaagtcctcctccaccgcctacatgcagctgtcctccctgacctccgaggactccgccgtgtactactgcgcccggtactacgacgaccactactccctggactactggggccagggcaccacactgacagtgtctagc

SEQ ID NO: 66 - DNA, 318 bp, synthetic construct
  misc_feature 1..318: note = Synthetic: TR66 VL (nucleotide sequence)
  source 1..318: mol_type = other DNA
cagatcgtgctgacccagtctcccgccatcatgtctgctagccctggcgagaaagtgacaatgacctgccgggcctcctcctccgtgtcctacatgaactggtatcagcagaagtccggcacctcccccaagcggtggatctacgacacctccaaggtggcctctggcgtgccctacagattctccggctctggctctggcacctcctacagcctgaccatctccagcatggaagccgaggatgccgccacctactactgccagcagtggtcctccaaccccctgacctttggcgctggcaccaagctggaactgaag

SEQ ID NO: 67 - protein, 774 aa, synthetic construct
  REGION 1..774: note = Synthetic: Tri-functional Engager 301 (including signal sequence and C-terminal GGS linker-His tag)
MDMRVPAQLLGLLQLWLSGARCDIVMTQSPSSLSASVGDRVTITCKASQSVTNDVAWYQQKPGKAPKLLIYYASNRYTGVPDRFSGSGYGTDFTLTISSLQPEDFATYFCQQDYRSPWTFGGGTKVEIKGGGGSGGGGSGGGGSGGGGSQVQLQESGPALVKPTQTLTLTCTVSGFSLSDYGVNWIRQPPGKALEWLGIIWGDGRTDYNSALKSRLSISKDNSKNQVVLRMTNVDPVDTATYFCARAPGNRAMEYWGQGTVVTVSSASTSGGGGSGGGGSGGGGSGGGGSGITCPPPMSVEHADIWVKSYSLYSRERYICNSGFKRKAGTSSLTECVLNKATNVAHWTTPSLKCIRDPALVHQRPAPPGGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGSHHHHHH

SEQ ID NO: 68 - DNA, 2325 bp, synthetic construct
  misc_feature 1..2325: note = Synthetic: Tri-functional Engager 301 (including signal sequence and His tag) (nucleotide sequence)
  source 1..2325: mol_type = other DNA
atggacatgagggtccctgctcagctcctggggctcctgcagctctggctctcaggtgccagatgtgacatcgtgatgacccagtctccatcctccctgtctgcatctgtaggagacagagtcaccatcacttgcaaggccagtcagagtgtgactaatgatgtagcttggtatcagcagaaaccagggaaagcccctaagctcctgatctactatgcatccaatcgctacactggggtcccagataggttcagtggaagtggatatgggacagattttactttgaccatcagcagcctgcagcctgaagattttgcaacatatttctgtcagcaggattataggtctccgtggacgtttggcggcggtaccaaagtggagatcaaaggcggcggcggatctggtggcggagggtcagggggaggcggtagtggtggcggagggagccaggtccagttgcaggagtctggtcctgcgctggtgaaacccacacagaccctcacactgacctgcaccgtgtctgggttctcactcagcgactatggtgtaaactggatccgtcagcccccagggaaggccctggagtggcttggaataatatggggtgatggaaggacagactataattcagctctcaaatccaggctcagcatctccaaggacaactccaaaaaccaggtggtccttagaatgaccaacgtggaccctgtggacacagccacgtatttctgtgcaagagcccccggtaatagggctatggagtactggggccagggaaccgtggtcaccgtctcctcagcctccacctccggcggcggaggaagcggtgggggcggttctggaggcggtggtagtggcggaggaggcagtggaatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccggcggaggatcaggcgggggcggtagtggtggcggttctggtggtggaggttcattgcaaaattgggtaaatgtgatctctgacctgaagaagattgaggatctcatccaatctatgcatatcgacgccaccctctataccgaaagtgatgtccaccccagctgcaaggtaacagcaatgaaatgttttctgctcgaacttcaagtgatatccttggaatcaggcgacgcatcaatccatgatacagtcgaaaatctcatcattctggcaaacaattccctctccagcaatggcaacgtcactgagtcaggatgcaaagagtgcgaagagctcgaagaaaagaatatcaaggagttcctgcaaagctttgttcatatcgtccaaatgttcattaatacatccgggagtacgtcaggatctgggaagccaggctcaggagagggctctacaaagggacaggtgcagctgcagcagtctggcgctgagctggctagacctggcgcctccgtgaagatgtcctgcaagacctccggctacaccttcacccggtacaccatgcactgggtcaagcagaggcctggacagggcctggaatggatcggctacatcaacccctcccggggctacaccaactacaaccagaagttcaaggacaaggccaccctgacaaccgacaagtcctcctccaccgcctacatgcagctgtcctccctgacctccgaggactccgccgtgtactactgcgcccggtactacgacgaccactactccctggactactggggccagggcaccacactgacagtgtctagcggaggcggaggatctggtggtggcggatctggcggcggtggaagtggcggaggtggtagccagatcgtgctgacccagtctcccgccatcatgtctgctagccctggcgagaaagtgacaatgacctgccgggcctcctcctccgtgtcctacatgaactggtatcagcagaagtccggcacctcccccaagcggtggatctacgacacctccaaggtggcctctggcgtgccctacagattctccggctctggctctggcacctcctacagcctgaccatctccagcatggaagccgaggatgccgccacctactactgccagcagtggtcctccaaccccctgacctttggcgctggcaccaagctggaactgaagggcggctctcatcaccatcatcaccattga

SEQ ID NO: 69 - DNA, 66 bp, synthetic construct
  misc_feature 1..66: note = Synthetic: Linker (SG4)4SG (nucleotide sequence)
  source 1..66: mol_type = other DNA
tccggcggcggaggaagcggtgggggcggttctggaggcggtggtagtggcggaggaggcagtgga

SEQ ID NO: 70 - DNA, 231 bp, synthetic construct
  misc_feature 1..231: note = Synthetic: Sushi domain (aa 31-107) (nucleotide sequence)
  source 1..231: mol_type = other DNA
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccc

SEQ ID NO: 71 - DNA, 60 bp, synthetic construct
  misc_feature 1..60: note = Synthetic: Linker (GGGSGGGGSGGGSGGGGSLQ) (nucleotide sequence)
  source 1..60: mol_type = other DNA
ggcggaggatcaggcgggggcggtagtggtggcggttctggtggtggaggttcattgcaa

SEQ ID NO: 72 - DNA, 342 bp, Homo sapiens
  misc_feature 1..342: note = Human IL-15 (nucleotide sequence)
  source 1..342: mol_type = genomic DNA
aattgggtaaatgtgatctctgacctgaagaagattgaggatctcatccaatctatgcatatcgacgccaccctctataccgaaagtgatgtccaccccagctgcaaggtaacagcaatgaaatgttttctgctcgaacttcaagtgatatccttggaatcaggcgacgcatcaatccatgatacagtcgaaaatctcatcattctggcaaacaattccctctccagcaatggcaacgtcactgagtcaggatgcaaagagtgcgaagagctcgaagaaaagaatatcaaggagttcctgcaaagctttgttcatatcgtccaaatgttcattaatacatcc

SEQ ID NO: 73 - DNA, 54 bp, synthetic construct
  misc_feature 1..54: note = Synthetic: Whitlow Linker (nucleotide sequence)
  source 1..54: mol_type = other DNA
gggagtacgtcaggatctgggaagccaggctcaggagagggctctacaaaggga

SEQ ID NO: 74 - protein, 114 aa, synthetic construct
  REGION 1..114: note = Synthetic: IL-15 mutein (N72A)
NWVNVISDLKKIEDLIQSMHIDATLYTESDVHPSCKVTAMKCFLLELQVISLESGDASIHDTVENLIILANASLSSNGNVTESGCKECEELEEKNIKEFLQSFVHIVQMFINTS

SEQ ID NO: 75 - protein, 114 aa, synthetic construct
  REGION 1..114: note = Synthetic: IL-15 mutein (N72E)
NWVNVISDLKKIEDLIQSMHIDATLYTESDVHPSCKVTAMKCFLLELQVISLESGDASIHDTVENLIILANESLSSNGNVTESGCKECEELEEKNIKEFLQSFVHIVQMFINTS

SEQ ID NO: 76 - protein, 114 aa, synthetic construct
  REGION 1..114: note = Synthetic: IL-15 mutein (N72S)
NWVNVISDLKKIEDLIQSMHIDATLYTESDVHPSCKVTAMKCFLLELQVISLESGDASIHDTVENLIILANSSLSSNGNVTESGCKECEELEEKNIKEFLQSFVHIVQMFINTS

SEQ ID NO: 77 - protein, 114 aa, synthetic construct
  REGION 1..114: note = Synthetic: IL-15 mutein (N72Y)
NWVNVISDLKKIEDLIQSMHIDATLYTESDVHPSCKVTAMKCFLLELQVISLESGDASIHDTVENLIILANYSLSSNGNVTESGCKECEELEEKNIKEFLQSFVHIVQMFINTS

SEQ ID NO: 78 - protein, 114 aa, synthetic construct
  REGION 1..114: note = Synthetic: IL-15 mutein (N72P)
NWVNVISDLKKIEDLIQSMHIDATLYTESDVHPSCKVTAMKCFLLELQVISLESGDASIHDTVENLIILANPSLSSNGNVTESGCKECEELEEKNIKEFLQSFVHIVQMFINTS

SEQ ID NO: 79 - protein, 599 aa, synthetic construct
  REGION 1..599: note = Synthetic: BiTE 198 (includes signal sequence and C-terminal GGS-His tag)
MDMRVPAQLLGLLQLWLSGARCDIVMTQSPSSLSASVGDRVTITCKASQSVTNDVAWYQQKPGKAPKLLIYYASNRYTGVPDRFSGSGYGTDFTLTISSLQPEDFATYFCQQDYRSPWTFGGGTKVEIKGGGGSGGGGSGGGGSGGGGSQVQLQESGPALVKPTQTLTLTCTVSGFSLSDYGVNWIRQPPGKALEWLGIIWGDGRTDYNSALKSRLSISKDNSKNQVVLRMTNVDPVDTATYFCARAPGNRAMEYWGQGTVVTVSSASTGGGGSGGGGSGGGGSLAEAKVLANRELDKYGVSDFYKRLINKAKTVEGVEALKLHILAALPGGGGSGGGGSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGSHHHHHH

SEQ ID NO: 80 - DNA, 1797 bp, synthetic construct
  misc_feature 1..1797: note = Synthetic: BiTE 198 (includes signal sequence and His tag) (nucleotide sequence)
  source 1..1797: mol_type = other DNA
atggacatgagggtccctgctcagctcctggggctcctgcagctctggctctcaggtgccagatgtgacatcgtgatgacccagtctccatcctccctgtctgcatctgtaggagacagagtcaccatcacttgcaaggccagtcagagtgtgactaatgatgtagcttggtatcagcagaaaccagggaaagcccctaagctcctgatctactatgcatccaatcgctacactggggtcccagataggttcagtggaagtggatatgggacagattttactttgaccatcagcagcctgcagcctgaagattttgcaacatatttctgtcagcaggattataggtctccgtggacgtttggcggcggtaccaaagtggagatcaaaggcggcggcggatctggtggcggagggtcagggggaggcggtagtggtggcggagggagccaggtccagttgcaggagtctggtcctgcgctggtgaaacccacacagaccctcacactgacctgcaccgtgtctgggttctcactcagcgactatggtgtaaactggatccgtcagcccccagggaaggccctggagtggcttggaataatatggggtgatggaaggacagactataattcagctctcaaatccaggctcagcatctccaaggacaactccaaaaaccaggtggtccttagaatgaccaacgtggaccctgtggacacagccacgtatttctgtgcaagagcccccggtaatagggctatggagtactggggccagggaaccgtggtcaccgtctcctcagcctccaccggaggaggcggctctggaggtggcggcagtggtggaggcgggtctctggccgaggccaaggtgctggccaacagagagctggacaagtacggcgtgagcgacttctacaagagactgatcaacaaggccaagaccgtggagggcgtggaggccctgaagctgcacatcctggccgccctgcccggcggtggcggatctggaggtggtgggagcggcggaggcggatctcaggtgcagctgcagcagtctggcgctgagctggctagacctggcgcctccgtgaagatgtcctgcaagacctccggctacaccttcacccggtacaccatgcactgggtcaagcagaggcctggacagggcctggaatggatcggctacatcaacccctcccggggctacaccaactacaaccagaagttcaaggacaaggccaccctgacaaccgacaagtcctcctccaccgcctacatgcagctgtcctccctgacctccgaggactccgccgtgtactactgcgcccggtactacgacgaccactactccctggactactggggccagggcaccacactgacagtgtctagcggaggcggaggatctggtggtggcggatctggcggcggtggaagtggcggaggtggtagccagatcgtgctgacccagtctcccgccatcatgtctgctagccctggcgagaaagtgacaatgacctgccgggcctcctcctccgtgtcctacatgaactggtatcagcagaagtccggcacctcccccaagcggtggatctacgacacctccaaggtggcctctggcgtgccctacagattctccggctctggctctggcacctcctacagcctgaccatctccagcatggaagccgaggatgccgccacctactactgccagcagtggtcctccaaccccctgacctttggcgctggcaccaagctggaactgaagggcggctctcaccaccaccatcaccac

SEQ ID NO: 81 - protein, 45 aa, synthetic construct
  REGION 1..45: note = Synthetic: (G4S)3 Linker
GGCGGTGGCGGATCTGGAGGTGGTGGGAGCGGCGGAGGCGGATCT

SEQ ID NO: 82 - protein, 46 aa, synthetic construct
  REGION 1..46: note = Synthetic: Albumin binding domain
LAEAKVLANRELDKYGVSDFYKRLINKAKTVEGVEALKLHILAALP

SEQ ID NO: 83 - DNA, 138 bp, synthetic construct
  misc_feature 1..138: note = Synthetic: Albumin binding domain (nucleotide sequence)
  source 1..138: mol_type = other DNA
ctggccgaggccaaggtgctggccaacagagagctggacaagtacggcgtgagcgacttctacaagagactgatcaacaaggccaagaccgtggagggcgtggaggccctgaagctgcacatcctggccgccctgccc

SEQ ID NO: 84 - protein, 8 aa, synthetic construct
  REGION 1..8: note = Synthetic: Linker (Gly8)
GGGGGGGG

SEQ ID NO: 85 - protein, 6 aa, synthetic construct
  REGION 1..6: note = Synthetic: Linker (Gly6)
GGGGGG

SEQ ID NO: 86 - protein, 5 aa, synthetic construct
  REGION 1..5: note = Synthetic: Linker
GGGGS

SEQ ID NO: 87 - protein, 18 aa, synthetic construct
  REGION 1..18: note = Synthetic: Linker
KESGSVSSEQLAQFRSLD

SEQ ID NO: 88 - protein, 14 aa, synthetic construct
  REGION 1..14: note = Synthetic: Linker
EGKSSGSGSESKST

SEQ ID NO: 89 - protein, 12 aa, synthetic construct
  REGION 1..12: note = Synthetic: Linker
GSAGSAAGSGEF

SEQ ID NO: 90 - DNA, 60 bp, synthetic construct
  misc_feature 1..60: note = Synthetic: Linker (G4S)4 (nucleotide sequence)
  source 1..60: mol_type = other DNA
ggaggcggaggatctggtggtggcggatctggcggcggtggaagtggcggaggtggtagc

SEQ ID NO: 91 - protein, 568 aa, synthetic construct
  REGION 1..568: note = Synthetic: BiTE 198 (without signal peptide or GGS-His tag)
DIVMTQSPSSLSASVGDRVTITCKASQSVTNDVAWYQQKPGKAPKLLIYYASNRYTGVPDRFSGSGYGTDFTLTISSLQPEDFATYFCQQDYRSPWTFGGGTKVEIKGGGGSGGGGSGGGGSGGGGSQVQLQESGPALVKPTQTLTLTCTVSGFSLSDYGVNWIRQPPGKALEWLGIIWGDGRTDYNSALKSRLSISKDNSKNQVVLRMTNVDPVDTATYFCARAPGNRAMEYWGQGTVVTVSSASTGGGGSGGGGSGGGGSLAEAKVLANRELDKYGVSDFYKRLINKAKTVEGVEALKLHILAALPGGGGSGGGGSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 92 - protein, 14 aa, synthetic construct
  REGION 1..14: note = Synthetic: Linker
GGGGSGGGGSGGSA

SEQ ID NO: 93 - protein, 10 aa, synthetic construct
  REGION 1..10: note = Synthetic: Linker
SGGGGSGGGG

SEQ ID NO: 94 - protein, 10 aa, synthetic construct
  REGION 1..10: note = Synthetic: Linker
SGSGGGGSLQ

SEQ ID NO: 95 - DNA, 3329 bp, synthetic construct
  misc_feature 1..3329: note = Synthetic: Tri-functional engager (IL-12) (nucleotide sequence)
  source 1..3329: mol_type = other DNA
gaccgatccagcctccggactctagaggatcgaacccttgccaccatggacatgagggtccctgctcagctcctggggctcctgcagctctggctctcaggtgccagatgtgacatcgtgatgacccagtctccatcctccctgtctgcatctgtaggagacagagtcaccatcacttgcaaggccagtcagagtgtgactaatgatgtagcttggtatcagcagaaaccagggaaagcccctaagctcctgatctactatgcatccaatcgctacactggggtcccagataggttcagtggaagtggatatgggacagattttactttgaccatcagcagcctgcagcctgaagattttgcaacatatttctgtcagcaggattataggtctccgtggacgtttggcggcggtaccaaagtggagatcaaaggcggcggcggatctggtggcggagggtcagggggaggcggtagtggcggagggagccaggtccagttgcaggagtctggtcctgcgctggtgaaacccacacagaccctcacactgacctgcaccgtgtctgggttctcactcagcgactatggtgtaaactggatccgtcagcccccagggaaggccctggagtggcttggaataatatggggtgatggaaggacagactataattcagctctcaaatccaggctcagcatctccaaggacaactccaaaaaccaggtggtccttagaatgaccaacgtggaccctgtggacacagccacgtatttctgtgcaagagcccccggtaatagggctatggagtactggggccagggaaccgtggtcaccgtctcctcagcctccacctccggcggcggaggaagcggtgggggcggttctggaggcggtggtagtggcggaggaggcagtggaatctgggagcttaaaaaggatgtatacgtagtcgagcttgattggtaccccgacgcgcctggggaaatggtggtgctcacatgtgataccccggaagaagacgggataacttggactcttgaccaaagctccgaggtgttggggagtggcaagacgctcacaatccaagtcaaggaattcggggacgccggtcaatacacatgtcacaagggtggcgaggtgctgagccactctcttcttcttttgcataaaaaggaggacggaatatggtccacagacatcctgaaggaccagaaagaaccgaaaaataaaacattcctcaggtgcgaggccaagaactattctggcagatttacgtgttggtggcttaccactatttcaaccgaccttactttcagtgtgaagagtagcagaggaagttcagaccctcaaggtgtcacctgcggtgccgccacgctgtcagcggaaagagttagaggggacaacaaggagtatgaatacagcgttgagtgtcaagaggatagcgcgtgtcccgccgctgaggagtcccttccgatagaggtgatggtcgatgcagtgcataaactcaaatacgagaactacacgagttctttcttcattagagatatcatcaaacctgatcctccgaaaaacctccaactcaagccgctcaaaaacagtcgccaggttgaagtcagttgggagtaccctgatacttggagcaccccacactcctatttctccctgacgttttgtgtacaggttcagggcaaaagtaaaagggaaaaaaaagaccgcgtgttcacagataagacctcagcaactgtcatttgtagaaaaaacgcatctattagcgtccgggcacaggaccggtattactcatcttcctggtcagagtgggcgagtgtgccttgctctggtggagggggatctggaggaggcggttcaggaggaggggggtccaggaatctgcctgtcgcgacgcccgacccgggtatgtttccgtgcttgcaccactcacaaaaccttctgcgcgccgtcagtaatatgttgcaaaaggctaggcaaacccttgaattttatccgtgcacctccgaagaaatagaccatgaggacattaccaaggacaagacgtctacagtggaggcctgcttgccccttgagctcacaaagaatgagagctgcttgaattcacgcgaaacatcttttatcacgaacgggagttgcctggcctcccggaagacgtcttttatgatggctctgtgtttgtcatcaatttacgaggatttgaagatgtaccaagtggaattcaagacaatgaatgcgaaactgctgatggacccgaagcgccaaatcttcctcgatcaaaatatgctcgctgtcatcgacgaactgatgcaggcgctcaacttcaattccgagacggtgccgcagaaaagtagcctggaagagccggatttctataaaacaaaaatcaaactgtgcatcttgctgcacgcatttaggattcgagctgtaactatcgatcgcgtaatgtcttatctgaacgctagtgggagtacgtcaggatctgggaagccaggctcaggagagggctctacaaagggacaggtgcagctgcagcagtctggcgctgagctggctagacctggcgcctccgtgaagatgtcctgcaagacctccggctacaccttcacccggtacaccatgcactgggtcaagcagaggcctggacagggcctggaatggatcggctacatcaacccctcccggggctacaccaactacaaccagaagttcaaggacaaggccaccctgacaaccgacaagtcctcctccaccgcctacatgcagctgtcctccctgacctccgaggactccgccgtgtactactgcgcccggtactacgacgaccactactccctggactactggggccagggcaccacactgacagtgtctagcggaggcggaggatctggtggtggcggatctggcggcggtggaagtggcggaggtggtagccagatcgtgctgacccagtctcccgccatcatgtctgctagccctggcgagaaagtgacaatgacctgccgggcctcctcctccgtgtcctacatgaactggtatcagcagaagtccggcacctcccccaagcggtggatctacgacacctccaaggtggcctctggcgtgccctacagattctccggctctggctctggcacctcctacagcctgaccatctccagcatggaagccgaggatgccgccacctactactgccagcagtggtcctccaaccccctgacctttggcgctggcaccaagctggaactgaagggcggctctcatcaccatcatcaccattgaaagggttcgatccctaccggttagtaatgagtttaaacggg

SEQ ID NO: 96 - protein, 1049 aa, synthetic construct
  REGION 1..1049: note = Synthetic: Tri-functional engager (IL-12)
DIVMTQSPSSLSASVGDRVTITCKASQSVTNDVAWYQQKPGKAPKLLIYYASNRYTGVPDRFSGSGYGTDFTLTISSLQPEDFATYFCQQDYRSPWTFGGGTKVEIKGGGGSGGGGSGGGGSGGGSQVQLQESGPALVKPTQTLTLTCTVSGFSLSDYGVNWIRQPPGKALEWLGIIWGDGRTDYNSALKSRLSISKDNSKNQVVLRMTNVDPVDTATYFCARAPGNRAMEYWGQGTVVTVSSASTSGGGGSGGGGSGGGGSGGGGSGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSTSGSGKPGSGEGSTKGQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 97 - protein, 306 aa, synthetic construct
  REGION 1..306: note = Synthetic: IL-12 p40
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 98 - protein, 197 aa, synthetic construct
  REGION 1..197: note = Synthetic: IL-12 p35
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 99 - protein, 14 aa, synthetic construct
  REGION 1..14: note = Synthetic: Linker
GGGGSGGGGSGGGS

SEQ ID NO: 100 - protein, 329 aa, Homo sapiens
  SITE 1..329: note = Human IL-12 p40
MLQQQLVLSCFSLVLLASPLAAIWELEKDVYVVELDWYPDAPGEMVVLTCDTPEEEGITWTSDQSSEVLGSGKTLTIQVKEFGDAGQYTCHKGDTILSQSLLLLHKKEDGIWSTDILKDQKEPKNKTFLKCKAKNYSGHFTCWWLTAISTDLKFSVKSSRGSSDPRGVTCGPATLSAERVRVDNREYKKYTVECQEGSSCPAAEESLPIEVLVDAVHKLKYENYTSSFFIRDIIKPDPPKNLQLKPLKNSRHVEVSWEYPDTWSTPHSYFSLTFSLQVQGKNKREKKDRLFMDQTSAKVTCHKDAVIRVQARDRYYSSSWSEWASVSCS

SEQ ID NO: 101 - protein, 253 aa, Homo sapiens
  SITE 1..253: note = Human IL-12 p35
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 102 - protein, 22 aa, synthetic construct
  REGION 1..22: note = Synthetic: Linker
SGGGGSGGGGSGGGGSGGGGSG

SEQ ID NO: 103 - protein, 1080 aa, synthetic construct
  REGION 1..1080: note = Synthetic: Tri-functional engager (IL-12) including N-terminal signal sequence and C-terminal linker-6x-His tag
MDMRVPAQLLGLLQLWLSGARCDIVMTQSPSSLSASVGDRVTITCKASQSVTNDVAWYQQKPGKAPKLLIYYASNRYTGVPDRFSGSGYGTDFTLTISSLQPEDFATYFCQQDYRSPWTFGGGTKVEIKGGGGSGGGGSGGGGSGGGSQVQLQESGPALVKPTQTLTLTCTVSGFSLSDYGVNWIRQPPGKALEWLGIIWGDGRTDYNSALKSRLSISKDNSKNQVVLRMTNVDPVDTATYFCARAPGNRAMEYWGQGTVVTVSSASTSGGGGSGGGGSGGGGSGGGGSGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSTSGSGKPGSGEGSTKGQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGSHHHHHH

SEQ ID NO: 104 - protein, 518 aa, synthetic construct
  REGION 1..518: note = Synthetic: IL-12 Cytokine Domain
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS